(19) United States
(12) Patent Application Publication
(10) Pub. No.: US 2013/0158023 A1
(43) Pub. Date: Jun. 20, 2013

(54) IDENTIFICATION OF GENE EXPRESSION AS A PREDICTIVE BIOMARKER FOR LKB1 STATUS

(71) Applicants: Yuhong L. Ning, San Diego, CA (US); Weiming Xu, San Diego, CA (US); Rajesh Chopra, Summit, NJ (US); Peter Worland, La Jolla, CA (US); Shuichan Xu, San Diego, CA (US)

(72) Inventors: Yuhong L. Ning, San Diego, CA (US); Weiming Xu, San Diego, CA (US); Rajesh Chopra, Summit, NJ (US); Peter Worland, La Jolla, CA (US); Shuichan Xu, San Diego, CA (US)

(73) Assignee: SIGNAL PHARMACEUTICALS, LLC, San Diego, CA (US)

(21) Appl. No.: 13/701,224

(22) PCT Filed: Aug. 2, 2012
(86) PCT No.: PCT/US2012/049281

§ 371 Date: Mar. 5, 2013

Related U.S. Application Data
(60) Provisional application No. 61/514,798, filed on Aug. 3, 2011.

(51) Int. Cl. A61K 31/5377; A61K 31/437; A61K 31/498; C12Q 1/68; A61K 31/53

(52) U.S. Cl. CPC A61K 31/5377; C12Q 1/6886; A61K 31/53; A61K 31/498; A61K 31/437
USPC 514/232.5; 435/6.12; 506/9; 514/234.2; 514/243; 514/249; 514/293

(57) ABSTRACT

Provided herein are methods for predicting the LKB1 status of a patient or a biological sample, comprising the measurement of particular gene expression levels relative to a set of reference levels that represent the gene expression level of a biological wild-type sample without LKB1 gene or protein loss or mutation and the gene expression level of a reference sample with LKB1 gene or protein loss or mutation. Further provided herein are methods for treating and/or preventing a cancer or a tumor syndrome in a patient, comprising administering an effective amount of a TOR kinase inhibitor to a patient having cancer or a tumor syndrome, characterized by particular gene expression levels.

This application is a U.S. national stage application of International Patent Application No. PCT/US2012/049281, filed Aug. 2, 2012, which claims the benefit of U.S. Provisional Application No. 61/514,798, filed Aug. 3, 2011, the entire contents of each of which are incorporated herein by reference.

### 1. FIELD

Provided herein are methods for predicting the LKB1 status of a patient or a biological sample, comprising the measurement of particular gene expression levels relative to a set of reference levels that represent the gene expression level of a biological wild-type sample without LKB1 gene or protein loss or mutation and the gene expression level of a reference sample with LKB1 gene or protein loss or mutation. Further provided herein are methods for treating and/or preventing a cancer or a tumor syndrome in a patient, comprising administering an effective amount of a TOR kinase inhibitor to a patient having cancer or a tumor syndrome, characterized by particular gene expression levels.

### 2. BACKGROUND

The connection between abnormal protein phosphorylation and the cause or consequence of diseases has been known for over 20 years. Accordingly, protein kinases have become a very important group of drug targets. See Cohen, Nat. Rev. Drug Disc., 1:309-315 (2002), Grimmiger et al. Nat. Rev. Drug Disc. 9(12):956-970 (2010). Various protein kinase inhibitors have been used clinically in the treatment of a wide variety of diseases, such as cancer and chronic inflammatory diseases, including diabetes and stroke. See Cohen, Eur. J. Biochem., 268:5001-5010 (2001), Protein Kinase Inhibitors for the Treatment of Disease: The Promise and the Problems, Handbook of Experimental Pharmacology, Springer Berlin Heidelberg, 167 (2005).

The protein kinases belong to a large and diverse family of enzymes that catalyze protein phosphorylation and play a critical role in cellular signaling. Protein kinases may exert positive or negative regulatory effects, depending upon their target protein. Protein kinases are involved in specific signaling pathways which regulate cell functions such as, but not limited to, metabolism, cell cycle progression, cell adhesion, vascular function, apoptosis, and angiogenesis. Malfunctions of cellular signaling have been associated with many diseases, the most characterized of which include cancer and diabetes. The regulation of signal transduction by cytokines and the association of signal molecules with protooncogenes and tumor suppressor genes have been well documented. Similarly, the connection between diabetes and related conditions, and deregulated levels of protein kinases, has been demonstrated. See e.g., Sridhar et al. Pharm. Res. 17(11):1345-1353 (2000). Viral infections and the conditions related thereto have also been associated with the regulation of protein kinases. Park et al. Cell 101(7): 777-787 (2000).

Protein kinases can be divided into broad groups based upon the identity of the amino acid(s) that they target (serine/threonine, tyrosine, lysine, and histidine). For example, tyrosine kinases include receptor tyrosine kinases (RTKs), such as growth factors and non-receptor tyrosine kinases, such as the src kinase family. There are also dual-specific protein kinases that target both tyrosine and serine/threonine, such as cyclin dependent kinases (CDKs) and mitogen-activated protein kinases (MAPKs).

Because protein kinases regulate nearly every cellular process, including metabolism, cell proliferation, cell differentiation, and cell survival, they are attractive targets for therapeutic intervention for various disease states. For example, cell-cycle control and angiogenesis, in which protein kinases play a pivotal role are cellular processes associated with numerous disease conditions such as, but not limited to, cancer, inflammatory diseases, abnormal angiogenesis and diseases related thereto, atherosclerosis, macular degeneration, diabetes, obesity, and pain.

Protein kinases have become attractive targets for the treatment of cancers. Fabbro et al. Pharm. Ther. 93:79-98 (2002). It has been proposed that the involvement of protein kinases in the development of human malignancies may occur by: (1) genomic rearrangements (e.g., BCR-ABL in chronic myelogenous leukemia), (2) mutations leading to constitutively active kinase activity, such as acute myelogenous leukemia and gastrointestinal tumors, (3) deregulation of kinase activity by activation of oncogenes or loss of tumor suppressor functions, such as in cancers with oncogenic RAS, (4) deregulation of kinase activity by over-expression, as in the case of EGFR and (5) ectopic expression of growth factors that can contribute to the development and maintenance of the neoplastic phenotype. Fabbro et al., Pharm. Ther. 93:79-98 (2002).

The elucidation of the intricacy of protein kinase pathways and the complexity of the relationship and interaction among and between the various protein kinases and kinase pathways highlights the importance of developing pharmaceutical agents capable of acting as protein kinase modulators, regulators or inhibitors that have beneficial activity on multiple kinases or multiple kinase pathways. Accordingly, there remains a need for new kinase modulators.

The protein named mTOR (mammalian target of rapamycin), also called FRAP, RAFTI or RAPT1), is a 2549-amino acid Ser/Thr protein kinase, that has been shown to be one of the most critical proteins in the mTOR/PI3K/Akt pathway that regulates cell growth and proliferation. Georgakis and Younes Expert Rev. Anticancer Ther. 6(1):131-140 (2006). mTOR exists within two complexes, mTORC1 and mTORC2. While mTORC1 is sensitive to rapamycin analogs (such as temsirolimus or everolimus), mTORC2 is largely rapamycin-insensitive. Notably, rapamycin is not a TOR kinase inhibitor. Several mTOR inhibitors have been or are being evaluated in clinical trials for the treatment of cancer. Temsirolimus was approved for use in renal cell carcinoma in 2007 and everolimus was approved in 2009 for renal cell carcinoma patients that have progressed on vascular endothelial growth factor receptor inhibitors. In addition, sirolimus was approved in 1999 for the prophylaxis of renal transplant rejection. The interesting but limited clinical success of these mTORC1 inhibitory compounds demonstrates the usefulness of mTOR inhibitors in the treatment of cancer and transplant rejection, and the increased potential for compounds with both mTORC1 and mTORC2 inhibitory activity.

Somatic mutations affect key pathways in lung cancer. Accordingly, identification of specific mutations associated with lung cancer may lead to improved therapeutic protocols. Recent studies have uncovered a large number of somatic mutations of the LKB1 gene that are present in lung, cervical, breast, intestinal, testicular, pancreatic and skin cancer (Distribution of somatic mutations in STK11, Catalogue of Somatic Mutations in Cancer, Wellcome Trust Genome Campus, Hinxton, Cambridge).

Citation or identification of any reference in Section 2 of this application is not to be construed as an admission that the reference is prior art to the present application.

### 3. SUMMARY

Provided herein are methods for predicting the LKB1 status of a patient or a biological sample, comprising the measurement of a predictive gene expression level. Without being limited by theory, it is believed that certain gene expression levels are characteristic of LKB1 gene and/or protein mutation and/or loss.

Further provided herein are methods for treating or preventing a cancer, for example non-small cell lung carcinoma or cervical cancer, or treating a tumor syndrome, for example Peutz-Jeghers Syndrome, comprising administering an effective amount of a TOR kinase inhibitor to a patient having a cancer or a tumor syndrome characterized by a particular gene expression level, relative to that of wild type.

Further provided herein are methods for treating or preventing a cancer, for example non-small cell lung carcinoma or cervical cancer, comprising screening a patient's cancer for the presence of a particular gene expression level relative to that of wild type and administering an effective amount of a TOR kinase inhibitor to the patient having a cancer characterized by a particular gene expression level.

Further provided herein are methods for predicting LKB1 gene and/or protein loss and/or mutation in a patient's (“test patient”) cancer, for example non-small cell lung carcinoma or cervical cancer, comprising: a) obtaining a biological test sample from the patient's cancer; b) obtaining the gene expression level(s) of one or more genes selected from Table 1 in said biological sample; c) comparing said gene expression level(s) to a set of reference levels that represent the gene expression level(s) of a biological wild-type sample without LKB1 gene and/or protein loss and/or mutation, and the gene expression level(s) of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of an LKB1 gene or protein loss or mutation in the patient's cancer.

Further provided herein are methods for treating non-small cell lung carcinoma, cervical cancer or Peutz-Jeghers Syndrome, comprising administering an effective amount of a TOR kinase inhibitor to a patient having non-small cell lung carcinoma, cervical cancer or Peutz-Jeghers Syndrome, wherein the gene expression level(s) of a biological test sample from said patient is characterized by higher similarity to the gene expression level(s) of a reference sample with LKB1 gene and/or protein loss and/or mutation than the gene expression level(s) of a wild type sample without LKB1 gene and/or r protein loss and/or mutation, and wherein the genes are selected from Table 1.

Further provided are methods for treating non-small cell lung carcinoma or cervical cancer, comprising screening a patient's carcinoma or cancer for the presence of LKB1 gene and/or protein loss and/or mutation, relative to wild type, and administering an effective amount of a TOR kinase inhibitor to the patient having non-small cell lung carcinoma or cervical cancer characterized by a gene expression level characterized by higher similarity to the gene expression level(s) of a reference sample with LKB1 gene and/or protein loss and/or mutation than the gene expression level(s) of a wild type sample without LKB1 gene and/or protein loss and/or mutation, and wherein the genes are selected from Table 1.

Further provided herein are methods for predicting response to treatment with a TOR kinase inhibitor in a patient having cancer, for example non-small cell lung carcinoma or cervical cancer, the method comprising: a) obtaining a biological test sample from the patient's cancer; b) obtaining the gene expression level(s) of one or more genes selected from Table 1 in said biological test sample; c) comparing said gene expression level(s) to a set of reference levels that represent the gene expression level(s) of a biological wild-type sample without LKB1 gene and/or protein loss and/or mutation and the gene expression level(s) of a reference sample with LKB1 gene and/or protein loss and/or mutation; wherein the gene expression level(s) of the biological test sample characterized by higher similarity to the gene expression level(s) of a reference sample with LKB1 gene and/or protein loss and/or mutation, indicates an increased likelihood of response to TOR kinase inhibitor treatment of said patient's cancer.

Further provided herein are methods for predicting therapeutic efficacy of TOR kinase inhibitor treatment of a patient having cancer, for example non-small cell lung carcinoma or cervical cancer, with a TOR kinase inhibitor, the method comprising: a) obtaining a biological test sample from the patient's cancer; b) obtaining the gene expression level(s) of one or more genes selected from Table 1 in said biological test sample; c) comparing said gene expression level(s) to a set of reference levels that represent the gene expression level(s) of a biological wild-type sample without LKB1 gene and/or protein loss and/or mutation and the gene expression level(s) of a reference sample with LKB1 gene and/or protein loss and/or mutation; wherein the gene expression level(s) of the biological test sample characterized by higher similarity to the gene expression level(s) of a reference sample with LKB1 gene and/or protein loss and/or mutation, indicates an increased likelihood of therapeutic efficacy of said TOR kinase inhibitor treatment for said patient.

Further provided herein are methods of screening a patient having cancer, for example non-small cell lung carcinoma or cervical cancer, for LKB1 gene and/or protein loss and/or mutation, the method comprising: a) obtaining a biological test sample from the patient's cancer; b) obtaining the gene expression level(s) of one or more genes selected from Table 1 in said biological test sample; c) comparing said gene expression level(s) to a set of reference levels that represent the gene expression level(s) of a biological wild-type sample without LKB1 gene and/or protein loss and/or mutation and the gene expression level(s) of a reference sample with LKB1 gene and/or protein loss and/or mutation; wherein the gene expression level(s) of the biological test sample characterized by higher similarity to the gene expression level(s) of a reference sample with LKB1 gene and/or protein loss and/or mutation, indicates an increased likelihood of LKB1 gene and/or protein loss and/or mutation.

Further provided herein are methods for treating a tumor syndrome, for example Peutz-Jeghers Syndrome, comprising comparing a patient's gene expression level(s) to wild type, and administering an effective amount of a TOR kinase inhibitor to the patient having a tumor syndrome characterized by a gene expression level(s) characterized by higher similarity to the gene expression level(s) of a reference sample with LKB1 gene and/or protein loss and/or mutation than the gene expression level(s) of a wild type sample without LKB1 gene and/or protein loss and/or mutation, and wherein the genes are selected from Table 1.

Further provided are methods for treating a tumor syndrome, for example Peutz-Jeghers Syndrome, comprising screening a patient for the presence of LKB1 gene and/or protein loss and/or mutation, relative to wild type, and administering an effective amount of a TOR kinase inhibitor to the patient having a tumor syndrome characterized by a gene expression level(s) characterized by higher similarity to the gene expression level(s) of a reference sample with LKB1 gene and/or protein loss and/or mutation than the gene expression level(s) of a wild type sample without LKB1 gene and/or protein loss and/or mutation, and wherein the genes are selected from Table 1.

Further provided herein are methods for predicting LKB1 gene and/or protein loss and/or mutation in a patient having a tumor syndrome, for example, Peutz-Jeghers Syndrome, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level(s) of a biological wild-type sample without LKB1 gene and/or protein loss and/or mutation and the gene expression level(s) of a reference sample with LKB1 gene and/or protein loss and/or mutation; wherein the gene expression level(s) of the biological test sample characterized by higher similarity to the gene expression level(s) of a reference sample with LKB1 gene and/or protein loss and/or mutation, indicates an increased likelihood of an LKB1 gene and/or protein loss and/or mutation in the patient.

Further provided herein are methods for predicting response to TOR kinase inhibitor therapy in a patient having a tumor syndrome, for example, Peutz-Jeghers Syndrome, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level(s) of a biological wild-type sample without LKB1 gene and/or protein loss and/or mutation and the gene expression level(s) of a reference sample with LKB1 gene and/or protein loss and/or mutation; wherein the gene expression level(s) of the biological test sample characterized by higher similarity to the gene expression level(s) of a reference sample with LKB1 gene and/or protein loss and/or mutation, indicates an increased likelihood of response to TOR kinase inhibitor treatment of said patient's tumor syndrome.

Further provided herein are methods for predicting therapeutic efficacy of treatment of a patient having a tumor syndrome, for example, Peutz-Jeghers Syndrome, with a TOR kinase inhibitor, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level(s) of a biological wild-type sample without LKB1 gene and/or protein loss and/or mutation and the gene expression level(s) of a reference sample with LKB1 gene and/or protein loss and/or mutation; wherein the gene expression level(s) of the biological test sample characterized by higher similarity to the gene expression level(s) of a reference sample with LKB1 gene and/or protein loss and/or mutation, indicates an increased likelihood of therapeutic efficacy of said TOR kinase inhibitor treatment for said patient.

Further provided herein are methods of screening a patient having a tumor syndrome, for example Peutz-Jeghers Syndrome, for LKB1 gene and/or protein loss and/or mutation, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level(s) of a biological wild-type sample without LKB1 gene and/or protein loss and/or mutation and the gene expression level(s) of a reference sample with LKB1 gene and/or protein loss and/or mutation; wherein the gene expression level(s) of the biological test sample characterized by higher similarity to the gene expression level(s) of a reference sample with LKB1 gene and/or protein loss and/or mutation, indicates an increased likelihood for LKB1 gene and/or protein loss and/or mutation.

In certain embodiments provided herein, the gene expression level of the biological test sample is obtained using gene mRNA measurement. In certain of the methods and embodiments provided herein, the gene expression level of the biological test sample is obtained using RT-PCR or Affymetrix HGU133plus2. In some embodiments, comparison of gene expression levels is performed using Prediction Analysis of Microarrays for R (“PAMR”) (http://cran.r-project.org/web/packages/pamr/pamr.pdf).

Further provided herein are kits comprising one or more containers filled with a TOR kinase inhibitor or a pharmaceutical composition thereof, reagents for measuring gene expression levels of a patient's cancer or of a patient having a tumor syndrome and instructions for measuring gene expression levels of a patient's cancer or of a patient having a tumor syndrome.

In some embodiments, the TOR kinase inhibitor is a compound as described herein.

The present embodiments can be understood more fully by reference to the detailed description and examples, which are intended to exemplify non-limiting embodiments.

### 4. BRIEF DESCRIPTION OF THE DRAWINGS

FIG. 1 provides a heatmap of the gene expression level of certain LKB1 positive (wild type) and negative (LKB1 gene and/or protein loss and/or mutation) non-small cell lung cancer cell types obtained using prediction analysis of microarrays (PAM) extraction.

FIG. 2 provides a list of enriched GeneOntology groups.

FIG. 3 lists the LKB1 mutation status of non small cell lung cancer (NSCLC) cell lines, based on reported DNA sequences, the reported mutation, the presence (positive) or absence (negative) of intact LKB1 protein (as determined by Western immunoblotting).

FIG. 4. FIG. 4 provides a list of enriched pathway groups.

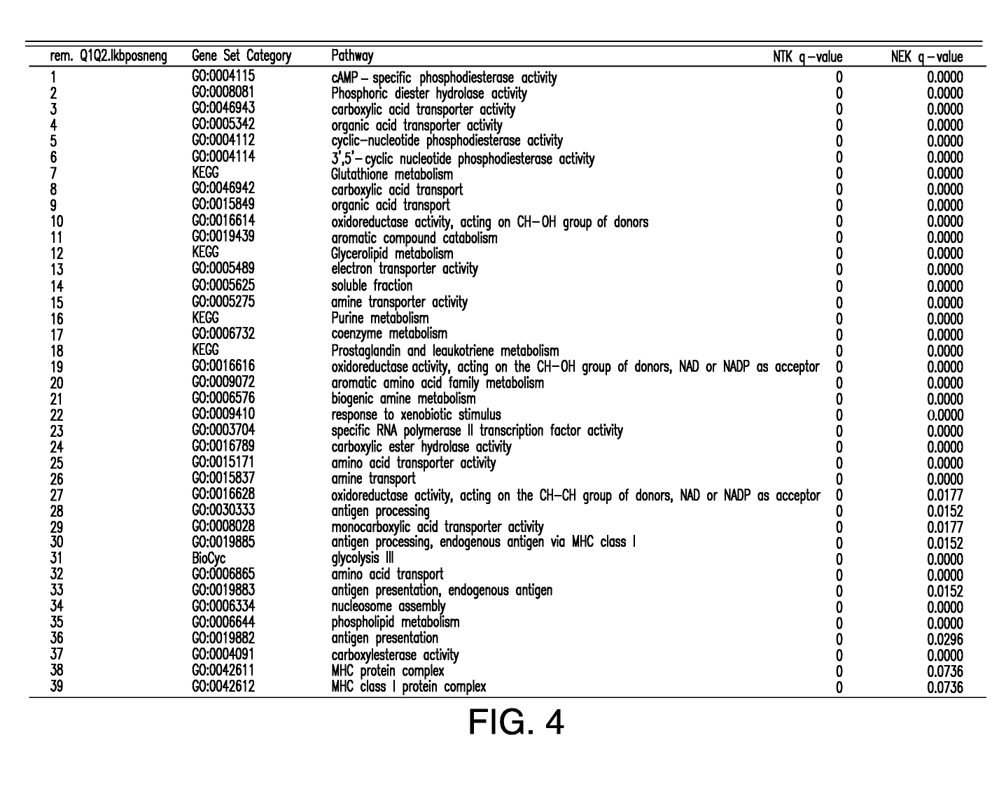

### 5. DETAILED DESCRIPTION

### 5.1 Definitions

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. Unless otherwise indicated, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxylamine; alkoxyamine; aralkoxyamine; N-oxide; hydrazine; hydrazide; hydrazone; azide; isocyanate; isothiocyanate; cyanate; thiocyanate; oxygen (═O); B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl and the like. The double bond of an alkenyl group can be unconjugated or conjugated to another unsaturated group. An alkenyl group can be unsubstituted or substituted.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclylalkyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono- substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R^{#} are independently as defined above.

An “alkylsulfonylamino” group is a radical of the formula: —NHSO₂(R^{#}) or —N(alkyl)SO₂(R^{#}), wherein each alkyl and R^{#} are defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxylamine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the TOR kinase inhibitors include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “clathrate” means a TOR kinase inhibitor, or a salt thereof, in the form of a crystal lattice that contains spaces (e.g., channels) that have a guest molecule (e.g., a solvent or water) trapped within or a crystal lattice wherein a TOR kinase inhibitor is a guest molecule.

As used herein and unless otherwise indicated, the term “solvate” means a TOR kinase inhibitor, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a TOR kinase inhibitor, or a salt thereof, that further includes a stoichiometric or non-stoichiometric amount of water bound by non-covalent intermolecular forces.

As used herein and unless otherwise indicated, the term “prodrug” means a TOR kinase inhibitor derivative that can hydrolyze, oxidize, or otherwise react under biological conditions (in vitro or in vivo) to provide an active compound, particularly a TOR kinase inhibitor. Examples of prodrugs include, but are not limited to, derivatives and metabolites of a TOR kinase inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a TOR kinase inhibitor that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The TOR kinase inhibitors can have chiral centers and can occur as racemates, individual enantiomers or diastereomers, and mixtures thereof. All such isomeric forms are included within the embodiments disclosed herein, including mixtures thereof. The use of stereomerically pure forms of such TOR kinase inhibitors, as well as the use of mixtures of those forms are encompassed by the embodiments disclosed herein. For example, mixtures comprising equal or unequal amountsv of the enantiomers of a particular TOR kinase inhibitor may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the TOR kinase inhibitors can include E and Z isomers, or a mixture thereof, and cis and trans isomers or a mixture thereof. In certain embodiments, the TOR kinase inhibitors are isolated as either the cis or trans isomer. In other embodiments, the TOR kinase inhibitors are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

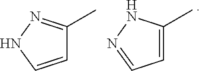

As readily understood by one skilled in the art, a wide variety of functional groups and other structures may exhibit tautomerism and all tautomers of the TOR kinase inhibitors are within the scope of the present invention.

It should also be noted the TOR kinase inhibitors can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14) or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer and inflammation therapeutic agents, research reagents, e.g., binding assay reagents, and diagnostic agents, e.g., in vivo imaging agents. All isotopic variations of the TOR kinase inhibitors as described herein, whether radioactive or not, are intended to be encompassed within the scope of the embodiments provided herein. In some embodiments, there are provided isotopologues of the TOR kinase inhibitors, for example, the isotopologues are deuterium, carbon-13, or nitrogen-15 enriched TOR kinase inhibitors.

“Treating” as used herein, means an alleviation, in whole or in part, of symptoms associated with a disorder or disease (e.g., cancer or a tumor syndrome), or slowing, or halting of further progression or worsening of those symptoms.

“Preventing” as used herein, means the prevention of the onset, recurrence or spread, in whole or in part, of the disease or disorder (e.g., cancer), or a symptom thereof.

The term “effective amount” in connection with an TOR kinase inhibitor means an amount capable of alleviating, in whole or in part, symptoms associated with cancer, for example non-small cell lung carcinoma or cervical cancer, or a tumor syndrome, for example Peutz-Jeghers Syndrome, or slowing or halting further progression or worsening of those symptoms, or preventing or providing prophylaxis for cancer, for example non-small cell lung carcinoma or cervical cancer, or a tumor syndrome, for example Peutz-Jeghers Syndrome in a subject at risk for cancer, for example non-small cell lung carcinoma or cervical cancer, or a tumor syndrome, for example Peutz-Jeghers Syndrome. The effective amount of the TOR kinase inhibitor, for example in a pharmaceutical composition, may be at a level that will exercise the desired effect; for example, about 0.005 mg/kg of a subject's body weight to about 100 mg/kg of a patient's body weight in unit dosage for both oral and parenteral administration. As will be apparent to those skilled in the art, it is to be expected that the effective amount of a TOR kinase inhibitor disclosed herein may vary depending on the severity of the indication being treated.

As used herein “wild type” refers to the typical or most common form of a characteristic (for example, gene sequence or presence, or protein sequence, presence, level or activity), as it occurs in nature, and the reference against which all others are compared. As will be understood by one skilled in the art, when used herein, wild type refers to the typical gene expression levels as they most commonly occur in nature. Similarly, a “control patient”, as used herein, is a patient who exhibits the wild type gene expression levels. In certain embodiments, the gene expression level is comprised of the gene expression level of one or more of the genes set forth in Table 1.

As used herein “LKB1 gene or protein mutation” refers to, for example, a LKB1 gene mutation resulting in a decrease in LKB1 mRNA expression, a decrease in LKB1 protein production or a non-functional LKB1 protein, as compared to wild type. As used herein “LKB1 gene or protein loss” refers to a reduced level of LKB1 protein or the absence of LKB1 protein, as compared to wild type levels. The phrase “LKB1 gene and/or protein loss and/or mutation” includes each of the following, alone or in combination with one or more of the others: (1) LKB1 gene loss; (2) LKB1 gene mutation; (3) LKB1 protein loss; and (4) LKB1 protein mutation.

As used herein “reduced level” or “loss” means a reduction in level relative to levels observed in wild type. In one embodiment the reduction is 10%-50% or 50%-100%. In some embodiments, the reduction is 20%, 30%, 40%, 50%, 60%, 70%, 80%. 90% or 100% (complete loss) relative to wild type.

As used herein in connection with the comparison of gene expression level(s) of a biological test sample with wild-type samples and/or reference samples, “similarity” is determined using the Nearest Shrunken Centroid Method (see Tibsharani et al., PNAS 99: 6567-6572 (2002). The Nearest Shrunken Centroid Method computes a standardized centroid for each class of samples, for example, wild type samples and reference samples. This centroid is the average gene expression level for each gene in each class divided by the within-class standard deviation for that gene. Nearest centroid classification then takes the gene expression profile of a new sample (e.g., biological test sample) and compares it to each of these class centroids. The class (e.g., the reference sample or the wild-type sample) whose centroid that the gene expression profile of the new sample is closest to, in squared distance, is the predicted class or the class the new sample has the higher similarity to. As used herein, “higher similarity” of the biological test sample means that the gene expression level(s) of the biological sample is determined to be more similar to either the reference levels that represent the gene expression level(s) of a biological wild-type sample without LKB1 gene and/or protein loss and/or mutation (LKB1 positive, or wild type) or the gene expression level(s) of a reference sample with LKB1 gene and/or protein loss and/or mutation (LKB1 negative).

The terms “patient” and “subject” as used herein include an animal, including, but not limited to, an animal such as a cow, monkey, horse, sheep, pig, chicken, turkey, quail, cat, dog, mouse, rat, rabbit or guinea pig, in one embodiment a mammal, in another embodiment a human.

In one embodiment, a “patient” or “subject” is a human whose cancer DNA comprises a LKB1 gene mutation, relative to that of a control patient or wild type. In another embodiment, a “patient” or “subject” is a human whose cancer DNA contains a LKB1 gene mutation, relative to that of a control patient or wild type. In another embodiment, a “patient” or “subject” is a human having a cancer, for example non-small cell lung carcinoma or cervical cancer, characterized by LKB1 gene and/or protein loss and/or mutation, relative to that of a control patient or wild type. In particular embodiments, the LKB1 gene and/or protein loss and/or mutation is identified by certain gene expression levels, measured using RT-PCR or the Affymetrix HGU133plus2 platform, and compared to wild type using the statistical package PAMR. In certain embodiments, the gene expression level is comprised of the gene expression levels of one or more of the genes set forth in Table 1.

In another embodiment, a “patient” or “subject” is a human whose DNA comprises a LKB1 gene mutation, relative to that of a control patient or wild type. In another embodiment, a “patient” or “subject” is a human whose DNA contains a LKB1 gene mutation, relative to that of a control patient or wild type. In another embodiment, a “patient” or “subject” is a human having LKB1 gene and/or protein loss and/or mutation, relative to that of a control patient or wild type. In another embodiment, a “patient” or “subject” is a human having LKB1 gene and/or protein loss and/or mutation, relative to that of a control patient or wild type, and also having a tumor syndrome, for example Peutz-Jeghers Syndrome. In particular embodiments, the LKB1 gene and/or protein loss and/or mutation is identified by certain gene expression levels measured using RT-PCR or the Affymetrix HGU133plus2 platform and compared to wild type using the statistical package PAMR. In certain embodiments, the gene expression level is comprised of the gene expression levels of one or more of the genes set forth in Table 1.

The term “expression” as used herein refers to the transcription from a gene to give an RNA nucleic acid molecule at least complementary in part to a region of one of the two nucleic acid strands of the gene. The term “expression” as used herein also refers to the translation from the RNA molecule to give a protein, a polypeptide or a portion thereof.

The expression of a gene that is “upregulated” is generally “increased” relative to wild type. The expression of a gene that is “downregulated” is generally “decreased” relative to wild type. In certain embodiments, a gene from a patient sample can be “upregulated,” i.e., gene expression can be increased, for example, by about 5%, 10%, 20%, 30%, 40%, 50%, 60%, 70%, 90%, 100%, 200%, 300%, 500%, 1,000%, 5,000% or more of a comparative control, such as wild type. In other embodiments, a gene from a patient sample can be “downregulated,” i.e., gene expression can be decreased, for example, by about 99%, 95%, 90%, 80%, 70%, 60%, 50%, 40%, 30%, 20%, 10%, 1% or less of a comparative control, such as wild type.

The term “likelihood” generally refers to an increase in the probability of an event. The term “likelihood” when used in reference to the effectiveness of a patient response generally contemplates an increased probability that a cancer or tumor syndrome, or symptom thereof, will be lessened or decreased.

The term “predict” generally means to determine or tell in advance. When used to “predict” the effectiveness of a cancer or tumor syndrome treatment, for example, the term “predict” can mean that the likelihood of the outcome of the treatment can be determined at the outset, before the treatment has begun, or before the treatment period has progressed substantially.

The terms “determining”, “measuring”, “evaluating”, “assessing” and “assaying” as used herein generally refer to any form of measurement, and include determining if an element is present or not. These terms include both quantitative and/or qualitative determinations.

In the context of cancer, for example non-small cell lung carcinoma or cervical cancer, or a tumor syndrome, for example Peutz-Jeghers Syndrome, inhibition may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention. In this context, the term “prevention” includes either preventing the onset of clinically evident cancer, carcinoma or tumor altogether or preventing the onset of a preclinically evident stage of cancer, carcinoma or tumor in individuals at risk. Also intended to be encompassed by this definition is the prevention of transformation into malignant cells or to arrest or reverse the progression of premalignant cells to malignant cells. This includes prophylactic treatment of those at risk of developing the cancer, carcinoma or tumor.

### 5.2 Gene Expression Profile

Table 1 sets forth the genes for which the gene expression compared to wild type, indicate a higher likelihood of LKB1 gene and/or protein loss and/or mutation.

| | | | Fold Change |
|---|---|---|---|
| | | | (LKB1 Pos/ |
| Probe | Symbol | Gene Name | LKB1 Neg) |
| 229839_at | SCARA5 | scavenger receptor class A, member 5 | −120.683 |
| | | (putative) | |
| 219612_s_at | FGG | fibrinogen gamma chain | −67.798 |
| 235849_at | SCARA5 | scavenger receptor class A, member 5 | −35.633 |
| | | (putative) | |
| 205649_s_at | FGA | fibrinogen alpha chain | −30.292 |
| 206549_at | INSL4 | insulin-like 4 (placenta) | −24.589 |
| 230830_at | OSTbeta | organic solute transporter beta | −24.265 |
| 205650_s_at | FGA | fibrinogen alpha chain | −22.98 |
| 231213_at | PDE1A | phosphodiesterase 1A, calmodulin- | −20.621 |
| | | dependent | |
| 217564_s_at | CPS1 | carbamoyl-phosphate synthetase 1, | −19.299 |
| | | mitochondrial | |
| 241535_at | NA | NA | −17.5 |
| 219764_at | FZD10 | frizzled homolog 10 (Drosophila) | −16.83 |
| 214303_x_at | MUCSAC | mucin 5AC, oligomeric mucus/gel-forming | −16.35 |
| 204920_at | CPS1 | carbamoyl-phosphate synthetase 1, | −15.906 |
| | | mitochondrial | |
| 244567_at | NA | NA | −15.493 |
| 205009_at | TFF1 | trefoil factor 1 | −15.383 |
| 206528_at | TRPC6 | transient receptor potential cation channel, | −14.276 |
| | | subfamily C, member 6 | |
| 205403_at | IL1R2 | interleukin 1 receptor, type II | −12.759 |
| 216238_s_at | FGB | fibrinogen beta chain | −12.071 |
| 229778_at | C12orf39 | chromosome 12 open reading frame 39 | −11.705 |
| 1569378_at | FLJ33297 | hypothetical gene supported by AK090616 | −10.97 |
| 228186_s_at | RSPO3 | R-spondin 3 homolog (Xenopus laevis) | −10.185 |
| 204988_at | FGB | fibrinogen beta chain | −10.144 |
| 211372_s_at | IL1R2 | interleukin 1 receptor, type II | −10.037 |
| 213432_at | MUC5B | mucin 5B, oligomeric mucus/gel-forming | −9.855 |
| 222712_s_at | MUC13 | mucin 13, cell surface associated | −9.374 |
| 214385_s_at | MUC5AC | mucin 5AC, oligomeric mucus/gel-forming | −9.346 |
| 206153_at | CYP4F11 | cytochrome P450, family 4, subfamily F, | −8.054 |
| | | polypeptide 11 | |
| 228969_at | AGR2 | anterior gradient homolog 2 (Xenopus | −8.022 |
| | | laevis) | |
| 207052_at | HAVCR1 | hepatitis A virus cellular receptor 1 | −7.623 |
| 205305_at | FGL1 | fibrinogen-like 1 | −7.594 |
| 232687_at | NA | NA | −7.471 |
| 212224_at | ALDH1A1 | aldehyde dehydrogenase 1 family, member A1 | −7.208 |
| 230251_at | C6orf176 | chromosome 6 open reading frame 176 | −7.171 |
| 212935_at | MCF2L | MCF.2 cell line derived transforming | −6.718 |
| | | sequence-like | |
| 226992_at | NOSTRIN | nitric oxide synthase trafficker | −6.706 |
| 215189_at | KRT86 | keratin 86 | −6.706 |
| 228507_at | NA | NA | −6.645 |
| 220479_at | LOC29034 | hypothetical LOC29034 | −6.621 |
| 232267_at | GPR133 | G protein-coupled receptor 133 | −6.25 |
| 205137_x_at | USH1C | Usher syndrome 1C (autosomal recessive, | −6.104 |
| | | severe) | |
| 206515_at | CYP4F3 | cytochrome P450, family 4, subfamily F, | −5.965 |
| | | polypeptide 3 | |
| 205221_at | HGD | homogentisate 1,2-dioxygenase | −5.952 |
| | | (homogentisate oxidase) | |
| 233413_at | NA | NA | −5.911 |
| 229655_at | FAM19A5 | family with sequence similarity 19 | −5.616 |
| | | (chemokine (C-C motif)-like), member A5 | |
| 239650_at | NCKAP5 | NCK-associated protein 5 | −5.516 |
| 236300_at | NA | NA | −5.51 |
| 209616_s_at | CES1 | carboxylesterase 1 (monocyte/macrophage | −5.483 |
| | | serine esterase 1) | |
| 226248_s_at | KIAA1324 | KIAA1324 | −5.47 |
| 218541_s_at | C8orf4 | chromosome 8 open reading frame 4 | −5.395 |
| 242426_at | NRG4 | neuregulin 4 | −5.273 |
| 209959_at | NR4A3 | nuclear receptor subfamily 4, group A, | −5.267 |
| | | member 3 | |
| 232608_x_at | CARD14 | caspase recruitment domain family, member 14 | −5.249 |
| 228057_at | DDIT4L | DNA-damage-inducible transcript 4-like | −5.175 |
| 206155_at | ABCC2 | ATP-binding cassette, sub-family C | −5.122 |
| | | (CFTR/MRP), member 2 | |
| 201884_at | CEACAM5 | carcinoembryonic antigen-related cell | −5.04 |
| | | adhesion molecule 5 | |
| 228962_at | PDE4D | phosphodiesterase 4D, cAMP-specific | −4.976 |
| | | (phosphodiesterase E3 dunce homolog, | |
| | | Drosophila) | |
| 237471_at | tcag7.1307 | hypothetical LOC154822 | −4.847 |
| 206389_s_at | PDE3A | phosphodiesterase 3A, cGMP-inhibited | −4.798 |
| 233076_at | JAKMIP3 | Janus kinase and microtubule interacting | −4.794 |
| | | protein 3 | |
| 206645_s_at | NR0B1 | nuclear receptor subfamily 0, group B, | −4.779 |
| | | member 1 | |
| 210663_s_at | KYNU | kynureninase (L-kynurenine hydrolase) | −4.746 |
| 210662_at | KYNU | kynureninase (L-kynurenine hydrolase) | −4.689 |
| 202023_at | EFNA1 | ephrin-A1 | −4.639 |
| 238755_at | NA | NA | −4.622 |
| 234563_at | NA | NA | −4.521 |
| 238029_s_at | SLC16A14 | solute carrier family 16, member 14 | −4.489 |
| | | (monocarboxylic acid transporter 14) | |
| 1556331_a_at | NA | NA | −4.391 |
| 205234_at | SLC16A4 | solute carrier family 16, member 4 | −4.378 |
| | | (monocarboxylic acid transporter 5) | |
| 236741_at | WDR72 | WD repeat domain 72 | −4.351 |
| 214308_s_at | HGD | homogentisate 1,2-dioxygenase | −4.346 |
| | | (homogentisate oxidase) | |
| 219049_at | CSGALNACT1 | chondroitin sulfate N- | −4.339 |
| | | acetylgalactosaminyltransferase 1 | |
| 204385_at | KYNU | kynureninase (L-kynurenine hydrolase) | −4.325 |
| 220393_at | LGSN | lengsin, lens protein with glutamine | −4.273 |
| | | synthetase domain | |
| 206644_at | NR0B1 | nuclear receptor subfamily 0, group B, | −4.259 |
| | | member 1 | |
| 204491_at | PDE4D | phosphodiesterase 4D, cAMP-specific | −4.236 |
| | | (phosphodiesterase E3 dunce homolog, | |
| | | Drosophila) | |
| 219508_at | GCNT3 | glucosaminyl (N-acetyl) transferase 3, | −4.17 |
| | | mucin type | |
| 211184_s_at | USH1C | Usher syndrome 1C (autosomal recessive, | −4.135 |
| | | severe) | |
| 204014_at | DUSP4 | dual specificity phosphatase 4 | −4.114 |
| 229280_s_at | FLJ22536 | hypothetical locus LOC401237 | −4.077 |
| 219300_s_at | CNTNAP2 | contactin associated protein-like 2 | −3.97 |
| 203963_at | CA12 | carbonic anhydrase XII | −3.96 |
| 204351_at | Sl00P | S100 calcium binding protein P | −3.95 |
| 203238_s_at | NOTCH3 | Notch homolog 3 (Drosophila) | −3.945 |
| 214307_at | HGD | homogentisate 1,2-dioxygenase | −3.942 |
| | | (homogentisate oxidase) | |
| 206561_s_at | AKR1B10 | aldo-keto reductase family 1, member B10 | −3.937 |
| | | (aldose reductase) | |
| 226034_at | NA | NA | −3.935 |
| 205477_s_at | AMBP | alpha-1-microglobulin/bikunin precursor | −3.897 |
| 1555854_at | NA | NA | −3.893 |
| 217626_at | NA | NA | −3.871 |
| 205501_at | PDE10A | phosphodiesterase 10A | −3.741 |
| 217388 _s_at | KYNU | kynureninase (L-kynurenine hydrolase) | −3.734 |
| 220540_at | KCNK15 | potassium channel, subfamily K, member 15 | −3.669 |
| 209173_at | AGR2 | anterior gradient homolog 2 (Xenopus | −3.657 |
| | | laevis) | |
| 211840_s_at | PDE4D | phosphodiesterase 4D, cAMP-specific | −3.646 |
| | | (phosphodiesterase E3 dunce homolog, | |
| | | Drosophila) | |
| 219429_at | FA2H | fatty acid 2-hydroxylase | −3.627 |
| 227614_at | HKDC1 | hexokinase domain containing 1 | −3.615 |
| 206643_at | HAL | histidine ammonia-lyase | −3.593 |
| 204105_s_at | NRCAM | neuronal cell adhesion molecule | −3.567 |
| 205460_at | NPAS2 | neuronal PAS domain protein 2 | −3.548 |
| 39248_at | AQP3 | aquaporin 3 (Gill blood group) | −3.544 |
| 216248_s_at | NR4A2 | nuclear receptor subfamily 4, group A, | −3.519 |
| | | member 2 | |
| 212906_at | GRAMD1B | GRAM domain containing 1B | −3.514 |
| 227202_at | CNTN1 | contactin 1 | −3.498 |
| 221577_x_at | GDF15 | growth differentiation factor 15 | −3.484 |
| 240173_at | NA | NA | −3.466 |
| 242871_at | PAQR5 | progestin and adipoQ receptor family | −3.432 |
| | | member V | |
| 242626_at | SAMD5 | sterile alpha motif domain containing 5 | −3.379 |
| 222784_at | SMOC1 | SPARC related modular calcium binding 1 | −3.34 |
| 1562102_at | AKR1C1 | aldo-keto reductase family 1, member C1 | −3.314 |
| | | (dihydrodiol dehydrogenase 1; 20-alpha (3- | |
| | | alpha)-hydroxysteroid dehydrogenase) | |
| 204622_x_at | NR4A2 | nuclear receptor subfamily 4, group A, | −3.313 |
| | | member 2 | |
| 202388_at | RGS2 | regulator of G-protein signaling 2, 24 kDa | −3.312 |
| 226192_at | NA | NA | −3.283 |
| 202889_x_at | MAP7 | microtubule-associated protein 7 | −3.26 |
| 227209_at | CNTN1 | contactin 1 | −3.244 |
| 204621_s_at | NR4A2 | nuclear receptor subfamily 4, group A, | −3.233 |
| | | member 2 | |
| 227174_at | WDR72 | WD repeat domain 72 | −3.199 |
| 1556698_a_at | GPRIN3 | GPRIN family member 3 | −3.199 |
| 233177_s_at | PNKD | paroxysmal nonkinesigenic dyskinesia | −3.193 |
| 210837_s_at | PDE4D | phosphodiesterase 4D, cAMP-specific | −3.117 |
| | | (phosphodiesterase E3 dunce homolog, | |
| | | Drosophila) | |
| 243438_at | PDE7B | phosphodiesterase 7B | −3.081 |
| 205698_s_at | MAP2K6 | mitogen-activated protein kinase kinase 6 | −3.077 |
| 203708_at | PDE4B | phosphodiesterase 4B, cAMP-specific | −3.069 |
| | | (phosphodiesterase E4 dunce homolog, | |
| | | Drosophila) | |
| 241726_at | NA | NA | −3.047 |
| 202986_at | ARNT2 | aryl-hydrocarbon receptor nuclear | −3.045 |
| | | translocator 2 | |
| 222783_s_at | SMOC1 | SPARC related modular calcium binding 1 | −3.026 |
| 1554717_a_at | PDE4D | phosphodiesterase 4D, cAMP-specific | −2.97 |
| | | (phosphodiesterase E3 dunce homolog, | |
| | | Drosophila) | |
| 244387_at | NA | NA | −2.964 |
| 218631_at | AVPI1 | arginine vasopressin-induced 1 | −2.938 |
| 228653_at | SAMD5 | sterile alpha motif domain containing 5 | −2.92 |
| 1569433_at | SAMD5 | sterile alpha motif domain containing 5 | −2.903 |
| 221667_s_at | HSPB8 | heat shock 22 kDa protein 8 | −2.886 |
| 214240_at | GAL | galanin prepropeptide | −2.886 |
| 237029_at | HGD | homogentisate 1,2-dioxygenase | −2.868 |
| | | (homogentisate oxidase) | |
| 225516_at | SLC7A2 | solute carrier family 7 (cationic amino acid | −2.845 |
| | | transporter, y+ system), member 2 | |
| 230563_at | RASGEF1A | RasGEF domain family, member 1A | −2.843 |
| 222073_at | COL4A3 | collagen, type IV, alpha 3 (Goodpasture | −2.83 |
| | | antigen) | |
| 236610_at | NA | NA | −2.819 |
| 205311_at | DDC | dopa decarboxylase (aromatic L-amino acid | −2.809 |
| | | decarboxylase) | |
| 206017_at | KIAA0319 | KIAA0319 | −2.801 |
| 221067_s_at | C12orf39 | chromosome 12 open reading frame 39 | −2.765 |
| 238498_at | NA | NA | −2.762 |
| 204015_s_at | DUSP4 | dual specificity phosphatase 4 | −2.757 |
| 215471_s_at | MAP7 | microtubule-associated protein 7 | −2.736 |
| 237031_at | NA | NA | −2.719 |
| 203747_at | AQP3 | aquaporin 3 (Gill blood group) | −2.718 |
| 210836_x_at | PDE4D | phosphodiesterase 4D, cAMP-specific | −2.711 |
| | | (phosphodiesterase E3 dunce homolog, | |
| | | Drosophila) | |
| 240180_at | NA | NA | −2.703 |
| 208078_s_at | NA | NA | −2.691 |
| 202890_at | MAP7 | microtubule-associated protein 7 | −2.687 |
| 214602_at | COL4A4 | collagen, type IV, alpha 4 | −2.655 |
| 223721_s_at | DNAJC12 | DnaJ (Hsp40) homolog, subfamily C, | −2.652 |
| | | member 12 | |
| 209772_s_at | CD24 | CD24 molecule | −2.62 |
| 228825_at | PTGR1 | prostaglandin reductase 1 | −2.616 |
| 214234_s_at | NA | NA | −2.597 |
| 219194_at | SEMA4G | sema domain, immunoglobulin domain (Ig), | −2.581 |
| | | transmembrane domain (TM) and short | |
| | | cytoplasmic domain, (semaphorin) 4G | |
| 238441_at | PRKAA2 | protein kinase, AMP-activated, alpha 2 | −2.579 |
| | | catalytic subunit | |
| 218326_s_at | LGR4 | leucine-rich repeat-containing G protein- | −2.574 |
| | | coupled receptor 4 | |
| 208284_x_at | GGT1 | gamma-glutamyltransferase 1 | −2.549 |
| 239067_s_at | PANX2 | pannexin 2 | −2.546 |
| 240349_at | PRKAA2 | protein kinase, AMP-activated, alpha 2 | −2.534 |
| | | catalytic subunit | |
| 204567_s_at | ABCG1 | ATP-binding cassette, sub-family G | −2.525 |
| | | (WHITE), member 1 | |
| 235924_at | NA | NA | −2.504 |
| 227210_at | NA | NA | −2.501 |
| 211653_x_at | AKR1C2 | aldo-keto reductase family 1, member C2 | −2.497 |
| | | (dihydrodiol dehydrogenase 2; bile acid | |
| | | binding protein; 3-alpha hydroxysteroid | |
| | | dehydrogenase, type III) | |
| 225330_at | IGF1R | insulin-like growth factor 1 receptor | −2.497 |
| 1557147_a_at | NA | NA | −2.496 |
| 209160_at | AKR1C3 | aldo-keto reductase family 1, member C3 | −2.49 |
| | | (3-alpha hydroxysteroid dehydrogenase, | |
| | | type II) | |
| 209699_x_at | AKR1C2 | aldo-keto reductase family 1, member C2 | −2.477 |
| | | (dihydrodiol dehydrogenase 2; bile acid | |
| | | binding protein; 3-alpha hydroxysteroid | |
| | | dehydrogenase, type III) | |
| 211417_x_at | GGT1 | gamma-glutamyltransferase 1 | −2.459 |
| 241764_at | NA | NA | −2.458 |
| 214235_at | NA | NA | −2.451 |
| 39249_at | AQP3 | aquaporin 3 (Gill blood group) | −2.444 |
| 232921_at | KIAA1549 | KIAA1549 | −2.44 |
| 203192_at | ABCB6 | ATP-binding cassette, sub-family B | −2.428 |
| | | (MDR/TAP), member 6 | |
| 212327_at | LIMCH1 | LIM and calponin homology domains 1 | −2.423 |
| 212328_at | LIMCH1 | LIM and calponin homology domains 1 | −2.42 |
| 242794_at | MAML3 | mastermind-like 3 (Drosophila) | −2.416 |
| 227892_at | PRKAA2 | protein kinase, AMP-activated, alpha 2 | −2.401 |
| | | catalytic subunit | |
| 200731_s_at | PTP4A1 | protein tyrosine phosphatase type IVA, | −2.393 |
| | | member 1 | |
| 214434_at | HSPA12A | heat shock 70 kDa protein 12A | −2.384 |
| 39549_at | NPAS2 | neuronal PAS domain protein 2 | −2.374 |
| 213462_at | NPAS2 | neuronal PAS domain protein 2 | −2.348 |
| 213155_at | WSCD1 | WSC domain containing 1 | −2.341 |
| 218416_s_at | SLC48A1 | solute carrier family 48 (heme transporter), | −2.33 |
| | | member 1 | |
| 242917_at | RASGEF1A | RasGEF domain family, member 1A | −2.31 |
| 224937_at | PTGFRN | prostaglandin F2 receptor negative regulator | −2.308 |
| 205850_s_at | GABRB3 | gamma-aminobutyric acid (GABA) A | −2.307 |
| | | receptor, beta 3 | |
| 210263_at | KCNF1 | potassium voltage-gated channel, subfamily | −2.29 |
| | | F, member 1 | |
| 226448_at | FAM89A | family with sequence similarity 89, member A | −2.288 |
| 225056_at | SIPA1L2 | signal-induced proliferation-associated 1 | −2.278 |
| | | like 2 | |
| 210964_s_at | GYG2 | glycogenin 2 | −2.262 |
| 238537_at | NA | NA | −2.26 |
| 208322_s_at | ST3GAL1 | ST3 beta-galactoside alpha-2,3- | −2.256 |
| | | sialyltransferase 1 | |
| 218417_s_at | SLC48A1 | solute carrier family 48 (heme transporter), | −2.255 |
| | | member 1 | |
| 204151_x_at | AKR1C1 | aldo-keto reductase family 1, member C1 | −2.251 |
| | | (dihydrodiol dehydrogenase 1; 20-alpha (3- | |
| | | alpha)-hydroxysteroid dehydrogenase) | |
| 243586_at | NA | NA | −2.239 |
| 208650_s_at | CD24 | CD24 molecule | −2.229 |
| 223575_at | KIAA1549 | KIAA1549 | −2.229 |
| 210558_at | AKR1C4 | aldo-keto reductase family 1, member C4 | −2.228 |
| | | (chlordecone reductase; 3-alpha | |
| | | hydroxysteroid dehydrogenase, type I; | |
| | | dihydrodiol dehydrogenase 4) | |
| 216594_x_at | AKR1C1 | aldo-keto reductase family 1, member C1 | −2.224 |
| | | (dihydrodiol dehydrogenase 1; 20-alpha (3- | |
| | | alpha)-hydroxysteroid dehydrogenase) | |
| 219475_at | OSGIN1 | oxidative stress induced growth inhibitor 1 | −2.219 |
| 203615_x_at | SULT1A1 | sulfotransferase family, cytosolic, 1A, | −2.209 |
| | | phenol-preferring, member 1 | |
| 217590_s_at | TRPA1 | transient receptor potential cation channel, | −2.202 |
| | | subfamily A, member 1 | |
| 223633_s_at | BCAN | brevican | −2.199 |
| 200965_s_at | ABLIM1 | actin binding LIM protein 1 | −2.194 |
| 221802_s_at | KIAA1598 | KIAA1598 | −2.184 |
| 215695_s_at | GYG2 | glycogenin 2 | −2.178 |
| 65517_at | AP1M2 | adaptor-related protein complex 1, mu 2 | −2.176 |
| | | subunit | |
| 208651_x_at | CD24 | CD24 molecule | −2.175 |
| 242037_at | ASPH | aspartate beta-hydroxylase | −2.168 |
| 224950_at | PTGFRN | prostaglandin F2 receptor negative regulator | −2.166 |
| 215299_x_at | SULT1A1 | sulfotransferase family, cytosolic, 1A, | −2.161 |
| | | phenol-preferring, member 1 | |
| 226039_at | MGAT4A | mannosyl (alpha-1,3-)-glycoprotein beta- | −2.145 |
| | | 1,4-N-acetylglucosaminyltransferase, | |
| | | isozyme A | |
| 212651_at | RHOBTB1 | Rho-related BTB domain containing 1 | −2.131 |
| 200732_s_at | PTP4A1 | protein tyrosine phosphatase type IVA, | −2.099 |
| | | member 1 | |
| 209276_s_at | GLRX | glutaredoxin (thioltransferase) | −2.097 |
| 211302_s_at | PDE4B | phosphodiesterase 4B, cAMP-specific | −2.095 |
| | | (phosphodiesterase E4 dunce homolog, | |
| | | Drosophila) | |
| 223058_at | FAM107B | family with sequence similarity 107, | −2.095 |
| | | member B | |
| 215635_at | NA | NA | −2.078 |
| 204505_s_at | EPB49 | erythrocyte membrane protein band 4.9 | −2.062 |
| | | (dematin) | |
| 222496_s_at | RBM47 | RNA binding motif protein 47 | −2.059 |
| 48106_at | SLC48A1 | solute carrier family 48 (heme transporter), | −2.056 |
| | | member 1 | |
| 211382_s_at | TACC2 | transforming, acidic coiled-coil containing | −2.054 |
| | | protein 2 | |
| 218681_s_at | SDF2L1 | stromal cell-derived factor 2-like 1 | −2.04 |
| 216548_x_at | HMGB3L1 | high-mobility group box 3-like 1 | −2.031 |
| 234986_at | NA | NA | −2.02 |
| 223059_s_at | FAM107B | family with sequence similarity 107, | −2.017 |
| | | member B | |
| 202421_at | IGSF3 | immunoglobulin superfamily, member 3 | -2.009 |
| 225033_at | ST3GAL1 | ST3 beta-galactoside alpha-2,3- | -2.006 |
| | | sialyltransferase 1 | |
| 207709_at | PRKAA2 | protein kinase, AMP-activated, alpha 2 | −1.999 |
| | | catalytic subunit | |
| 238489_at | PRKAA2 | protein kinase, AMP-activated, alpha 2 | −1.996 |
| | | catalytic subunit | |
| 236140_at | GCLM | glutamate-cysteine ligase, modifier subunit | −1.994 |
| 218035_s_at | RBM47 | RNA binding motif protein 47 | −1.99 |
| 200730_s_at | PTP4A1 | protein tyrosine phosphatase type IVA, | −1.986 |
| | | member 1 | |
| 266_s_at | CD24 | CD24 molecule | −1.985 |
| 204058_at | ME1 | malic enzyme 1, NADPH-dependent, | −1.968 |
| | | cytosolic | |
| 1557689_at | NA | NA | −1.96 |
| 226886_at | GFPT1 | glutamine-fructose-6-phosphate | −1.959 |
| | | transaminase 1 | |
| 216379_x_at | CD24 | CD24 molecule | −1.925 |
| 209771_x_at | CD24 | CD24 molecule | −1.916 |
| 241459_at | LIMCH1 | LIM and calponin homology domains 1 | −1.916 |
| 39548_at | NPAS2 | neuronal PAS domain protein 2 | −1.911 |
| 206662_at | GLRX | glutaredoxin (thioltransferase) | −1.904 |
| 221245_s_at | FZD5 | frizzled homolog 5 (Drosophila) | −1.893 |
| 207178_s_at | FRK | fyn-related kinase | −1.89 |
| 203343_at | UGDH | UDP-glucose dehydrogenase | −1.861 |
| 209607_x_at | NA | NA | −1.856 |
| 212325_at | LIMCH1 | LIM and calponin homology domains 1 | −1.845 |
| 226055_at | ARRDC2 | arrestin domain containing 2 | −1.838 |
| 226653_at | MARK1 | MAP/microtubule affinity-regulating kinase 1 | −1.809 |
| 205459_s_at | NPAS2 | neuronal PAS domain protein 2 | −1.799 |
| 226003_at | KIF21A | kinesin family member 21A | −1.756 |
| 229002_at | FAM69B | family with sequence similarity 69, member B | −1.732 |
| 202206_at | ARL4C | ADP-ribosylation factor-like 4C | 1.504 |
| 231017_at | STK11 | serine/threonine kinase 11 | 1.514 |
| 1554769_at | ZNF785 | zinc finger protein 785 | 1.594 |
| 201105_at | LGALS1 | lectin, galactoside-binding, soluble, 1 | 1.603 |
| 218154_at | GSDMD | gasdermin D | 1.604 |
| 211799_x_at | HLA-C | major histocompatibility complex, class I, C | 1.633 |
| 1553193_at | ZNF441 | zinc finger protein 441 | 1.669 |
| 201109_s_at | THBS1 | thrombospondin 1 | 1.672 |
| 221903_s_at | CYLD | cylindromatosis (turban tumor syndrome) | 1.682 |
| 242028_at | NA | NA | 1.689 |
| 201110_s_at | THBS1 | thrombospondin 1 | 1.703 |
| 211962_s_at | ZFP36L1 | zinc finger protein 36, C3H type-like 1 | 1.707 |
| 225328_at | NA | NA | 1.719 |
| 238940_at | KLF12 | Kruppel-like factor 12 | 1.739 |
| 231215_at | NA | NA | 1.753 |
| 221534_at | C11orf68 | chromosome 11 open reading frame 68 | 1.766 |
| 228213_at | H2AFJ | H2A histone family, member J | 1.779 |
| 235171_at | NA | NA | 1.81 |
| 214860_at | SLC9A7 | solute carrier family 9 (sodium/hydrogen | 1.836 |
| | | exchanger), member 7 | |
| 216526_x_at | HLA-C | major histocompatibility complex, class I, C | 1.839 |
| 201466_s_at | JUN | jun oncogene | 1.848 |
| 228394_at | STK10 | serine/threonine kinase 10 | 1.851 |
| 1555675_at | BLID | BH3-like motif containing, cell death | 1.886 |
| | | inducer | |
| 239426_at | SLC2A8 | solute carrier family 2 (facilitated glucose | 1.888 |
| | | transporter), member 8 | |
| 208812_x_at | HLA-C | major histocompatibility complex, class I, C | 1.899 |
| 242458_at | RALGPS2 | Ral GEF with PH domain and SH3 binding | 1.902 |
| | | motif 2 | |
| 229224_x_at | LOC643085 | hypothetical LOC643085 | 1.906 |
| 230536_at | PBX4 | pre-B-cell leukemia homeobox 4 | 1.913 |
| 1553247_a_at | ZNF709 | zinc finger protein 709 | 1.918 |
| 1552671_a_at | SLC9A7 | solute carrier family 9 (sodium/hydrogen | 1.918 |
| | | exchanger), member 7 | |
| 226550_at | NA | NA | 1.939 |
| 230112_at | 4-Mar | membrane-associated ring finger (C3HC4) 4 | 1.955 |
| 228121_at | TGFB2 | transforming growth factor, beta 2 | 1.956 |
| 211165_x_at | EPHB2 | EPH receptor B2 | 1.985 |
| 209651_at | TGFB1I1 | transforming growth factor beta 1 induced | 1.989 |
| | | transcript 1 | |
| 219427_at | FAT4 | FAT tumor suppressor homolog 4 | 1.99 |
| | | (Drosophila) | |
| 208025_s_at | HMGA2 | high mobility group AT-hook 2 | 1.996 |
| 214459_x_at | HLA-C | major histocompatibility complex, class I, C | 1.996 |
| 203047_at | STK10 | serine/threonine kinase 10 | 1.996 |
| 203988_s_at | FUT8 | fucosyltransferase 8 (alpha (1,6) | 1.997 |
| | | fucosyltransferase) | |
| 227503_at | NA | NA | 2.005 |
| 211529_x_at | HLA-G | major histocompatibility complex, class I, G | 2.012 |
| 209304_x_at | GADD45B | growth arrest and DNA-damage-inducible, | 2.014 |
| | | beta | |
| 211528_x_at | HLA-G | major histocompatibility complex, class I, G | 2.031 |
| 1558626_at | NA | NA | 2.037 |
| 1558105_a_at | NA | NA | 2.044 |
| 201462_at | SCRN1 | secernin 1 | 2.047 |
| 208729_x_at | HLA-B | major histocompatibility complex, class I, B | 2.062 |
| 207574_s_at | GADD45B | growth arrest and DNA-damage-inducible, | 2.064 |
| | | beta | |
| 37547_at | BBS9 | Bardet-Biedl syndrome 9 | 2.069 |
| 225388_at | TSPAN5 | tetraspanin 5 | 2.072 |
| 210875_s_at | ZEB1 | zinc finger E-box binding homeobox 1 | 2.077 |
| 232247_at | ZNF502 | zinc finger protein 502 | 2.077 |
| 209140_x_at | HLA-B | major histocompatibility complex, class I, B | 2.08 |
| 227088_at | PDESA | phosphodiesterase 5A, cGMP-specific | 2.083 |
| 229014_at | F1142709 | hypothetical LOC441094 | 2.096 |
| 227489_at | SMURF2 | SMAD specific E3 ubiquitin protein ligase 2 | 2.102 |
| 244180_at | ZNF793 | zinc finger protein 793 | 2.114 |
| 239105_at | NA | NA | 2.116 |
| 210655_s_at | NA | NA | 2.119 |
| 232774_x_at | ZIK1 | zinc finger protein interacting with K | 2.123 |
| | | protein 1 homolog (mouse) | |
| 211911_x_at | HLA-B | major histocompatibility complex, class I, B | 2.125 |
| 212607_at | AKT3 | v-akt murine thymoma viral oncogene | 2.136 |
| | | homolog 3 (protein kinase B, gamma) | |
| 244241_x_at | NA | NA | 2.137 |
| 209305_s_at | GADD45B | growth arrest and DNA-damage-inducible, | 2.14 |
| | | beta | |
| 1558391_s_at | ZNF599 | zinc finger protein 599 | 2.164 |
| 217624_at | PDAP1 | PDGFA associated protein 1 | 2.182 |
| 202084_s_at | SEC14L1 | SEC14-like 1 (S. cerevisiae) | 2.187 |
| 225524_at | ANTXR2 | anthrax toxin receptor 2 | 2.2 |
| 231879_at | COL12A1 | collagen, type XII, alpha 1 | 2.204 |
| 236044_at | PPAPDC1A | phosphatidic acid phosphatase type 2 | 2.224 |
| | | domain containing 1A | |
| 219765_at | ZNF329 | zinc finger protein 329 | 2.238 |
| 1558683_a_at | HMGA2 | high mobility group AT-hook 2 | 2.247 |
| 218718_at | PDGFC | platelet derived growth factor C | 2.249 |
| 214995_s_at | NA | NA | 2.254 |
| 241826_x_at | ZNF738 | zinc finger protein 738 | 2.257 |
| 219523_s_at | ODZ3 | odz, odd Oz/ten-m homolog 3 (Drosophila) | 2.262 |
| 205596_s_at | SMURF2 | SMAD specific E3 ubiquitin protein ligase 2 | 2.327 |
| 218986_s_at | DDX60 | DEAD (Asp-Glu-Ala-Asp) box polypeptide 60 | 2.336 |
| 204292_x_at | STK11 | serine/threonine kinase 11 | 2.348 |
| 230820_at | NA | NA | 2.354 |
| 212985_at | APBB2 | amyloid beta (A4) precursor protein- | 2.356 |
| | | binding, family B, member 2 | |
| 202082_s_at | SEC14L1 | SEC14-like 1 (S. cerevisiae) | 2.363 |
| 210001_s_at | SOCS1 | suppressor of cytokine signaling 1 | 2.371 |
| 206659_at | NA | NA | 2.374 |
| 235027_at | NA | NA | 2.38 |
| 228208_x_at | ZNF354C | zinc finger protein 354C | 2.391 |
| 208790 _s_at | PTRF | polymerase I and transcript release factor | 2.406 |
| 239761_at | GCNT1 | glucosaminyl (N-acetyl) transferase 1, core | 2.414 |
| | | 2 (beta-1,6-N- | |
| | | acetylglucosaminyltransferase) | |
| 218273_s_at | PDP1 | pyruvate dehyrogenase phosphatase | 2.415 |
| | | catalytic subunit 1 | |
| 1562386_s_at | ZNF501 | zinc finger protein 501 | 2.416 |
| 204897_at | PTGER4 | prostaglandin E receptor 4 (subtype EP4) | 2.435 |
| 213325_at | PVRL3 | poliovirus receptor-related 3 | 2.437 |
| 222572_at | PDP1 | pyruvate dehyrogenase phosphatase | 2.441 |
| | | catalytic subunit 1 | |
| 205505_at | GCNT1 | glucosaminyl (N-acetyl) transferase 1, core | 2.461 |
| | | 2 (beta-1,6-N- | |
| | | acetylglucosaminyltransferase) | |
| 222880_at | AKT3 | v-akt murine thymoma viral oncogene | 2.463 |
| | | homolog 3 (protein kinase B, gamma) | |
| 239669_at | NA | NA | 2.468 |
| 229533_x_at | ZNF680 | zinc finger protein 680 | 2.479 |
| 238149_at | ZNF818P | zinc finger protein 818 pseudogene | 2.481 |
| 201649_at | UBE2L6 | ubiquitin-conjugating enzyme E2L 6 | 2.49 |
| 239204_at | ZNF75A | zinc finger protein 75a | 2.495 |
| 233002_at | PPP4R4 | protein phosphatase 4, regulatory subunit 4 | 2.529 |
| 238944_at | ZNF404 | zinc finger protein 404 | 2.546 |
| 203989_x_at | F2R | coagulation factor II (thrombin) receptor | 2.552 |
| 1567224_at | HMGA2 | high mobility group AT-hook 2 | 2.558 |
| 1562415_a_at | SPOCD1 | SPOC domain containing 1 | 2.566 |
| 232020_at | SMURF2 | SMAD specific E3 ubiquitin protein ligase 2 | 2.584 |
| 200665_s_at | SPARC | secreted protein, acidic, cysteine-rich | 2.6 |
| | | (osteonectin) | |
| 218656_s_at | LHFP | lipoma HMGIC fusion partner | 2.606 |
| 230345_at | SEMA7A | semaphorin 7A, GPI membrane anchor | 2.613 |
| | | (John Milton Hagen blood group) | |
| 1561633_at | HMGA2 | high mobility group AT-hook 2 | 2.631 |
| 228054_at | TMEM44 | transmembrane protein 44 | 2.631 |
| 205514_at | ZNF415 | zinc finger protein 415 | 2.633 |
| 209505_at | NR2F1 | nuclear receptor subfamily 2, group F, | 2.657 |
| | | member 1 | |
| 228843_at | NA | NA | 2.665 |
| 201325_s_at | EMP1 | epithelial membrane protein 1 | 2.681 |
| 202686_s_at | AXL | AXL receptor tyrosine kinase | 2.691 |
| 201324_at | EMP1 | epithelial membrane protein 1 | 2.724 |
| 206557_at | ZNF702P | zinc finger protein 702 pseudogene | 2.738 |
| 231930_at | ELMOD1 | ELMO/CED-12 domain containing 1 | 2.745 |
| 228278_at | NFIX | nuclear factor I/X (CCAAT-binding | 2.765 |
| | | transcription factor) | |
| 227828_s_at | FAM176A | family with sequence similarity 176, | 2.783 |
| | | member A | |
| 220738_s_at | RPS6KA6 | ribosomal protein S6 kinase, 90 kDa, | 2.788 |
| | | polypeptide 6 | |
| 207156_at | HIST1H2AG | histone cluster 1, H2ag | 2.821 |
| 224833_at | ETS1 | v-ets erythroblastosis virus E26 oncogene | 2.838 |
| | | homolog 1 (avian) | |
| 1569470_a_at | FRMD5 | FERM domain containing 5 | 2.841 |
| 235417_at | SPOCD1 | SPOC domain containing 1 | 2.844 |
| 202083_s_at | SEC14L1 | SEC14-like 1 (S. cerevisiae) | 2.852 |
| 222571_at | ST6GALNAC6 | ST6 (alpha-N-acetyl-neuraminy1-2,3-beta- | 2.859 |
| | | galactosyl-1,3)-N-acetylgalactosaminide | |
| | | alpha-2,6-sialyltransferase 6 | |
| 244551_at | NA | NA | 2.893 |
| 224822_at | DLC1 | deleted in liver cancer 1 | 2.895 |
| 207068_at | ZFP37 | zinc finger protein 37 homolog (mouse) | 2.901 |
| 236847_at | C19orf18 | chromosome 19 open reading frame 18 | 2.911 |
| 217999_s_at | PHLDA1 | pleckstrin homology-like domain, family A, | 2.921 |
| | | member 1 | |
| 222719_s_at | PDGFC | platelet derived growth factor C | 2.948 |
| 209890_at | TSPAN5 | tetraspanin 5 | 2.996 |
| 238050_at | ANTXR2 | anthrax toxin receptor 2 | 3.015 |
| 225387_at | TSPAN5 | tetraspanin 5 | 3.021 |
| 208081_s_at | ZNF442 | zinc finger protein 442 | 3.046 |
| 230831_at | FRMD5 | FERM domain containing 5 | 3.057 |
| 209156_s_at | COL6A2 | collagen, type VI, alpha 2 | 3.088 |
| 240407_at | LOC100126784 | hypothetical LOC100126784 | 3.09 |
| 228950_s_at | GPR177 | G protein-coupled receptor 177 | 3.109 |
| 221958_s_at | GPR177 | G protein-coupled receptor 177 | 3.125 |
| 228368_at | ARHGAP20 | Rho GTPase activating protein 20 | 3.236 |
| 221087_s_at | APOL3 | apolipoprotein L, 3 | 3.27 |
| 232231_at | RUNX2 | runt-related transcription factor 2 | 3.292 |
| 204823_at | NAV3 | neuron navigator 3 | 3.293 |
| 218691_s_at | PDLIM4 | PDZ and LIM domain 4 | 3.35 |
| 229059_at | NA | NA | 3.352 |
| 1557636_a_at | C7orf57 | chromosome 7 open reading frame 57 | 3.352 |
| 231766_s_at | COL12A1 | collagen, type XII, alpha 1 | 3.362 |
| 1552658_a_at | NAV3 | neuron navigator 3 | 3.378 |
| 229430_at | NA | NA | 3.44 |
| 235944_at | HMCN1 | hemicentin 1 | 3.44 |
| 228949_at | GPR177 | G protein-coupled receptor 177 | 3.444 |
| 204415_at | IFI6 | interferon, alpha-inducible protein 6 | 3.521 |
| 206170_at | ADRB2 | adrenergic, beta-2-, receptor, surface | 3.533 |
| 1552309_a_at | NEXN | nexilin (F actin binding protein) | 3.534 |
| 218312_s_at | ZSCAN18 | zinc finger and SCAN domain containing 18 | 3.547 |
| 223794_at | ARMC4 | armadillo repeat containing 4 | 3.565 |
| 230968_at | NA | NA | 3.696 |
| 206230_at | LHX1 | LIM homeobox 1 | 3.852 |
| 239043_at | ZNF404 | zinc finger protein 404 | 3.869 |
| 202411_at | IF127 | interferon, alpha-inducible protein 27 | 3.906 |
| 231470_at | NA | NA | 3.994 |
| 226103_at | NEXN | nexilin (F actin binding protein) | 4.055 |
| 226218_at | IL7R | interleukin 7 receptor | 4.061 |
| 213338_at | TMEM158 | transmembrane protein 158 | 4.302 |
| 231728_at | CAPS | calcyphosine | 4.309 |
| 205798_at | IL7R | interleukin 7 receptor | 4.332 |
| 214175_x_at | PDLIM4 | PDZ and LIM domain 4 | 4.526 |
| 211564_s_at | PDLIM4 | PDZ and LIM domain 4 | 4.593 |
| 239250_at | ZNF542 | zinc finger protein 542 | 4.607 |
| 233504_at | C9orf84 | chromosome 9 open reading frame 84 | 4.71 |
| 243818_at | SFTA1P | surfactant associated 1 (pseudogene) | 4.772 |
| 219885_at | SLFN12 | schlafen family member 12 | 5.084 |
| 215446_s_at | LOX | lysyl oxidase | 5.089 |
| 213139_at | SNAI2 | snail homolog 2 (Drosophila) | 5.214 |
| 204298_s_at | LOX | lysyl oxidase | 5.555 |
| 203153_at | IFIT1 | interferon-induced protein with | 5.74 |
| | | tetratricopeptide repeats 1 | |
| 206421_s_at | SERPINB7 | serpin peptidase inhibitor, clade B | 5.838 |
| | | (ovalbumin), member 7 | |
| 204205_at | APOBEC3G | apolipoprotein B mRNA editing enzyme, | 6.054 |
| | | catalytic polypeptide-like 3G | |
| 206157_at | PTX3 | pentraxin-related gene, rapidly induced by | 6.193 |
| | | IL-1 beta | |
| 1569039_s_at | ZNF677 | zinc finger protein 677 | 6.8 |
| 244552_at | ZNF788 | zinc finger family member 788 | 7.004 |
| 228974_at | NA | NA | 7.033 |
| 228617_at | XAF1 | XIAP associated factor 1 | 7.056 |
| 231098_at | NA | NA | 7.498 |
| 203435_s_at | MME | membrane metallo-endopeptidase | 7.574 |
| 202202_s_at | LAMA4 | laminin, alpha 4 | 7.834 |
| 1560562_a_at | ZNF677 | zinc finger protein 677 | 8.104 |
| 203434_s_at | MME | membrane metallo-endopeptidase | 8.713 |
| 1555759_a_at | CCL5 | chemokine (C-C motif) ligand 5 | 9.405 |
| 227655_at | SNORD123 | small nucleolar RNA, C/D box 123 | 9.436 |
| 209619_at | CD74 | CD74 molecule, major histocompatibility | 10.572 |
| | | complex, class II invariant chain | |
| 235236_at | LOC100131897 | Uncharacterized protein LOC 100131897 | 13.196 |
| 231729_s_at | CAPS | calcyphosine | 15.222 |
| 211518_s_at | BMP4 | bone morphogenetic protein 4 | 17.634 |
| 1555673_at | NA | NA | 20.578 |
| 1405_i_at | CCL5 | chemokine (C-C motif) ligand 5 | 23 .398 |
| 231867_at | ODZ2 | odz, odd Oz/ten-m homolog 2 (Drosophila) | 29.634 |
| 209396_s_at | CHI3L1 | chitinase 3-like 1 (cartilage glycoprotein-39) | 42.124 |
| 209395_at | CHI3L1 | chitinase 3-like 1 (cartilage glycoprotein-39) | 48.846 |
| 242206_at | NA | NA | 70.713 |

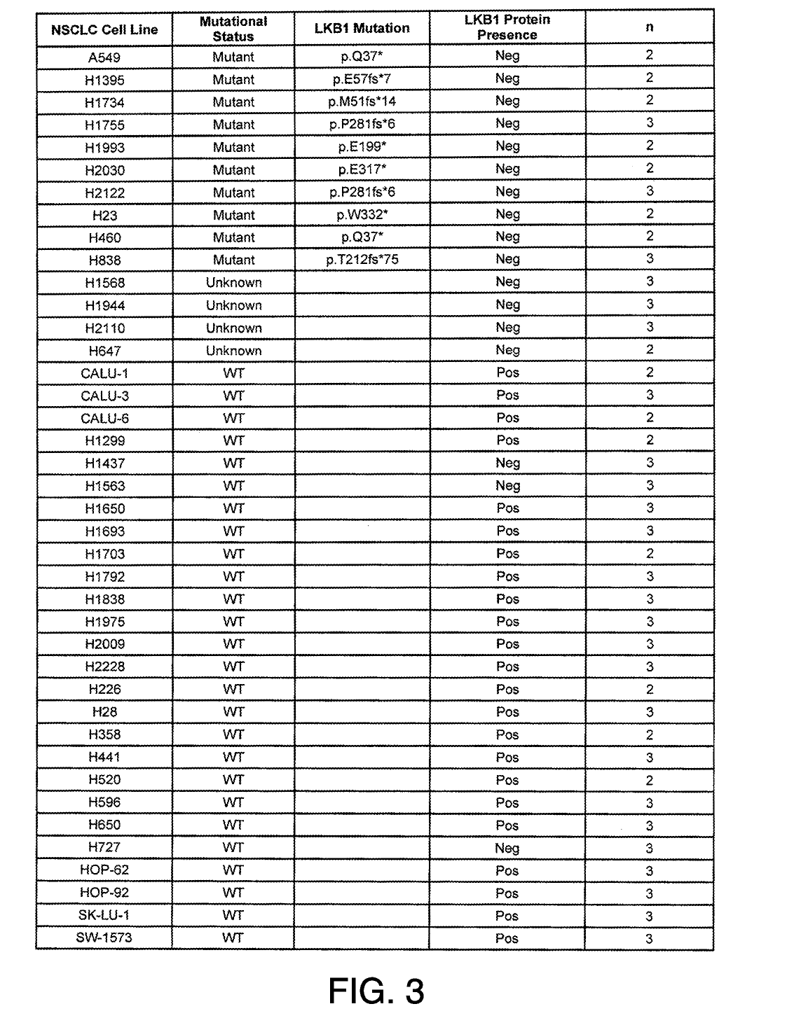

Fold Change values were computed by dividing the average gene expression for LKB1 positive cell lines (wild type) with the average gene expression of LKB1 negative cell lines (see FIG. 3 for positive and negative LKB1 cell lines). When the fold change is <1, the negative reciprocal of the original value is taken as the final fold change. A negative Fold Change value therefore means that LKB1 positive cell lines have a lower expression than LKB1 negative cell lines.

### 5.3 TOR Kinase Inhibitors

The compounds provided herein are generally referred to as TOR kinase inhibitors or “TORKi.” In a specific embodiment, the TORKi do not include rapamycin or rapamycin analogs (rapalogs). In certain embodiments, compounds provided herein are also DNA-PK inhibitors or “DNA-PKi.”

In one embodiment, the TOR kinase inhibitors include compounds having the following formula (I):

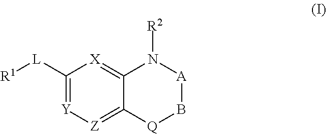

and pharmaceutically acceptable salts, clathrates, solvates, stereoisomers, tautomers, and prodrugs thereof, wherein:

X, Y and Z are at each occurrence independently N or CR³, wherein at least one of X, Y and Z is N and at least one of X, Y and Z is CR³;

-A-B-Q- taken together form —CHR⁴C(O)NH—, —C(O)CHR⁴NH—, —C(O)NH—, —CH₂C(O)O—, —C(O)CH₂O—, —C(O)O— or C(O)NR³;

L is a direct bond, NH or O;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl;

R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl;

R³ is H, substituted or unsubstituted C₁₋₈alkyl, substituted or unsubstituted aryl, substituted or unsubstituted heteroaryl, substituted or unsubstituted cycloalkyl, substituted or unsubstituted heterocyclylalkyl, —NHR⁴ or —N(R⁴)₂; and

R⁴ is at each occurrence independently substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (I) are those wherein -A-B-Q- taken together form —CH₂C(O)NH—.

In another embodiment, the TOR kinase inhibitors of formula (I) are those wherein -A-B-Q- taken together form —C(O)CH₂NH—.

In another embodiment, the TOR kinase inhibitors of formula (I) are those wherein -A-B-Q- taken together form —C(O)NH—.

In another embodiment, the TOR kinase inhibitors of formula (I) are those wherein -A-B-Q- taken together form —CH₂C(O)O—.

In another embodiment, the TOR kinase inhibitors of formula (I) are those wherein -A-B-Q- taken together form —C(O)CH₂O—.

In another embodiment, the TOR kinase inhibitors of formula (I) are those wherein -A-B-Q- taken together form —C(O)O—.

In another embodiment, the TOR kinase inhibitors of formula (I) are those wherein -A-B-Q- taken together form —C(O)NR³—.

In another embodiment, the TOR kinase inhibitors of formula (I) are those wherein Y is CR³.

In another embodiment, the TOR kinase inhibitors of formula (I) are those wherein X and Z are N and Y is CR³.

In another embodiment, the TOR kinase inhibitors of formula (I) are those wherein X and Z are N and Y is CH.

In another embodiment, the TOR kinase inhibitors of formula (I) are those wherein X and Z are CH and Y is N.

In another embodiment, the TOR kinase inhibitors of formula (I) are those wherein Y and Z are CH and X is N.

In another embodiment, the TOR kinase inhibitors of formula (I) are those wherein X and Y are CH and Z is N.

In another embodiment, the TOR kinase inhibitors of formula (I) are those wherein R¹ is substituted aryl, such as substituted phenyl.

In another embodiment, the TOR kinase inhibitors of formula (I) are those wherein R¹ is substituted or unsubstituted aryl, such as substituted or unsubstituted phenyl or substituted or unsubstituted naphthyl.

In another embodiment, the TOR kinase inhibitors of formula (I) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (I) are those wherein R¹ is H.

In another embodiment, the TOR kinase inhibitors of formula (I) are those wherein R² is substituted C₁₋₈alkyl.

In another embodiment, the TOR kinase inhibitors of formula (I) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (I) are those wherein R² is methyl or ethyl substituted with substituted or unsubstituted cycloalkyl, or substituted or unsubstituted heterocyclylalkyl.

In another embodiment, the TOR kinase inhibitors of formula (I) are those wherein R² is C₁₋₄alkyl substituted with substituted or unsubstituted cycloalkyl, or substituted or unsubstituted heterocyclylalkyl.

In another embodiment, the TOR kinase inhibitors of formula (I) are those wherein R² is substituted or unsubstituted cycloalkyl or substituted or unsubstituted heterocyclylalkyl.

In another embodiment, the TOR kinase inhibitors of formula (I) are those wherein R² is substituted or unsubstituted aryl, such as substituted or unsubstituted phenyl.

In another embodiment, the TOR kinase inhibitors of formula (I) are those wherein R² is H.

In another embodiment, the TOR kinase inhibitors of formula (I) are those wherein L is a direct bond.

In another embodiment, the TOR kinase inhibitors of formula (I) are those wherein -A-B-Q- taken together form —C(O)NH—, X and Z are N and Y is CH, R¹ is substituted or unsubstituted aryl or substituted or unsubstituted heteroaryl, L is a direct bond, and R² is substituted or unsubstituted C₁₋₈alkyl.

In another embodiment, the TOR kinase inhibitors of formula (I) are those wherein -A-B-Q- taken together form —C(O)NH—, X and Z are N and Y is CH, R¹ is substituted or unsubstituted aryl, L is a direct bond, and R² is substituted or unsubstituted C₁₋₈alkyl.

In another embodiment, the TOR kinase inhibitors of formula (I) are those wherein -A-B-Q- taken together form —C(O)NH—, X and Z are N and Y is CH, R¹ is substituted or unsubstituted aryl, and R² is C₁₋₈alkyl substituted with one or more substituents selected from alkoxy, amino, hydroxy, cycloalkyl, or heterocyclylalkyl.

In another embodiment, the TOR kinase inhibitors of formula (I) are those wherein -A-B-Q- taken together form —C(O)NH—, X and Z are N and Y is CH, R¹ is substituted or unsubstituted aryl, and R² is substituted or unsubstituted cycloalkyl, or substituted or unsubstituted heterocyclylalkyl.

In another embodiment, the TOR kinase inhibitors of formula (I) are those wherein -A-B-Q- taken together form —C(O)NH—, X and Z are N and Y is CH, R¹ is substituted phenyl, L is a direct bond, and R² is substituted C₁₋₈alkyl.

In another embodiment, the TOR kinase inhibitors of formula (I) do not include compounds wherein X and Z are both N and Y is CH, -A-B-Q- is —C(O)NH—, L is a direct bond, R¹ is substituted or unsubstituted aryl or substituted or unsubstituted heteroaryl, and R² is C₁₋₈alkyl substituted with substituted or unsubstituted aryl or substituted or unsubstituted heteroaryl.

In another embodiment, the TOR kinase inhibitors of formula (I) do not include compounds wherein X and Z are both N and Y is CH, -A-B-Q- is —C(O)NH—, L is a direct bond, R¹ is phenyl, naphthyl, indanyl or biphenyl, each of which may be optionally substituted with one or more substituents independently selected from the group consisting substituted or unsubstituted C₁₋₈alkyl, substituted or unsubstituted C₂₋₈alkenyl, substituted or unsubstituted aryl, substituted or unsubstituted cycloalkyl or substituted or unsubstituted heterocyclylalkyl.

In another embodiment, the TOR kinase inhibitors of formula (I) do not include compounds wherein X and Z are both N and Y is CH, -A-B-Q- is —C(O)NH—, L is a direct bond, R¹ is phenyl, naphthyl or biphenyl, each of which may be optionally substituted with one or more substituents each independently selected from the group consisting of C₁₋₄alkyl, amino, aminoC₁₋₁₂alkyl, halogen, hydroxy, hydroxyC₁₋₄alkyl, C₁₋₄alkyloxyC₁₋₄alkyl, —CF₃, C₁₋₁₂alkoxy, aryloxy, arylC₁₋₁₂alkoxy, —CN, —OCF₃, —COR_{g}, —COOR_{g}, —CONR_{g}Rₕ, —NR_{g}CORₕ, —SO₂R_{g}, —SO₃R_{g} or —SO₂NR_{g}Rₕ, wherein each R_{g} and Rₕ are independently selected from the group consisting of hydrogen, C₁₋₄alkyl, C₃₋₆cycloalkyl, aryl, arylC₁₋₆alkyl, heteroaryl or heteroarylC₁₋₆alkyl; or A is a 5- to 6-membered monocyclic heteroaromatic ring having from one, two, three or four heteroatoms independently selected from the group consisting of N, O and S, that monocyclic heteroaromatic ring may be optionally substituted with one or more substituents each independently selected from the group consisting of C₁₋₆alkyl, amino, aminoC₁₋₁₂alkyl, halogen, hydroxy, hydroxyC₁₋₄alkyl, C₁₋₄alkyloxyC₁₋₄alkyl, C₁₋₁₂alkoxy, aryloxy, aryl C₁₋₁₂alkoxy, —CN, —CF₃, —OCF₃, —CORᵢ, —COORᵢ, —CONRᵢRⱼ, —NRᵢCORⱼ, —NRᵢSO₂Rⱼ, —SO₂Rᵢ, —SO₃Rᵢ, or —SO₂NRᵢRⱼ, wherein each Rᵢ and Rⱼ are independently selected from the group consisting of hydrogen, C₁₋₄ alkyl, C₃₋₆cycloalkyl, aryl, arylC₁₋₆alkyl, heteroaryl or heteroarylC₁₋₆alkyl; or A is a 8- to 10 membered bicyclic heteroaromatic ring from one, two, three or four heteroatoms selected from the group consisting of N, O and S, and may be optionally substituted with one, two or three substituents each independently selected from the group consisting of C₁₋₆alkyl, amino, aminoC₁₋₆alkyl, halogen, hydroxy, hydroxyC₁₋₄alkyl, C₁₋₄alkyloxyC₁₋₄alkyl, C₁₋₄alkoxy, aryloxy, aryl C₁₋₁₂alkoxy, —CN, —CF₃, —OCF₃, —CORₖ, —COORₖ, —CONRₖRₗ, —NRₖCORₗ, —NRkSO₂Rₗ, —SO₂Rₖ, —SO₃Rₖ or —SO₂NRₖRₗ, wherein each Rₖ and Rₗ are independently selected from the group consisting of hydrogen, C₁₋₄ alkyl, C₃₋₆ cycloalkyl, aryl, arylC₁₋₆alkyl, heteroaryl or heteroarylC₁₋₆alkyl, and R² is C₁₋₈alkyl substituted with substituted or unsubstituted aryl or substituted or unsubstituted heteroaryl.

In another embodiment, the TOR kinase inhibitors of formula (I) do not include compounds wherein X and Y are both N and Z is CH, -A-B-Q- is —C(O)NH—, L is a direct bond, R¹ is substituted or unsubstituted phenyl or substituted or unsubstituted heteroaryl, and R² is substituted or unsubstituted methyl, unsubstituted ethyl, unsubstituted propyl, or an acetamide.

In another embodiment, the TOR kinase inhibitors of formula (I) do not include compounds wherein X and Y are both N and Z is CH, -A-B-Q- is —C(O)NH—, L is a direct bond, R¹ is substituted or unsubstituted phenyl or substituted or unsubstituted heteroaryl, and R² is an acetamide.

In another embodiment, the TOR kinase inhibitors of formula (I) do not include compounds wherein X is N and Y and Z are both CH, -A-B-Q- is —C(O)NH—, L is a direct bond, R¹ is a (2,5′-Bi-1H-benzimidazole)-5-carboxamide, and R² is H.

In another embodiment, the TOR kinase inhibitors of formula (I) do not include compounds wherein one of X and Z is CH and the other is N, Y is CH, -A-B-Q- is —C(O)NH—, L is a direct bond, R¹ is unsubstituted pyridine, and R² is H, methyl or substituted ethyl.

In another embodiment, the TOR kinase inhibitors of formula (I) do not include compounds wherein X and Z are both N and Y is CH, -A-B-Q- is —C(O)NH—, R¹ is H, C₁₋₈alkyl, C₂₋₈alkenyl, aryl or cycloalkyl, and L is NH.

In another embodiment, the TOR kinase inhibitors of formula (I) do not include compounds wherein X and Z are both N and Y is CH, -A-B-Q- is —C(O)NR³—, R² is H, substituted or unsubstituted C₁₋₈alkyl, substituted or unsubstituted phenyl, substituted or unsubstituted cycloalkyl, or substituted or unsubstituted heterocyclylalkyl, and L is NH.

In another embodiment, the TOR kinase inhibitors of formula (I) do not include compounds wherein R¹ is a substituted or unsubstituted oxazolidinone.

In another embodiment, the TOR kinase inhibitors of formula (I) do not include one or more of the following compounds: 1,7-dihydro-2-phenyl-8H-Purin-8-one, 1,2-dihydro-3-phenyl-6H-Imidazo[4,5-e]-1,2,4-triazin-6-one, 1,3-dihydro-6-(4-pyridinyl)-2H-Imidazo[4,5-b ]pyridin-2-one, 6-(1,3-benzodioxol-5-yl)-1,3-dihydro-1-[(1S)-1-phenylethyl]-2H-Imidazo[4,5-b]pyrazin-2-one, 3-[2,3-dihydro-2-oxo-3-(4-pyridinylmethyl)-1H-imidazo[4,5-b]pyrazin-5-yl]-Benzamide, 1-[2-(dimethylamino)ethyl]-1,3-dihydro-6-(3,4,5-trimethoxyphenyl)-2H-Imidazo[4,5-b]pyrazin-2-one, N-[5-(1,1-dimethylethyl)-2-methoxyphenyl]-N′-[4-(1,2,3,4-tetrahydro-2-oxopyrido[2,3-b]pyrazin-7-yl)-1-naphthalenyl]-Urea, N-[4-(2,3-dihydro-2-oxo-1H-imidazo[4,5-b]pyridin-6-yl)-1-naphthalenyl]-N′-[5-(1,1-dimethylethyl)-2-methoxyphenyl]-Urea, 1,3-dihydro-5-phenyl-2H-Imidazo[4,5-b]pyrazin-2-one, 1,3-dihydro-5-phenoxy-2H-Imidazo[4,5-b]pyridin-2-one, 1,3-dihydro-1-methyl-6-phenyl-2H-Imidazo[4,5-b]pyridin-2-one, 1,3-dihydro-5-(1H-imidazol-1-yl) 2H-Imidazo[4,5-b]pyridin-2-one, 6-(2,3-dihydro-2-oxo-1H-imidazo[4,5-b]pyridin-6-yl)-8-methyl-2(1H)-Quinolinone and 7,8-dihydro-8-oxo-2-phenyl-9H-purine-9-acetic acid.

In one embodiment, the TOR kinase inhibitors include compounds having the following formula (Ia):

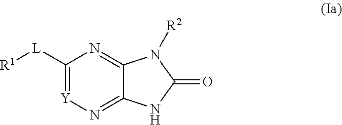

and pharmaceutically acceptable salts, clathrates, solvates, stereoisomers, tautomers, and prodrugs thereof, wherein:

L is a direct bond, NH or O;

Y is N or CR³;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl;

R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl;

R³ is H, substituted or unsubstituted C₁₋₈alkyl, substituted or unsubstituted aryl, substituted or unsubstituted heteroaryl, substituted or unsubstituted cycloalkyl, substituted or unsubstituted heterocyclylalkyl, —NHR⁴ or —N(R⁴)₂; and

R⁴ is at each occurrence independently substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (Ia) are those wherein R¹ is substituted aryl, such as substituted phenyl.

In another embodiment, the TOR kinase inhibitors of formula (Ia) are those wherein R¹ is substituted or unsubstituted aryl, such as substituted or unsubstituted phenyl or substituted or unsubstituted naphthyl.

In another embodiment, the TOR kinase inhibitors of formula (Ia) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (Ia) are those wherein R¹ is H.

In another embodiment, the TOR kinase inhibitors of formula (Ia) are those wherein R² is substituted C₁₋₈alkyl.

In another embodiment, the TOR kinase inhibitors of formula (Ia) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (Ia) are those wherein R² is substituted or unsubstituted cycloalkyl or substituted or unsubstituted heterocyclylalkyl.

In another embodiment, the TOR kinase inhibitors of formula (Ia) are those wherein R² is substituted or unsubstituted aryl, such as substituted or unsubstituted phenyl.

In another embodiment, the TOR kinase inhibitors of formula (Ia) are those wherein R² is H.

In another embodiment, the TOR kinase inhibitors of formula (Ia) are those wherein Y is CH.

In another embodiment, the TOR kinase inhibitors of formula (Ia) are those wherein L is a direct bond.

In another embodiment, the TOR kinase inhibitors of formula (Ia) are those wherein R¹ is substituted or unsubstituted aryl and R² is unsubstituted C₁₋₈alkyl.

In another embodiment, the TOR kinase inhibitors of formula (Ia) are those wherein R¹ is substituted or unsubstituted aryl and R² is C₁₋₈alkyl substituted with one or more substituents selected from alkoxy, amino, hydroxy, cycloalkyl, or heterocyclylalkyl.

In another embodiment, the TOR kinase inhibitors of formula (Ia) are those wherein R¹ is substituted or unsubstituted aryl and R² is substituted or unsubstituted cycloalkyl, or substituted or unsubstituted heterocyclylalkyl.

In another embodiment, the TOR kinase inhibitors of formula (Ia) do not include compounds wherein Y is CH, L is a direct bond, R¹ is substituted or unsubstituted aryl or substituted or unsubstituted heteroaryl, and R² is C₁₋₈alkyl substituted with substituted or unsubstituted aryl or substituted or unsubstituted heteroaryl.

In one embodiment, the TOR kinase inhibitors include compounds having the following formula (Ib):

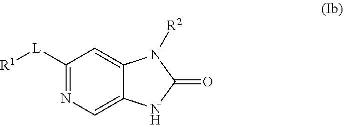

and pharmaceutically acceptable salts, clathrates, solvates, stereoisomers, tautomers, and prodrugs thereof, wherein:

L is a direct bond, NH or O;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl; and

R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (Ib) are those wherein R¹ is substituted aryl, such as substituted phenyl.

In another embodiment, the TOR kinase inhibitors of formula (Ib) are those wherein R¹ is substituted or unsubstituted aryl, such as substituted or unsubstituted phenyl or substituted or unsubstituted naphthyl.

In another embodiment, the TOR kinase inhibitors of formula (Ib) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (Ib) are those wherein R¹ is H.

In another embodiment, the TOR kinase inhibitors of formula (Ib) are those wherein R² is substituted C₁₋₈alkyl.

In another embodiment, the TOR kinase inhibitors of formula (Ib) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (Ib) are those wherein R² is substituted or unsubstituted cycloalkyl or substituted or unsubstituted heterocyclylalkyl.

In another embodiment, the TOR kinase inhibitors of formula (Ib) are those wherein R² is substituted or unsubstituted aryl, such as substituted or unsubstituted phenyl.

In another embodiment, the TOR kinase inhibitors of formula (Ib) are those wherein R² is H.

In another embodiment, the TOR kinase inhibitors of formula (Ib) are those wherein L is a direct bond.

In another embodiment, the TOR kinase inhibitors of formula (Ib) are those wherein R¹ is substituted or unsubstituted aryl and R² is unsubstituted C₁₋₈alkyl.

In another embodiment, the TOR kinase inhibitors of formula (Ib) are those wherein R¹ is substituted or unsubstituted aryl and R² is C₁₋₈alkyl substituted with one or more substituents selected from alkoxy, amino, hydroxy, cycloalkyl, or heterocyclylalkyl.

In another embodiment, the TOR kinase inhibitors of formula (Ib) are those wherein R¹ is substituted or unsubstituted aryl and R² is substituted or unsubstituted cycloalkyl, or substituted or unsubstituted heterocyclylalkyl.

In one embodiment, the TOR kinase inhibitors include compounds having the following formula (Ic):

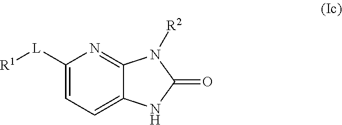

and pharmaceutically acceptable salts, clathrates, solvates, stereoisomers, tautomers, and prodrugs thereof, wherein:

L is a direct bond, NH or O;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl; and

R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (Ic) are those wherein R¹ is substituted aryl, such as substituted phenyl.

In another embodiment, the TOR kinase inhibitors of formula (Ic) are those wherein R¹ is substituted or unsubstituted aryl, such as substituted or unsubstituted phenyl or substituted or unsubstituted naphthyl.

In another embodiment, the TOR kinase inhibitors of formula (Ic) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (Ic) are those wherein R¹ is H.

In another embodiment, the TOR kinase inhibitors of formula (Ic) are those wherein R² is substituted C₁₋₈alkyl.

In another embodiment, the TOR kinase inhibitors of formula (Ic) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (Ic) are those wherein R² is substituted or unsubstituted cycloalkyl or substituted or unsubstituted heterocyclylalkyl.

In another embodiment, the TOR kinase inhibitors of formula (Ic) are those wherein R² is substituted or unsubstituted aryl, such as substituted or unsubstituted phenyl.

In another embodiment, the TOR kinase inhibitors of formula (Ic) are those wherein R² is H.

In another embodiment, the TOR kinase inhibitors of formula (Ic) are those wherein L is a direct bond.

In another embodiment, the TOR kinase inhibitors of formula (Ic) are those wherein R¹ is substituted or unsubstituted aryl and R² is unsubstituted C₁₋₈alkyl.

In another embodiment, the TOR kinase inhibitors of formula (Ic) are those wherein R¹ is substituted or unsubstituted aryl and R² is C₁₋₈alkyl substituted with one or more substituents selected from alkoxy, amino, hydroxy, cycloalkyl, or heterocyclylalkyl.

In another embodiment, the TOR kinase inhibitors of formula (Ic) are those wherein R¹ is substituted or unsubstituted aryl and R² is substituted or unsubstituted cycloalkyl, or substituted or unsubstituted heterocyclylalkyl.

In one embodiment, the TOR kinase inhibitors include compounds having the following formula (Id):

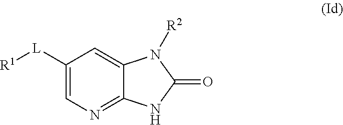

and pharmaceutically acceptable salts, clathrates, solvates, stereoisomers, tautomers, and prodrugs thereof, wherein:

L is a direct bond, NH or O;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl; and

R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (Id) are those wherein R¹ is substituted aryl, such as substituted phenyl.

In another embodiment, the TOR kinase inhibitors of formula (Id) are those wherein R¹ is substituted or unsubstituted aryl, such as substituted or unsubstituted phenyl or substituted or unsubstituted naphthyl.

In another embodiment, the TOR kinase inhibitors of formula (Id) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (Id) are those wherein R¹ is H.

In another embodiment, the TOR kinase inhibitors of formula (Id) are those wherein R² is substituted C₁₋₈alkyl.

In another embodiment, the TOR kinase inhibitors of formula (Id) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (Id) are those wherein R² is substituted or unsubstituted cycloalkyl or substituted or unsubstituted heterocyclylalkyl.

In another embodiment, the TOR kinase inhibitors of formula (Id) are those wherein R² is substituted or unsubstituted aryl, such as substituted or unsubstituted phenyl.

In another embodiment, the Heteroaryl Compounds of formula (Id) are those wherein R² is H.

In another embodiment, the TOR kinase inhibitors of formula (Id) are those wherein L is a direct bond.

In another embodiment, the TOR kinase inhibitors of formula (Id) are those wherein R¹ is substituted or unsubstituted aryl and R² is unsubstituted C₁₋₈alkyl.

In another embodiment, the TOR kinase inhibitors of formula (Id) are those wherein R¹ is substituted or unsubstituted aryl and R² is C₁₋₈alkyl substituted with one or more substituents selected from alkoxy, amino, hydroxy, cycloalkyl, or heterocyclylalkyl.

In another embodiment, the TOR kinase inhibitors of formula (Id) are those wherein R¹ is substituted or unsubstituted aryl and R² is substituted or unsubstituted cycloalkyl, or substituted or unsubstituted heterocyclylalkyl.

In one embodiment, the TOR kinase inhibitors include compounds having the following formula (Ie):

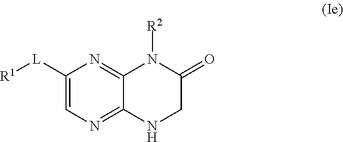

and pharmaceutically acceptable salts, clathrates, solvates, stereoisomers, tautomers, and prodrugs thereof, wherein:

L is a direct bond, NH or O;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl; and R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (Ie) are those wherein R¹ is substituted aryl, such as substituted phenyl.

In another embodiment, the TOR kinase inhibitors of formula (Ie) are those wherein R¹ is substituted or unsubstituted aryl, such as substituted or unsubstituted phenyl or substituted or unsubstituted naphthyl.

In another embodiment, the TOR kinase inhibitors of formula (Ie) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (Ie) are those wherein R¹ is H.

In another embodiment, the TOR kinase inhibitors of formula (Ie) are those wherein R² is substituted C₁₋₈alkyl.

In another embodiment, the TOR kinase inhibitors of formula (Ie) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (Ie) are those wherein R² is substituted or unsubstituted cycloalkyl or substituted or unsubstituted heterocyclylalkyl.

In another embodiment, the TOR kinase inhibitors of formula (Ie) are those wherein R² is substituted or unsubstituted aryl, such as substituted or unsubstituted phenyl.

In another embodiment, the TOR kinase inhibitors of formula (Ie) are those wherein R² is H.

In another embodiment, the TOR kinase inhibitors of formula (Ie) are those wherein L is a direct bond.

In another embodiment, the TOR kinase inhibitors of formula (Ie) are those wherein R¹ is substituted or unsubstituted aryl and R² is unsubstituted C₁₋₈alkyl.

In another embodiment, the TOR kinase inhibitors of formula (Ie) are those wherein R¹ is substituted or unsubstituted aryl and R² is C₁₋₈alkyl substituted with one or more substituents selected from alkoxy, amino, hydroxy, cycloalkyl, or heterocyclylalkyl.

In another embodiment, the TOR kinase inhibitors of formula (Ie) are those wherein R¹ is substituted or unsubstituted aryl and R² is substituted or unsubstituted cycloalkyl, or substituted or unsubstituted heterocyclylalkyl.

In one embodiment, the TOR kinase inhibitors include compounds having the following formula (If):

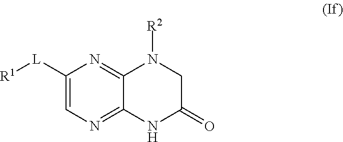

and pharmaceutically acceptable salts, clathrates, solvates, stereoisomers, tautomers, and prodrugs thereof, wherein:

L is a direct bond, NH or O;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl; and

R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (If) are those wherein R¹ is substituted aryl, such as substituted phenyl.

In another embodiment, the TOR kinase inhibitors of formula (If) are those wherein R¹ is substituted or unsubstituted aryl, such as substituted or unsubstituted phenyl or substituted or unsubstituted naphthyl.

In another embodiment, the TOR kinase inhibitors of formula (If) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (If) are those wherein R¹ is H.

In another embodiment, the TOR kinase inhibitors of formula (If) are those wherein R² is substituted C₁₋₈alkyl.

In another embodiment, the TOR kinase inhibitors of formula (If) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (If) are those wherein R² is substituted or unsubstituted cycloalkyl or substituted or unsubstituted heterocyclylalkyl.

In another embodiment, the TOR kinase inhibitors of formula (If) are those wherein R² is substituted or unsubstituted aryl, such as substituted or unsubstituted phenyl.

In another embodiment, the TOR kinase inhibitors of formula (If) are those wherein R² is H.

In another embodiment, the TOR kinase inhibitors of formula (If) are those wherein L is a direct bond.

In another embodiment, the TOR kinase inhibitors of formula (If) are those wherein R¹ is substituted or unsubstituted aryl and R² is unsubstituted C₁₋₈alkyl.

In another embodiment, the TOR kinase inhibitors of formula (If) are those wherein R¹ is substituted or unsubstituted aryl and R² is C₁₋₈alkyl substituted with one or more substituents selected from alkoxy, amino, hydroxy, cycloalkyl, or heterocyclylalkyl.

In another embodiment, the TOR kinase inhibitors of formula (If) are those wherein R¹ is substituted or unsubstituted aryl and R² is substituted or unsubstituted cycloalkyl, or substituted or unsubstituted heterocyclylalkyl.

In one embodiment, the TOR kinase inhibitors include compounds having the following formula (Ig):

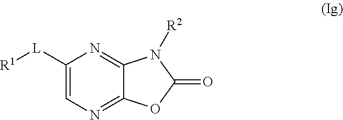

and pharmaceutically acceptable salts, clathrates, solvates, stereoisomers, tautomers, and prodrugs thereof, wherein:

L is a direct bond, NH or O;

R¹ is H, substituted or unsubstituted C₁₋₈alkyl, substituted or unsubstituted C₂₋₈alkenyl, substituted or unsubstituted aryl, substituted or unsubstituted heteroaryl, substituted or unsubstituted cycloalkyl or substituted or unsubstituted heterocyclylalkyl; and

R² is H, substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl.

In one embodiment, the TOR kinase inhibitors of formula (Ig) are those wherein R¹ is substituted aryl, such as substituted phenyl.

In another embodiment, the TOR kinase inhibitors of formula (Ig) are those wherein R¹ is substituted or unsubstituted aryl, such as substituted or unsubstituted phenyl or substituted or unsubstituted naphthyl.

In another embodiment, the TOR kinase inhibitors of formula (Ig) are those wherein R¹ is substituted or unsubstituted heteroaryl, such as substituted or unsubstituted quinoline, substituted or unsubstituted pyridine, substituted or unsubstituted pyrimidine, substituted or unsubstituted indole, or substituted or unsubstituted thiophene.

In another embodiment, the TOR kinase inhibitors of formula (Ig) are those wherein R¹ is H.

In another embodiment, the TOR kinase inhibitors of formula (Ig) are those wherein R² is substituted C₁₋₈alkyl.

In another embodiment, the TOR kinase inhibitors of formula (Ig) are those wherein R² is methyl or ethyl substituted with substituted or unsubstituted aryl, substituted or unsubstituted heteroaryl, substituted or unsubstituted cycloalkyl, or substituted or unsubstituted heterocyclylalkyl.

In another embodiment, the TOR kinase inhibitors of formula (Ig) are those wherein R² is substituted or unsubstituted cycloalkyl or substituted or unsubstituted heterocyclylalkyl.

In another embodiment, the TOR kinase inhibitors of formula (Ig) are those wherein R² is substituted or unsubstituted aryl, such as substituted or unsubstituted phenyl.

In another embodiment, the TOR kinase inhibitors of formula (Ig) are those wherein R² is H.

In another embodiment, the TOR kinase inhibitors of formula (Ig) are those wherein L is a direct bond.

In another embodiment, the TOR kinase inhibitors of formula (Ig) are those wherein R¹ is substituted or unsubstituted aryl and R² is unsubstituted C₁₋₈alkyl.

In another embodiment, the TOR kinase inhibitors of formula (Ig) are those wherein R¹ is substituted or unsubstituted aryl and R² is C₁₋₈alkyl substituted with one or more substituents selected from alkoxy, amino, hydroxy, cycloalkyl, or heterocyclylalkyl.

In another embodiment, the TOR kinase inhibitors of formula (Ig) are those wherein R¹ is substituted or unsubstituted aryl and R² is substituted or unsubstituted cycloalkyl, or substituted or unsubstituted heterocyclylalkyl.

Representative TOR kinase inhibitors of formula (I) include:
(S)-1-(1-hydroxy-3-methylbutan-2-yl)-6-phenyl-1H-imidazo[4,5-b]pyrazin-2(3H)-one;
1-((tetrahydro-2H-pyran-4-yl)methyl)-6-(3,4,5-trimethoxyphenyl)-1H-imidazo[4,5-b]pyrazin-2(3H)-one;
(R)-6-(naphthalen-1-yl)-1-(1-phenylethyl)-1H-imidazo[4,5-b]pyrazin-2(3H)-one;
1-(3-methoxybenzyl)-6-(4-(methylsulfonyl)phenyl)-1H-imidazo[4,5-b]pyrazin-2(3H)-one;
(S)-1-(1-phenylethyl)-6-(quinlin-5-yl)-1H-imidazo[4,5-b]pyrazin-2(3H)-one;
6-(4-hydroxyphenyl)-1-((tetrahydro-2H-pyran-4-yl)methyl)-1H-imidazo[4,5-b]pyrazin-2(3H)-one;
(S)-6-(naphthalen-1-yl)-1-(1-phenylethyl)-1H-imidazo[4,5-b]pyrazin-2(3H)-one;
(S)-1-(1-hydroxy-3-methylbutan-2-yl)-6-(5-isopropyl-2-methoxyphenyl)-1H-imidazo[4,5-b]pyrazin-2(3H)-one;
(R)-1-(1-hydroxy-3-methylbutan-2-yl)-6-phenyl-1H-imidazo[4,5-b]pyrazin-2(3H)-one;
(R)-1-(1-phenylethyl)-6-(quinolin-5-yl)-1H-imidazo[4,5-b]pyrazin-2(3H)-one;
(S)-1-(1-hydroxy-3-methylbutan-2-yl)-6-(quinolin-5-yl)-1H-imidazo[4,5-b]pyrazin-2(3H)-one;
(R)-1-(1-hydroxy-3-methylbutan-2-yl)-6-(quinolin-5-yl)-1H-imidazo[4,5-b]pyrazin-2(3H)-one;
(R)-1-(1-hydroxy-3-methylbutan-2-yl)-6-(5-isopropyl-2-methoxyphenyl)-1H-imidazo[4,5-b]pyrazin-2(3H)-one;
1-benzyl-6-(quinolin-5-yl)-1H-imidazo[4,5-b]pyrazin-2(3H)-one;
1-(4-methoxybenzyl)-6-(quinolin-5-yl)-1H-imidazo[4,5-b]pyrazin-2(3H)-one;
(R)-1-(1-phenylethyl)-1H-imidazo[4,5-b]pyrazin-2(3H)-one;
(S)-1-(1-phenylethyl)-1H-imidazo[4,5-b]pyrazin-2(3H)-one;
1-isopropyl-6-(5-isopropyl-2-methoxyphenyl)-1H-imidazo[4,5-b]pyrazin-2(3H)-one;
1-cyclohexyl-6-(5-isopropyl-2-methoxyphenyl)-1H-imidazo[4,5-b]pyrazin-2(3H)-one;
5-(quinolin-5-yl)-1H-imidazo[4,5-b]pyrazin-2(3H)-one;
1-isobutyl-6-(5-isopropyl-2-methoxyphenyl)-1H-imidazo[4,5-b]pyrazin-2(3H)-one;
1-(2-hydroxyethyl)-6-(5-isopropyl-2-methoxyphenyl)-1H-imidazo[4,5-b]pyrazin-2(3H)-one;
6-(5-isopropyl-2-methoxyphenyl)-1-(tetrahydro-2H-pyran-4-yl)-1H-imidazo[4,5-b]pyrazin-2(3H)-one;
(R)-1-(1-phenylethyl)-6-(quinolin-5-yl)-1H-imidazo[4,5-c]pyridin-2(3H)-one;
(S)-1-(1-phenylethyl)-6-(quinolin-5-yl)-1H-imidazo[4,5-c]pyridin-2(3H)-one;
3-(1-phenylethyl)-5-(quinolin-5-yl)-1H-imidazo[4,5-b]pyridin-2(3H)-one;
(R)-3-(1-phenylethyl)-5-(quinolin-5-yl)-1H-imidazo[4,5-b]pyridin-2(3H)-one;
(R)-6-(5-isopropyl-2-methoxyphenyl)-1-(3-methylbutan-2-yl)-1H-imidazo[4,5-b]pyrazin-2(3H)-one;
(S)-6-(5-isopropyl-2-methoxyphenyl)-1-(tetrahydrofuran-3-yl)-1H-imidazo[4,5-b]pyrazin-2(3H)-one;
(S)-6-(5-isopropyl-2-methoxyphenyl)-1-(3-methylbutan-2-yl)-1H-imidazo[4,5-b]pyrazin-2(3H)-one;
1-cyclopentyl-6-(5-isopropyl-2-methoxyphenyl)-1H-imidazo[4,5-b]pyrazin-2(3H)-one;
(R)-6-(5-isopropyl-2-methoxyphenyl)-1-(tetrahydrofuran-3-yl)-1H-imidazo[4,5-b]pyrazin-2(3H)-one;
1-(cyclopropylmethyl)-6-(5-isopropyl-2-methoxyphenyl)-1H-imidazo[4,5-b]pyrazin-2(3H)-one;
1-(cyclopentylmethyl)-6-(5-isopropyl-2-methoxyphenyl)-1H-imidazo[4,5-b]pyrazin-2(3H)-one;
1-(cyclohexylmethyl)-6-(5-isopropyl-2-methoxyphenyl)-1H-imidazo[4,5-b]pyrazin-2(3H)-one;
6-(5-isopropyl-2-methoxyphenyl)-1-neopentyl-1H-imidazo[4,5-b]pyrazin-2(3H)-one;
1-isopropyl-6-(3-isopropylphenyl)-1H-imidazo[4,5-b]pyrazin-2(3H)-one;
1-isopropyl-6-(2-methoxyphenyl)-1H-imidazo[4,5-b]pyrazin-2(3H)-one;
(S)-3-(1-hydroxy-3-methylbutan-2-yl)-5-(5-isopropyl-2-methoxyphenyl)-1H-imidazo[4,5-b]pyridin-2(3H)-one;
(R)-1-(2-hydroxy-1-phenylethyl)-6-(quinolin-5-yl)-1H-imidazo[4,5-b]pyrazin-2(3H)-one;
(S)-1-(2-hydroxy-1-phenylethyl)-6-(quinolin-5-yl)-1H-imidazo[4,5-b]pyrazin-2(3H)-one;
1-(1-phenylethyl)-6-(quinolin-5-yl)-1H-imidazo[4,5-b]pyrazin-2(3H)-one;
1-benzhydryl-6-(quinolin-5-yl)-1H-imidazo[4,5-b]pyrazin-2(3H)-one;
(S)-1-(1-phenylpropyl)-6-(quinolin-5-yl)-1H-imidazo[4,5-b]pyrazin-2(3H)-one;
(R)-1-(1-phenylpropyl)-6-(quinolin-5-yl)-1H-imidazo[4,5-b]pyrazin-2(3H)-one;
6-(5-isopropyl-2-methoxyphenyl)-1-(tetrahydro-2H-pyran-3-yl)-1H-imidazo[4,5-b]pyrazin-2(3H)-one;
1-(3-methoxybenzyl)-6-(quinolin-5-yl)-1H-imidazo[4,5-b]pyrazin-2(3H)-one;
(R)-1-methyl-3-(1-phenylethyl)-5-(quinolin-5-yl)-1H-imidazo[4,5-b]pyrazin-2(3H)-one;
(S)-1-methyl-3-(1-phenylethyl)-5-(quinolin-5-yl)-1H-imidazo[4,5-b]pyrazin-2(3H)-one;
1-(cyclopentylmethyl)-6-(quinolin-5-yl)-1H-imidazo[4,5-b]pyrazin-2(3H)-one;
1-(1-(2-fluorophenyl)ethyl)-6-(quinolin-5-yl)-1H-imidazo[4,5-b]pyrazin-2(3H)-one;
1-(1-(4-fluorophenyl)ethyl)-6-(quinolin-5-yl)-1H-imidazo[4,5-b]pyrazin-2(3H)-one;
1-cyclopentyl-6-(quinolin-5-yl)-1H-imidazo[4,5-b]pyrazin-2(3H)-one;
1-(1-(3-fluorophenyl)ethyl)-6-(quinolin-5-yl)-1H-imidazo[4,5-b]pyrazin-2(3H)-one;
1-(1-(3-methoxyphenyl)ethyl)-6-(quinolin-5-yl)-1H-imidazo[4,5-b]pyrazin-2(3H)-one;
1-(1-(4-methoxyphenyl)ethyl)-6-(quinolin-5-yl)-1H-imidazo[4,5-b]pyrazin-2(3H)-one;
6-(quinolin-5-yl)-1-(tetrahydro-2H-pyran-4-yl)-1H-imidazo[4,5-b]pyrazin-2(3H)-one;
6-(quinolin-5-yl)-1-(tetrahydro-2H-pyran-3-yl)-1H-imidazo[4,5-b]pyrazin-2(3H)-one;
1-((1s,4s)-4-hydroxycyclohexyl)-6-(quinolin-5-yl)-1H-imidazo[4,5-b]pyrazin-2(3H)-one;
1-((1r,4r)-4-hydroxycyclohexyl)-6-(quinolin-5-yl)-1H-imidazo[4,5-b]pyrazin-2(3H)-one;
6-(isoquinolin-5-yl)-1-(1-phenylethyl)-1H-imidazo[4,5-b]pyrazin-2(3H)-one;
(R)-1-(1-phenylethyl)-6-(quinolin-5-yl)-1H-imidazo[4,5-b]pyridin-2(3H)-one;
1-(1-phenylethyl)-6-(quinolin-5-yl)-1H-imidazo[4,5-b]pyridin-2(3H)-one;
1-isopropyl-6-(quinolin-5-yl)-1H-imidazo[4,5-b]pyrazin-2(3H)-one;
1-(1-(4-chlorophenyl)ethyl)-6-(quinolin-5-yl)-1H-imidazo[4,5-b]pyrazin-2(3H)-one;
1-(1-(4-(methylsulfonyl)phenyl)ethyl)-6-(quinolin-5-yl)-1H-imidazo[4,5-b]pyrazin-2(3H)-one;
1-(1-(pyridin-4-yl)ethyl)-6-(quinolin-5-yl)-1H-imidazo[4,5-b]pyrazin-2(3H)-one;
5-methyl-1-((S)-1-phenylethyl)-6-(quinolin-5-yl)-1H-imidazo[4,5-b]pyrazin-2(3H)-one;
5-methyl-1-((R)-1-phenylethyl)-6-(quinolin-5-yl)-1H-imidazo[4,5-b]pyrazin-2(3H)-one;
1-(1-phenylethyl)-6-(quinolin-4-yl)-1H-imidazo[4,5-b]pyrazin-2(3H)-one;
6-(3-fluorophenyl)-1-(1-phenylethyl)-1H-imidazo[4,5-b]pyrazin-2(3H)-one;
6-(2-fluorophenyl)-1-(1-phenylethyl)-1H-imidazo[4,5-b]pyrazin-2(3H)-one;
1-(1-phenylethyl)-6-(quinolin-6-yl)-1H-imidazo[4,5-b]pyrazin-2(3H)-one;
1-(piperidin-4-ylmethyl)-6-(quinolin-5-yl)-1H-imidazo[4,5-b]pyrazin-2(3H)-one;
1-(1-(pyridin-2-yl)ethyl)-6-(quinolin-5-yl)-1H-imidazo[4,5-b]pyrazin-2(3H)-one;
1-(1-(pyridin-3-yl)ethyl)-6-(quinolin-5-yl)-1H-imidazo[4,5-b]pyrazin-2(3H)-one;
1-((1s,4s)-4-(hydroxymethyl)cyclohexyl)-6-(quinolin-5-yl)-1H-imidazo[4,5-b]pyrazin-2(3H)-one;
N-(4-(2-oxo-3-(1-phenylethyl)-2,3-dihydro-1H-imidazo[4,5-b]pyrazin-5-yl)phenyl)methanesulfonamide;
6-(3-(methylsulfonyl)phenyl)-1-(1-phenylethyl)-1H-imidazo[4,5-b]pyrazin-2(3H)-one;
6-(3-aminophenyl)-1-(1-phenylethyl)-1H-imidazo[4,5-b]pyrazin-2(3H)-one;
6-(3-(dimethylamino)phenyl)-1-(1-phenylethyl)-1H-imidazo[4,5-b]pyrazin-2(3H)-one;
1-phenyl-6-(quinolin-5-yl)-1H-imidazo[4,5-b]pyrazin-2(3H)-one;
1-(1-phenylethyl)-6-(4-(trifluoromethyl)phenyl)-1H-imidazo[4,5-b]pyrazin-2(3H)-one;
N-(3-(2-oxo-3-(1-phenylethyl)-2,3-dihydro-1H-imidazo[4,5-b]pyrazin-5-yl)phenyl)methanesulfonamide;
6-(4-(methylsulfonyl)phenyl)-1-(1-phenylethyl)-1H-imidazo[4,5-b]pyrazin-2(3H)-one;
3-(1-phenylethyl)-5-(quinolin-5-yl)oxazolo[5,4-b]pyrazin-2(3H)-one;
1-(cyclopentylmethyl)-6-(4-hydroxyphenyl)-1H-imidazo[4,5-b]pyrazin-2(3H)-one
6-(4-hydroxyphenyl)-1-isopropyl-1H-imidazo[4,5-b]pyrazin-2(3H)-one;
6-(4-hydroxyphenyl)-1-isobutyl-1H-imidazo[4,5-b]pyrazin-2(3H)-one;
6-(4-hydroxyphenyl)-1-((tetrahydro-2H-pyran-3-yl)methyl)-1H-imidazo[4,5-b]pyrazin-2(3H)-one;
1-(cyclohexylmethyl)-6-(4-hydroxyphenyl)-1H-imidazo[4,5-b]pyrazin-2(3H)-one;
5-(3-Hydroxyphenyl)-3-(2-methoxyphenyl)-1H-imidazo[4,5-b]pyridin-2(3H)-one;
4-(3-(3-Methoxybenzyl)-2-oxo-2,3-dihydrooxazolo[5,4-b]pyrazin-5-yl)-N-methyl benzamide;
1-Cyclopentyl-6-(4-hydroxyphenyl)-1H-imidazo[4,5-b]pyrazin-2(3H)-one;
1-Cyclohexyl-6-(4-hydroxyphenyl)-1H-imidazo[4,5-b]pyrazin-2(3H)-one;
4-(3-(Cyclohexylmethyl)-2-oxo-2,3-dihydro-1H-imidazo[4,5-b]pyrazin-5-yl)benzamide;
Methyl 4-(3-(cyclohexylmethyl)-2-oxo-2,3-dihydro-1H-imidazo[4,5-b]pyrazin-5-yl)benzoate;
1-(Cyclohexylmethyl)-6-(pyridin-4-yl)-1H-imidazo[4,5-b]pyrazin-2(3H)-one;
4-(3-(Cyclohexylmethyl)-2-oxo-2,3-dihydro-1H-imidazo[4,5-b]pyrazin-5-yl)-N-methylbenzamide;
1-(Cyclohexylmethyl)-6-(4-(hydroxymethyl)phenyl)-1H-imidazo[4,5-b]pyrazin-2(3H)-one;
1-(Cyclohexylmethyl)-6-(pyridin-3-yl)-1H-imidazo[4,5-b]pyrazin-2(3H)-one;
3-(Cyclohexylmethyl)-2-oxo-2,3-dihydro-1H-imidazo[4,5-b]pyrazin-5-yl)benzonitrile;
1-(Cyclohexylmethyl)-6-(1H-indol-5-yl)-1H-imidazo[4,5-b]pyrazin-2(3H)-one;
4-(3-(Cyclohexylmethyl)-2-oxo-2,3-dihydro-1H-imidazo[4,5-b]pyrazin-5-yl)-N-isopropylbenz amide;
1-(2-Hydroxyethyl)-6-(4-hydroxyphenyl)-1H-imidazo[4,5-b]pyrazin-2(3H)-one;
1-(Cyclohexylmethyl)-6-(1H-indol-6-yl)-1H-imidazo[4,5-b]pyrazin-2(3H)-one;
3-(3-(Cyclohexylmethyl)-2-oxo-2,3-dihydro-1H-imidazo[4,5-b]pyrazin-5-yl)benzamide;
6-(4-(Aminomethyl)phenyl)-1-(cyclohexylmethyl)-1H-imidazo[4,5-b]pyrazin-2(3H)-one;
6-(4-Hydroxyphenyl)-1-((1-methylpiperidin-4-yl)methyl)-1H-imidazo[4,5-b]pyrazin-2(3H)-one;
4-(3-(Cyclohexylmethyl)-2-oxo-2,3-dihydro-1H-imidazo[4,5-b]pyrazin-5-yl)benzonitrile;
1-((1s,4s)-4-Hydroxycyclohexyl)-6-(4-hydroxyphenyl)-1H-imidazo[4,5-b]pyrazin-2(3H)-one;
1-(Cyclohexylmethyl)-6-(pyridin-2-yl)-1H-imidazo[4,5-b]pyrazin-2(3H)-one;
4-(3-(Cyclohexylmethyl)-2-oxo-2,3-dihydro-1H-imidazo[4,5-b]pyrazin-5-yl)-N-ethylbenzamide;
1-(Cyclohexylmethyl)-6-(4-(2-hydroxypropan-2-yl)phenyl)-1H-imidazo[4,5-b]pyrazin-2(3H)-one;
1-(Cyclohexylmethyl)-6-(4-hydroxy-2-methylphenyl)-1H-imidazo[4,5-b]pyrazin-2(3H)-one;
4-(3-(Cyclohexylmethyl)-2-oxo-2,3-dihydro-1H-imidazo[4,5-b]pyrazin-5-yl)benzoic acid;
6-(4-Hydroxyphenyl)-1-(2-methoxyethyl)-1H-imidazo[4,5-b]pyrazin-2(3H)-one;
6-(4-Hydroxyphenyl)-1-(3-methoxypropyl)-1H-imidazo[4,5-b]pyrazin-2(3H)-one;
6-(4-Hydroxyphenyl)-4-(3-methoxybenzyl)-3,4-dihydropyrazino[2,3-b]pyrazin-2(1H)-one;
6-(4-Hydroxyphenyl)-1-(2-(tetrahydro-2H-pyran-4-yl)ethyl)-1H-imidazo[4,5-b]pyrazin-2(3H)-one;
6-(4-Hydroxyphenyl)-1-phenethyl-1H-imidazo[4,5-b]pyrazin-2(3H)-one;
1-((1r,4r)-4-Hydroxycyclohexyl)-6-(4-hydroxyphenyl)-1H-imidazo[4,5-b]pyrazin-2(3H)-one;
6-(4-(1H-1,2,4-Triazol-3-yl)phenyl)-1-(cyclohexylmethyl)-1H-imidazo[4,5-b]pyrazin-2(3H)-one;
1-(Cyclohexylmethyl)-6-phenyl-1H-imidazo[4,5-b]pyrazin-2(3H)-one;
1-(Cyclohexylmethyl)-6-(1H-pyrazol-5-yl)-1H-imidazo[4,5-b]pyrazin-2(3H)-one;
1-(Cyclohexylmethyl)-6-(1H-pyrazol-4-yl)-1H-imidazo[4,5-b]pyrazin-2(3H)-one;
1-(Cyclohexylmethyl)-6-(1-oxoisoindolin-5-yl)-1H-imidazo[4,5-b]pyrazin-2(3H)-one;
6-(3-(1H-Tetrazol-5-yl)phenyl)-1-(cyclohexylmethyl)-1H-imidazo[4,5-b]pyrazin-2(3H)-one;
1-(Cyclohexylmethyl)-6-(2-oxoindolin-5-yl)-1H-imidazo[4,5-b]pyrazin-2(3H)-one;
1-(Cyclohexylmethyl)-6-(1H-indazol-5-yl)-1H-imidazo[4,5-b]pyrazin-2(3H)-one;
1-(Cyclohexylmethyl)-6-(6-methoxypyridin-3-yl)-1H-imidazo[4,5-b]pyrazin-2(3H)-one;
6-(4-Hydroxyphenyl)-1-(tetrahydro-2H-pyran-4-yl)-1H-imidazo[4,5-b]pyrazin-2(3H)-one;
6-(4-Hydroxyphenyl)-1-(piperidin-4-ylmethyl)-1H-imidazo[4,5-b]pyrazin-2(3H)-one;
1-(((1r,4r)-4-Aminocyclohexyl)methyl)-6-(4-hydroxyphenyl)-1H-imidazo[4,5-b]pyrazin-2(3H)-one;
1-(Cyclohexylmethyl)-6-(6-hydroxypyridin-3-yl)-1H-imidazo[4,5-b]pyrazin-2(3H)-one;
1-(Cyclohexylmethyl)-6-(2-methoxypyridin-4-yl)-1H-imidazo[4,5-b]pyrazin-2(3H)-one;
4-(3-((1r,4r)-4-Hydroxycyclohexyl)-2-oxo-2,3-dihydro-1H-imidazo[4,5-b]pyrazin-5-yl)benzamide;
2-(4-(3-(Cyclohexylmethyl)-2-oxo-2,3-dihydro-1H-imidazo[4,5-b]pyrazin-5-yl)phenyl)acetic acid;
2-(4-(3-(Cyclohexylmethyl)-2-oxo-2,3-dihydro-1H-imidazo[4,5-b]pyrazin-5-yl)phenyl) acetamide;
1-(Cyclohexylmethyl)-6-(2-oxoindolin-6-yl)-1H-imidazo[4,5-b]pyrazin-2(3H)-one;
4-(3-(Cyclohexylmethyl)-2-oxo-2,3-dihydro-1H-imidazo[4,5-b]pyrazin-5-yl)-3-methyl benzoic acid;
N-Methyl-4-(2-oxo-3-((tetrahydro-2H-pyran-4-yl)methyl)-2,3-dihydro-1H-imidazo[4,5-b]pyrazin-5-yl)benzamide;
4-(2-oxo-3-((Tetrahydro-2H-pyran-4-yl)methyl)-2,3-dihydro-1H-imidazo[4,5-b]pyrazin-5-yl)benzamide;
7-(4-Hydroxyphenyl)-1-(3-methoxybenzyl)-3,4-dihydropyrazino[2,3-b]pyrazin-2(1H)-one;
6-(4-(2-Hydroxypropan-2-yl)phenyl)-1-((tetrahydro-2H-pyran-4-yl)methyl)-1H-imidazo[4,5-b]pyrazin-2(3H)-one;
6-(1H-Indol-5-yl)-1-((tetrahydro-2H-pyran-4-yl)methyl)-1H-imidazo[4,5-b]pyrazin-2(3H)-one;
6-(4-(4H-1,2,4-Triazol-3-yl)phenyl)-1-((tetrahydro-2H-pyran-4-yl)methyl)-1H-imidazo[4,5-b]pyrazin-2(3H)-one;
6-(1H-Benzo[d]imidazol-5-yl)-1-(cyclohexylmethyl)-1H-imidazo[4,5-b]pyrazin-2(3H)-one;
4-(2-oxo-3-(2-(Tetrahydro-2H-pyran-4-yl)ethyl)-2,3-dihydro-1H-imidazo[4,5-b]pyrazin-5-yl)benzamide;
6-(3-(2H-1,2,3-Triazol-4-yl)phenyl)-1-(cyclohexylmethyl)-1H-imidazo[4,5-b]pyrazin-2(3H)-one;
6-(4-(1H-Imidazol-1-yl)phenyl)-1-(cyclohexylmethyl)-1H-imidazo[4,5-b]pyrazin-2(3H)-one;
6-(4-(1H-1,2,4-Triazol-3-yl)phenyl)-1-((1r,4r)-4-hydroxycyclohexyl)-1H-imidazo[4,5-b]pyrazin-2(3H)-one;
6-(4-(2H-tetrazol-5-yl)phenyl)-1-(cyclohexylmethyl)-1H-imidazo[4,5-b]pyrazin-2(3H)-one;
1-(Cyclohexylmethyl)-6-(2-hydroxypyridin-4-yl)-1H-imidazo[4,5-b]pyrazin-2(3H)-one;
6-(4-(1H-1,2,4-Triazol-3-yl)phenyl)-1-(2-(tetrahydro-2H-pyran-4-yl)ethyl)-1H-imidazo[4,5-b]pyrazin-2(3H)-one;
6-(4-(1H-Imidazol-2-yl)phenyl)-1-(cyclohexylmethyl)-1H-imidazo[4,5-b]pyrazin-2(3H)-one;
6-(4-(1H-1,2,3-Triazol-1-yl)phenyl)-1-(cyclohexylmethyl)-1H-imidazo[4,5-b]pyrazin-2(3H)-one;
6-(4-(2-Hydroxypropan-2-yl)phenyl)-1-(2-(tetrahydro-2H-pyran-4-yl)ethyl)-1H-imidazo[4,5-b]pyrazin-2(3H)-one;
1-(Cyclohexylmethyl)-6-(4-(5-methyl-1H-1,2,4-triazol-3-yl)phenyl)-1H-imidazo[4,5-b]pyrazin-2(3H)-one;
6-(4-(1H-Pyrazol-3-yl)phenyl)-1-(cyclohexylmethyl)-1H-imidazo[4,5-b]pyrazin-2(3H)-one;
6-(4-(1H-Pyrazol-4-yl)phenyl)-1-(cyclohexylmethyl)-1H-imidazo[4,5-b]pyrazin-2(3H)-one;
6-(4-(5-(Aminomethyl)-1H-1,2,4-triazol-3-yl)phenyl)-1-(cyclohexylmethyl)-1H-imidazo[4,5-b]pyrazin-2(3H)-one hydrochloride;
1-(Cyclohexylmethyl)-6-(4-(5-(trifluoromethyl)-1H-1,2,4-triazol-3-yl)phenyl)-1H-imidazo[4,5-b]pyrazin-2(3H)-one;
6-(4-Hydroxyphenyl)-1-((1r,4r)-4-methoxycyclohexyl)-1H-imidazo[4,5-b]pyrazin-2(3H)-one;
6-(4-Hydroxyphenyl)-1-((tetrahydrofuran-2-yl)methyl)-1H-imidazo[4,5-b]pyrazin-2(3H)-one;
6-(3-(1H-1,2,4-Triazol-3-yl)phenyl)-1-(cyclohexylmethyl)-1H-imidazo[4,5-b]pyrazin-2(3H)-one;
1-((1r,4r)-4-(Hydroxymethyl)cyclohexyl)-6-(4-hydroxyphenyl)-1H-imidazo[4,5-b]pyrazin-2(3H)-one;
6-(4-Hydroxyphenyl)-1-((1s,4s)-4-methoxycyclohexyl)-1H-imidazo[4,5-b]pyrazin-2(3H)-one;
6-(4-Hydroxyphenyl)-1-((1r,4r)-4-(methoxymethyl)cyclohexyl)-1H-imidazo[4,5-b]pyrazin-2(3H)-one;
6-(1-Methyl-1H-pyrazol-4-yl)-1-((tetrahydro-2H-pyran-4-yl)methyl)-1H-imidazo[4,5-b]pyrazin-2(3H)-one;
1-(((1r,4r)-4-Hydroxycyclohexyl)methyl)-6-(4-hydroxyphenyl)-1H-imidazo[4,5-b]pyrazin-2(3H)-one;
6-(4-Hydroxyphenyl)-1-((tetrahydrofuran-3-yl)methyl)-1H-imidazo[4,5-b]pyrazin-2(3H)-one;
1-(((1s,4s)-4-Hydroxycyclohexyl)methyl)-6-(4-hydroxyphenyl)-1H-imidazo[4,5-b]pyrazin-2(3H)-one;
6-(1H-Benzo[d]imidazol-5-yl)-1-((tetrahydro-2H-pyran-4-yl)methyl)-1H-imidazo[4,5-b]pyrazin-2(3H)-one hydrochloride;
6-(4-(5-(Morpholinomethyl)-1H-1,2,4-triazol-3-yl)phenyl)-1-((tetrahydro-2H-pyran-4-yl)methyl)-1H-imidazo[4,5-b]pyrazin-2(3H)-one;
6-(4-Hydroxyphenyl)-1-(3-(2-oxopyrrolidin-1-yl)propyl)-1H-imidazo[4,5-b]pyrazin-2(3H)-one;
6-(4-Hydroxyphenyl)-1-(2-morpholinoethyl)-1H-imidazo[4,5-b]pyrazin-2(3H)-one hydrochloride;
1-(Cyclohexylmethyl)-6-(4-(oxazol-5-yl)phenyl)-1H-imidazo[4,5-b]pyrazin-2(3H)-one;
6-(2-Methyl-1H-benzo[d]imidazol-5-yl)-1-((tetrahydro-2H-pyran-4-yl)methyl)-1H-imidazo[4,5-b]pyrazin-2(3H)-one hydrocholoride;
6-(4-(5-(Methoxymethyl)-1H-1,2,4-triazol-3-yl)phenyl)-1-((tetrahydro-2H-pyran-4-yl)methyl)-1H-imidazo[4,5-b]pyrazin-2(3H)-one;
1-((1s,4s)-4-(Hydroxymethyl)cyclohexyl)-6-(4-hydroxyphenyl)-1H-imidazo[4,5-b]pyrazin-2(3H)-one;
6-(3-Methyl-1H-pyrazol-4-yl)-1-((tetrahydro-2H-pyran-4-yl)methyl)-1H-imidazo[4,5-b]pyrazin-2(3H)-one;
6-(1H-Pyrazol-4-yl)-1-((tetrahydro-2H-pyran-4-yl)methyl)-1H-imidazo[4,5-b]pyrazin-2(3H)-one;
6-(2-Amino-1H-benzo[d]imidazol-5-yl)-1-((tetrahydro-2H-pyran-4-yl)methyl)-1H-imidazo[4,5-b]pyrazin-2(3H)-one di hydrochloride;
64445-(2-Hydroxypropan-2-yl)-1H-1,2,4-triazol-3-yl)phenyl)-1-((tetrahydro-2H-pyran-4-yl)methyl)-1H-imidazo[4,5-b]pyrazin-2(3H)-one;
6-(4-(5-Isopropyl-1H-1,2,4-triazol-3-yl)phenyl)-1-((tetrahydro-2H-pyran-4-yl)methyl)-1H-imidazo[4,5-b]pyrazin-2(3H)-one;
4-(2-Methoxy-1-(2-morpholinoethyl)-1H-imidazo[4,5-b]pyrazin-6-yl)benzamide hydrochloride;
4-(1-((1s,4s)-4-Hydroxycyclohexyl)-2-methoxy-1H-imidazo[4,5-b]pyrazin-6-yl)benzamide;
6-(4-Hydroxyphenyl)-1-((1s,4s)-4-(methoxymethyl)cyclohexyl)-1H-imidazo[4,5-b]pyrazin-2(3H)-one;
6-(3H-imidazo[4,5-b]pyridin-6-yl)-1-((tetrahydro-2H-pyran-4-yl)methyl)-1H-imidazo[4,5-b]pyrazin-2(3H)-one;
1-(2-(2,2-Dimethyltetrahydro-2H-pyran-4-yl)ethyl)-6-(4-hydroxyphenyl)-1H-imidazo[4,5-b]pyrazin-2(3H)-one;
6-(4-(1H-Pyrazol-1-yl)phenyl)-1-((tetrahydro-2H-pyran-4-yl)methyl)-1H-imidazo[4,5-b]pyrazin-2(3H)-one;
6-(4-(4H-1,2,4-Triazol-3-yl)phenyl)-1-(2-morpholinoethyl)-1H-imidazo[4,5-b]pyrazin-2(3H)-one;
6-(4-(1H-Benzo[d]imidazol-2-yl)phenyl)-1-((tetrahydro-2H-pyran-4-yl)methyl)-1H-imidazo[4,5-b]pyrazin-2(3H)-one;
6-(4-(1H-Imidazol-2-yl)phenyl)-1-((tetrahydro-2H-pyran-4-yl)methyl)-1H-imidazo[4,5-b]pyrazin-2(3H)-one hydrochloride;
6-(4-(5-(Hydroxymethyl)-1H-1,2,4-triazol-3-yl)phenyl)-1-((tetrahydro-2H-pyran-4-yl)methyl)-1H-imidazo[4,5-b]pyrazin-2(3H)-one;
6-(4-(1H-Imidazol-5-yl)phenyl)-1-((tetrahydro-2H-pyran-4-yl)methyl)-1H-imidazo[4,5-b]pyrazin-2(3H)-one hydrochloride;
6-(4-Hydroxyphenyl)-1-((5-oxopyrrolidin-2-yl)methyl)-1H-imidazo[4,5-b]pyrazin-2(3H)-one;
6-(4-(4,5-Dimethyl-1H-imidazol-2-yl)phenyl)-1-((tetrahydro-2H-pyran-4-yl)methyl)-1H-imidazo[4,5-b]pyrazin-2(3H)-one;
6-(4-(1H-1,2,4-Triazol-5-yl)phenyl)-1-(((1s,4s)-4-methoxycyclohexyl)methyl)-1H-imidazo[4,5-b]pyrazin-2(3H)-one;
6-(4-(1H-1,2,4-Triazol-5-yl)phenyl)-1-(((1r,4r)-4-methoxycyclohexyl)methyl)-1H-imidazo[4,5-b]pyrazin-2(3H)-one;
6-(6-(1H-1,2,4-Triazol-3-yl)pyridin-3-yl)-1-((tetrahydro-2H-pyran-4-yl)methyl)-1H-imidazo[4,5-b]pyrazin-2(3H)-one;
6-(4-(1H-1,2,4-Triazol-3-yl)phenyl)-1-(2-(2-oxopyrrolidin-1-yl)ethyl)-1H-imidazo[4,5-b]pyrazin-2(3H)-one;
6-(4-(5-((dimethylamino)methyl)-1H-1,2,4-triazol-3-yl)phenyl)-1-((tetrahydro-2H-pyran-4-yl)methyl)-1H-imidazo[4,5-b]pyrazin-2(3H)-one;
6-(4-Hydroxyphenyl)-1-(pyrrolidin-2-ylmethyl)-1H-imidazo[4,5-b]pyrazin-2(3H)-one hydrochloride;
6-(2-Aminobenzimidazol-5-yl)-1-(cyclohexylmethyl)-4-imidazolino[4,5-b]pyrazin-2-one di hydrochloride;
6-(2-(Dimethylamino)-1H-benzo[d]imidazol-5-yl)-1-((tetrahydro-2H-pyran-4-yl)methyl)-1H-imidazo[4,5-b]pyrazin-2(3H)-one;
6-(4-Hydroxyphenyl)-1-(piperidin-3-ylmethyl)-1H-imidazo[4,5-b]pyrazin-2(3H)-one;
6-(4-(4H-1,2,4-triazol-3-yl)phenyl)-1-(2-(piperidin-1-yl)ethyl)-1H-imidazo[4,5-b]pyrazin-2(3H)-one hydrochloride;
1-(Cyclohexylmethyl)-6-(2-(methylamino)pyrimidin-5-yl)-1H-imidazo[4,5-b]pyrazin-2(3H)-one;
6-(3-methyl-4-(1H-1,2,4-triazol-3-yl)phenyl)-1-((tetrahydro-2H-pyran-4-yl)methyl)-1H-imidazo[4,5-b]pyrazin-2(3H)-one;
1-(Cyclohexylmethyl)-6-(2-(2-methoxyethylamino)pyrimidin-5-yl)-1H-imidazo[4,5-b]pyrazin-2(3H)-one;
6-(4-(5-((methylamino)methyl)-1H-1,2,4-triazol-3-yl)phenyl)-1-((tetrahydro-2H-pyran-4-yl)methyl)-1H-imidazo[4,5-b]pyrazin-2(3H)-one;
6-(4-(5-Oxopyrrolidin-2-yl)phenyl)-1-(2-(tetrahydro-2H-pyran-4-yl)ethyl)-1H-imidazo[4,5-b]pyrazin-2(3H)-one;
6-(4-(5-methyl-1H-1,2,4-triazol-3-yl)phenyl)-1-(2-(tetrahydro-2H-pyran-4-yl)ethyl)-1H-imidazo[4,5-b]pyrazin-2(3H)-one;
6-(4-(1H-imidazol-2-yl)phenyl)-1-(2-(tetrahydro-2H-pyran-4-yl)ethyl)-1H-imidazo[4,5-b]pyrazin-2(3H)-one;
6-(4-(4H-1,2,4-triazol-3-yl)phenyl)-1-(2-methyl-2-morpholinopropyl)-1H-imidazo[4,5-b]pyrazin-2(3H)-one;
6-(4-(4H-1,2,4-Triazol-3-yl)phenyl)-1-(1-morpholinopropan-2-yl)-1H-imidazo[4,5-b]pyrazin-2(3H)-one;
6-(4-(Pyrrolidin-2-yl)phenyl)-1-(2-(tetrahydro-2H-pyran-4-yl)ethyl)-1H-imidazo[4,5-b]pyrazin-2(3H)-one;
6-(4-(5-(aminomethyl)-1H-1,2,4-triazol-3-yl)phenyl)-1-(2-(tetrahydro-2H-pyran-4-yl)ethyl)-1H-imidazo[4,5-b]pyrazin-2(3H)-one;
6-(5-(Hydroxymethyl)thiophen-2-yl)-1-((tetrahydro-2H-pyran-4-yl)methyl)-1H-imidazo[4,5-b]pyrazin-2(3H)-one;
(1r,4s)-4-(6-(4-Hydroxyphenyl)-2-oxo-2,3-dihydro-1H-imidazo[4,5-b]pyrazin-1-yl)cyclo-hexanecarboxamide;
(1s,4s)-4-(6-(4-Hydroxyphenyl)-2-oxo-2,3-dihydro-1H-imidazo[4,5-b]pyrazin-1-yl)cyclohexanecarboxamide;
6-(4-(5-methyl-1H-1,2,4-triazol-3-yl)phenyl)-1-(2-morpholinoethyl)-1H-imidazo[4,5-b]pyrazin-2(3H)-one;
6-(4-(5-Oxopyrrolidin-3-yl)phenyl)-1-(2-(tetrahydro-2H-pyran-4-yl)ethyl)-1H-imidazo[4,5-b]pyrazin-2(3H)-one;
6-(4-(Pyrrolidin-3-yl)phenyl)-1-(2-(tetrahydro-2H-pyran-4-yl)ethyl)-1H-imidazo[4,5-b]pyrazin-2(3H)-one;
6-(1H-benzo[d]imidazol-5-yl)-1-(2-(tetrahydro-2H-pyran-4-yl)ethyl)-1H-imidazo[4,5-b]pyrazin-2(3H)-one;
6-(3-(Hydroxymethyl)thiophen-2-yl)-1-((tetrahydro-2H-pyran-4-yl)methyl)-1H-imidazo[4,5-b]pyrazin-2(3H)-one;
6-5-(2-Hydroxyethyl)thiophen-2-yl)-1-((tetrahydro-2H-pyran-4-yl)methyl)-1H-imidazo[4,5-b]pyrazin-2(3H)-one;
1-(Cyclohexylmethyl)-6-(pyrimidin-5-yl)-1H-imidazo[4,5-b]pyrazin-2(3H)-one;
6-(6-Fluoropyridin-3-yl)-1-((tetrahydro-2H-pyran-4-yl)methyl)-1H-imidazo[4,5-b]pyrazin-2(3H)-one;
6-(6-Aminopyridin-3-yl)-1-((tetrahydro-2H-pyran-4-yl)methyl)-1H-imidazo[4,5-b]pyrazin-2(3H)-one;
6-(4-(5-methyl-1H-imidazol-2-yl)phenyl)-1-((tetrahydro-2H-pyran-4-yl)methyl)-1H-imidazo[4,5-b]pyrazin-2(3H)-one;
6-(4-(5-Methyl-1H-1,2,4-triazol-3-yl)phenyl)-1-(2-(2-oxopyrrolidin-1-yl)ethyl)-1H-imidazo[4,5-b]pyrazin-2(3H)-one;
6-(6-(Methylamino)pyridin-3-yl)-1-((tetrahydro-2H-pyran-4-yl)methyl)-1H-imidazo[4,5-b]pyrazin-2(3H)-one;
6-(2-aminopyrimidin-5-yl)-1-(cyclohexylmethyl)-1H-imidazo[4,5-b]pyrazin-2(3H)-one;
6-(4-(2-hydroxypropan-2-yl)phenyl)-1-(((1r,4r)-4-methoxycyclohexyl)methyl)-1H-imidazo[4,5-b]pyrazin-2(3H)-one;
6-(4-hydroxyphenyl)-1-((1-methylpiperidin-3-yl)methyl)-1H-imidazo[4,5-b]pyrazin-2(3H)-one;
6-(2-methyl-4-(1H-1,2,4-triazol-3-yl)phenyl)-1-(2-(tetrahydro-2H-pyran-4-yl)ethyl)-1H-imidazo[4,5-b]pyrazin-2(3H)-one;
1-(cyclohexylmethyl)-6-(6-(2-hydroxypropan-2-yl)pyridin-3-yl)-1H-imidazo[4,5-b]pyrazin-2(3H)-one;
6-(4-(hydroxymethyl)thiophen-2-yl)-1-((tetrahydro-2H-pyran-4-yl)methyl)-1H-imidazo[4,5-b]pyrazin-2(3H)-one;
6-(1H-benzo[d]imidazol-6-yl)-1-(((1r,4r)-4-methoxycyclohexyl)methyl)-1H-imidazo[4,5-b]pyrazin-2(3H)-one;
6-(4-(4,5-dimethyl-1H-imidazol-2-yl)phenyl)-1-(2-morpholinoethyl)-1H-imidazo[4,5-b]pyrazin-2(3H)-one;
6-(6-(2-hydroxypropan-2-yl)pyridin-3-yl)-1-((tetrahydro-2H-pyran-4-yl)methyl)-1H-imidazo[4,5-b]pyrazin-2(3H)-one;
6-(6-(2-hydroxypropan-2-yl)pyridin-3-yl)-1-(2-(tetrahydro-2H-pyran-4-yl)ethyl)-1H-imidazo[4,5-b]pyrazin-2(3H)-one;
6-(4-(4H-1,2,4-triazol-3-yl)phenyl)-1-(2-morpholino-2-oxoethyl)-1H-imidazo[4,5-b]pyrazin-2(3H)-one;
6-(4-(4H-1,2,4-triazol-3-yl)phenyl)-3-(cyclohexylmethyl)-3,4-dihydropyrazino[2,3-b]pyrazin-2(1H)-one;
6-(4-(1H-1,2,4-triazol-3-yl)phenyl)-1-(2-(tetrahydro-2H-pyran-4-yl)ethyl)-1H-imidazo[4,5-b]pyridin-2(3H)-one;
(R)-6-(4-(1H-1,2,4-triazol-3-yl)phenyl)-1-(1-phenylethyl)-1H-imidazo[4,5-b]pyrazin-2(3H)-one;
(S)-6-(4-(1H-1,2,4-triazol-3-yl)phenyl)-1-(1-phenylethyl)-1H-imidazo[4,5-b]pyrazin-2(3H)-one;
(1r,4r)-4-(6-(4-(2-hydroxypropan-2-yl)phenyl)-2-oxo-2,3-dihydro-1H-imidazo[4,5-b]pyrazin-1-yl)cyclohexanecarboxamide;
6-(3-Methyl-4-(1H-1,2,4-Triazol-3-yl)phenyl)-1-((tetrahydro-2H-pyran-4-yl)methyl)-1H-imidazo[4,5-B]pyrazin-2(3H)-one;
6-(4-(1H-imidazol-2-yl)phenyl)-1-(2-(tetrahydro-2H-pyran-4-yl)ethyl)-1H-imidazo[4,5-b]pyrazin-2(3H)-one;
6-(4-(5-(Aminomethyl)-1H-1,2,4-triazol-3-yl)phenyl)-1-(2-(tetrahydro-2H-pyran-4-yl)ethyl)-1H-imidazo[4,5-b]pyrazin-2(3H)-one;
6-(1H-benzo[d]imidazol-5-yl)-1-(2-(tetrahydro-2H-pyran-4-yl)ethyl)-1H-imidazo[4,5-b]pyrazin-2(3H)-one;
6-(2-Aminopyrimidin-5-yl)-1-(cyclohexylmethyl)-1H-imidazo[4,5-b]pyrazin-2(3H)-one;
6-(4-Hydroxyphenyl)-1-((1-methylpiperidin-2-yl)methyl)-1H-imidazo[4,5-b]pyrazin-2(3H)-one hydrochloride;
6-(3-Methyl-4-(1H-1,2,4-Triazol-3-yl)phenyl)-1-((tetrahydro-2H-pyran-4-yl)methyl)-1H-imidazo[4,5-B]pyrazin-2(3H)-one;
1-(Cyclohexylmethyl)-6-(6-(2-hydroxypropan-2-yl)pyridin-3-yl)-1H-imidazo[4,5-b]pyrazin-2(3H)-one;
6-(6-(2-Hydroxypropan-2-yl)pyridin-3-yl)-1-((tetrahydro-2H-pyran-4-yl)methyl)-1H-imidazo[4,5-b]pyrazin-2(3H)-one;
6-(6-(2-Hydroxypropan-2-yl)pyridin-3-yl)-1-(2-(tetrahydro-2H-pyran-4-yl)ethyl)-1H-imidazo[4,5-b]pyrazin-2(3H)-one;
6-(4-(4H-1,2,4-Triazol-3-yl)phenyl)-1-(2-morpholino-2-oxoethyl)-1H-imidazo[4,5-b]pyrazin-2(3H)-one;
(R)-6-(4-(4H-1,2,4-Triazol-3-yl)phenyl)-3-(cyclohexylmethyl)-3,4-dihydropyrazino[2,3-b]pyrazin-2(1H)-one;
(R)-6-(4-(1H-1,2,4-Triazol-3-yl)phenyl)-1-(1-phenylethyl)-1H-imidazo[4,5-B]pyrazin-2(3H)-one;
(S)-6-(4-(4H-1,2,4-Triazol-3-yl)phenyl)-1-(1-phenylethyl)-1H-imidazo[4,5-b]pyrazin-2(3H)-one;
(1r,4s)-4-(6-(4-(2-Hydroxypropan-2-yl)phenyl)-2-oxo-2,3-dihydro-1H-imidazo[4,5-b]pyrazin-1-yl)cyclohexanecarboxamide; and
6-(4-(5-Methyl-1H-1,2,4-triazol-3-yl)phenyl)-1-(2-(tetrahydro-2H-pyran-4-yl)ethyl)-1H-imidazo[4,5-b]pyrazin-2(3H)-one,
   and pharmaceutically acceptable salts, clathrates, solvates, stereoisomers, tautomers, and prodrugs thereof.

In one embodiment, the TOR kinase inhibitors include compounds having the following formula (II):

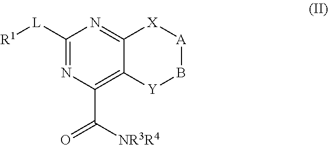

and pharmaceutically acceptable salts, clathrates, solvates, stereoisomers, tautomers, and prodrugs thereof, wherein:

R¹ is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl;

—X-A-B—Y— taken together form —N(R²)CH₂C(O)NH—, —N(R²)C(O)CH₂NH—, —N(R²)C(O)NH—, —N(R²)C═N—, or —C(R²)═CHNH—;

L is a direct bond, NH or O;

R² is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl; and

R³ and R⁴ are independently H or C₁₋₈alkyl.

In one embodiment, the TOR kinase inhibitors of formula (II) are those wherein —X-A-B—Y— taken together form —N(R²)CH₂C(O)NH—.

In another embodiment, the TOR kinase inhibitors of formula (II) are those wherein —X-A-B—Y— taken together form —N(R²)C(O)CH₂NH—.

In another embodiment, the TOR kinase inhibitors of formula (II) are those wherein —X-A-B—Y— taken together form —N(R²)C(O)NH—.

In another embodiment, the TOR kinase inhibitors of formula (II) are those wherein —X-A-B—Y— taken together form —N(R²)C═N—.

In another embodiment, the TOR kinase inhibitors of formula (II) are those wherein —X-A-B—Y— taken together form —C(R²)═CHNH—.

In another embodiment, the TOR kinase inhibitors of formula (II) are those wherein L is a direct bond.

In another embodiment, the TOR kinase inhibitors of formula (II) are those wherein R¹ is substituted aryl, such as substituted phenyl.

In another embodiment, the TOR kinase inhibitors of formula (II) are those wherein R¹ is substituted or unsubstituted heteroaryl, such as substituted or unsubstituted pyridine, substituted or unsubstituted indole or substituted or unsubstituted quinoline.

In another embodiment, the TOR kinase inhibitors of formula (II) are those wherein R¹ is substituted or unsubstituted cycloalkyl, such as substituted or unsubstituted cyclopentyl.

In another embodiment, the TOR kinase inhibitors of formula (II) are those wherein —X-A-B—Y— taken together form —N(R²)C(O)NH— and R¹ is substituted aryl, such as phenyl.

In another embodiment, the TOR kinase inhibitors of formula (II) are those wherein —X-A-B—Y— taken together form —N(R²)C(O)NH— and R¹ is substituted or unsubstituted heteroaryl, such as substituted or unsubstituted pyridine, substituted or unsubstituted indole or substituted or unsubstituted quinoline.

In another embodiment, the TOR kinase inhibitors of formula (II) are those wherein —X-A-B—Y— taken together form —N(R²)C(O)NH— and R¹ is substituted or unsubstituted cycloalkyl, such as substituted or unsubstituted cyclopentyl.

In another embodiment, the TOR kinase inhibitors of formula (II) are those wherein R² is substituted C₁₋₈alkyl, such as —CH₂C₆H₅.

In another embodiment, the TOR kinase inhibitors of formula (II) are those wherein R² is unsubstituted C₁₋₈alkyl, such as unsubstituted methyl.

In another embodiment, the TOR kinase inhibitors of formula (II) are those wherein R² is substituted or unsubstituted aryl, such as substituted or unsubstituted phenyl.

In another embodiment, the TOR kinase inhibitors of formula (II) are those wherein R² is substituted aryl, such as halo, haloalkyl or alkoxy substituted phenyl.

In another embodiment, the TOR kinase inhibitors of formula (II) are those wherein R² is substituted or unsubstituted cycloalkyl, such as substituted or unsubstituted cyclohexyl or substituted or unsubstituted cycloheptyl.

In another embodiment, the TOR kinase inhibitors of formula (II) are those wherein R² is substituted heterocyclylalkyl, such as substituted piperidine.

In another embodiment, the TOR kinase inhibitors of formula (II) are those wherein R³ and R⁴ are H.

In another embodiment, the TOR kinase inhibitors of formula (II) are those wherein —X-A-B—Y— taken together form —N(R²)C(O)NH— and R² is unsubstituted aryl, such as unsubstituted phenyl.

In another embodiment, the TOR kinase inhibitors of formula (II) are those wherein —X-A-B—Y— taken together form —N(R²)C(O)NH—, R¹ is substituted or unsubstituted heteroaryl, such as substituted or unsubstituted pyridine, and R² is substituted or unsubstituted aryl, such as substituted or unsubstituted phenyl.

In another embodiment, the TOR kinase inhibitors of formula (II) are those wherein —X-A-B—Y— taken together form —N(R²)C(O)NH—, R¹ is substituted or unsubstituted heteroaryl, such as substituted or unsubstituted pyridine, R² is substituted or unsubstituted aryl, such as substituted or unsubstituted phenyl, and R³ and R⁴ are H.

In another embodiment, the TOR kinase inhibitors of formula (II) are those wherein —X-A-B—Y— taken together form —N(R²)C(O)NH—, L is a direct bond, R¹ is substituted or unsubstituted heteroaryl, such as substituted or unsubstituted pyridine, R² is substituted or unsubstituted aryl, such as substituted or unsubstituted phenyl, and R³ and R⁴ are H.

In another embodiment, the TOR kinase inhibitors of formula (II) are those wherein —X-A-B—Y— taken together form —N(R²)C(O)NH—, R¹ is substituted or unsubstituted aryl, such as substituted or unsubstituted phenyl, and R² is substituted or unsubstituted aryl, such as substituted or unsubstituted phenyl.

In another embodiment, the TOR kinase inhibitors of formula (II) are those wherein —X-A-B—Y— taken together form —N(R²)C(O)NH—, R¹ is substituted or unsubstituted aryl, such as substituted or unsubstituted phenyl, R² is substituted or unsubstituted aryl, such as substituted or unsubstituted phenyl, and R³ and R⁴ are H.

In another embodiment, the TOR kinase inhibitors of formula (II) are those wherein —X-A-B—Y— taken together form —N(R²)C(O)NH—, L is a direct bond, R¹ is substituted or unsubstituted aryl, such as substituted or unsubstituted phenyl, R² is substituted or unsubstituted aryl, such as substituted or unsubstituted phenyl, and R³ and R⁴ are H.

In another embodiment, the TOR kinase inhibitors of formula (II) are those wherein —X-A-B—Y— taken together form —N(R²)C(O)NH—, R¹ is substituted or unsubstituted heteroaryl, L is a direct bond and R² is substituted or unsubstituted C₁₋₈alkyl or substituted or unsubstituted cycloalkyl.

In another embodiment, the TOR kinase inhibitors of formula (II) are those wherein —X-A-B—Y— taken together form —N(R²)C(O)NH—, R¹ is substituted or unsubstituted aryl, L is a direct bond and R² is substituted or unsubstituted C₁₋₈alkyl or substituted or unsubstituted cycloalkyl.

In another embodiment, the TOR kinase inhibitors of formula (II) do not include 8,9-dihydro-8-oxo-9-phenyl-2-(3-pyridinyl)-7H-purine-6-carboxamide, 8,9-dihydro-8-oxo-9-phenyl-2-(3-pyridinyl)-7H-purine-6-carboxamide, 8,9-dihydro-8-oxo-9-phenyl-2-(3-pyridinyl)-7H-purine-6-carboxamide, 2-(4-cyanophenyl)-8-oxo-9-phenyl-8,9-dihydro-7H-purine-6-carboxamide, 2-(4-nitrophenyl)-8-oxo-9-phenyl-8,9-dihydro-7H-purine-6-carboxamide, 9-benzyl-2-(4-methoxyphenyl)-8-oxo-8,9-dihydro-7H-purine-6-carboxamide, 2-methyl-8-oxo-9-phenyl-8,9-dihydro-7H-purine-6-carboxamide, 9-benzyl-9H-purine-2,6-dicarboxamide, 9-[2,3-bis[(benzoyloxy)methyl]cyclobutyl]-2-methyl-9H-Purine-6-carboxamide, 9-benzyl-2-methyl-9H-purine-6-carboxamide, 9-(2-hydroxyethyl)-2-methyl-9H-purine-6-carboxamide, 9-(2-hydroxyethyl)-2-(trifluoromethyl)-9H-purine-6-carboxamide, 9-(2-hydroxyethyl)-2-(prop-1-enyl)-9H-purine-6-carboxamide, 9-(2-hydroxyethyl)-2-phenyl-9H-purine-6-carboxamide, 9-(3-hydroxypropyl)-2-methyl-9H-purine-6-carboxamide, 9-(3-hydroxypropyl)-2-(trifluoromethyl)-9H-purine-6-carboxamide, 2-methyl-9-phenylmethyl-9H-purine-6-carboxamide or 2-methyl-9-β-D-ribofuranosyl-9H-purine-6-carboxamide.

In another embodiment, the TOR kinase inhibitors of formula (II) do not include compounds wherein R² is a substituted furanoside.

In another embodiment, the TOR kinase inhibitors of formula (II) do not include compounds wherein R² is a substituted or unsubstituted furanoside.

In another embodiment, the TOR kinase inhibitors of formula (II) do not include (2′R)-2′-deoxy-2′-fluoro-2′-C-methyl nucleosides.

In one embodiment, the TOR kinase inhibitors include compounds having the following formula (IIa):

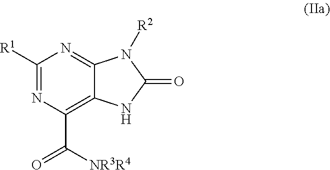

and pharmaceutically acceptable salts, clathrates, solvates, stereoisomers, tautomers, and prodrugs thereof, wherein:

R¹ is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl;

R² is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl; and

R³ and R⁴ are independently H or C₁₋₈alkyl.

In one embodiment, the TOR kinase inhibitors of formula (IIa) are those wherein

R¹ is substituted aryl, substituted or unsubstituted heteroaryl, such as substituted phenyl.

In another embodiment, the TOR kinase inhibitors of formula (IIa) are those wherein R¹ is substituted or unsubstituted heteroaryl, such as substituted or unsubstituted pyridine, substituted or unsubstituted indole or substituted or unsubstituted quinoline.

In another embodiment, the TOR kinase inhibitors of formula (IIa) are those wherein R¹ is substituted or unsubstituted cycloalkyl, such as substituted or unsubstituted cyclopentyl.

In another embodiment, the TOR kinase inhibitors of formula (IIa) are those wherein R² is substituted C₁₋₈alkyl, such as —CH₂C₆H₅.

In another embodiment, the TOR kinase inhibitors of formula (IIa) are those wherein R² is unsubstituted C₁₋₈alkyl, such as unsubstituted methyl.

In another embodiment, the TOR kinase inhibitors of formula (IIa) are those wherein R² is substituted or unsubstituted aryl, such as substituted or unsubstituted phenyl.

In another embodiment, the TOR kinase inhibitors of formula (IIa) are those wherein R² is substituted aryl, such as halo, haloalkyl or alkoxy substituted phenyl.

In another embodiment, the TOR kinase inhibitors of formula (IIa) are those wherein R² is substituted or unsubstituted cycloalkyl, such as substituted or unsubstituted cyclohexyl or substituted or unsubstituted cycloheptyl.

In another embodiment, the TOR kinase inhibitors of formula (IIa) are those wherein R² is substituted heterocyclylalkyl, such as substituted piperidine.

In another embodiment, the TOR kinase inhibitors of formula (IIa) are those wherein R³ and R⁴ are H.

In another embodiment, the TOR kinase inhibitors of formula (IIa) do not include 8,9-dihydro-8-oxo-9-phenyl-2-(3-pyridinyl)-7H-Purine-6-carboxamide, 8,9-dihydro-8-oxo-9-phenyl-2-(3-pyridinyl)-7H-Purine-6-carboxamide, 8,9-dihydro-8-oxo-9-phenyl-2-(3-pyridinyl)-7H-Purine-6-carboxamide, 2-(4-cyanophenyl)-8-oxo-9-phenyl-8,9-dihydro-7H-purine-6-carboxamide, 2-(4-nitrophenyl)-8-oxo-9-phenyl-8,9-dihydro-7H-purine-6-carboxamide, 9-benzyl-2-(4-methoxyphenyl)-8-oxo-8,9-dihydro-7H-purine-6-carboxamide, 9-phenylmethyl-9H-purine-2,6-dicarboxamide, or 2-methyl-8-oxo-9-phenyl-8,9-dihydro-7H-purine-6-carboxamide.

In another embodiment, the TOR kinase inhibitors of formula (IIa) do not include compounds wherein R² is a substituted furanoside.

In another embodiment, the TOR kinase inhibitors of formula (IIa) do not include compounds wherein R² is a substituted or unsubstituted furanoside.

In another embodiment, the TOR kinase inhibitors of formula (IIa) do not include (2′R)-2′-deoxy-2′-fluoro-2′-C-methyl nucleosides.

In one embodiment, the TOR kinase inhibitors include compounds having the following formula (IIb):

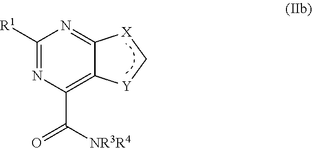

and pharmaceutically acceptable salts, clathrates, solvates, stereoisomers, tautomers, and prodrugs thereof, wherein:

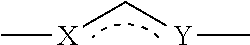

### is —C(R²)═CH—NH— or —N(R²)—CH═N—;

R¹ is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl;

R² is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl; and

R³ and R⁴ are independently H or C₁₋₈alkyl.

In one embodiment, the TOR kinase inhibitors of formula (IIb) are those wherein R¹ is substituted aryl, such as substituted phenyl.

In another embodiment, the TOR kinase inhibitors of formula (IIb) are those wherein R¹ is substituted or unsubstituted heteroaryl, such as substituted or unsubstituted pyridine, substituted or unsubstituted indole or substituted or unsubstituted quinoline.

In another embodiment, the TOR kinase inhibitors of formula (IIb) are those wherein R¹ is substituted or unsubstituted cycloalkyl, such as substituted or unsubstituted cyclopentyl.

In another embodiment, the TOR kinase inhibitors of formula (IIb) are those wherein R² is substituted C₁₋₈alkyl, such as —CH₂C₆H₅.

In another embodiment, the TOR kinase inhibitors of formula (IIb) are those wherein R² is unsubstituted C₁₋₈alkyl, such as unsubstituted methyl.

In another embodiment, the TOR kinase inhibitors of formula (IIb) are those wherein R² is substituted or unsubstituted aryl, such as substituted or unsubstituted phenyl.

In another embodiment, the TOR kinase inhibitors of formula (IIb) are those wherein R² is substituted aryl, such as halo, haloalkyl or alkoxy substituted phenyl.

In another embodiment, the TOR kinase inhibitors of formula (IIb) are those wherein R² is substituted or unsubstituted cycloalkyl, such as substituted or unsubstituted cyclohexyl or substituted or unsubstituted cycloheptyl.

In another embodiment, the TOR kinase inhibitors of formula (IIb) are those wherein R² is substituted heterocyclylalkyl, such as substituted piperidine.

In another embodiment, the TOR kinase inhibitors of formula (IIb) are those wherein R³ and R⁴ are H.

In another embodiment, the TOR kinase inhibitors of formula (IIb) are those wherein

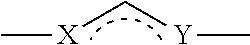

is —C(R²)═CH—NH— and R² is substituted aryl, such as substituted phenyl.

In another embodiment, the TOR kinase inhibitors of formula (IIb) are those wherein

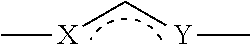

is —N(R²)—CH═N— and R² is substituted aryl, such as substituted phenyl.

In another embodiment, the TOR kinase inhibitors of formula (IIb) are those wherein R¹ is substituted aryl, such as phenyl, and R² is substituted aryl, such as substituted phenyl.

In another embodiment, the TOR kinase inhibitors of formula (IIb) do not include 9-benzyl-9H-purine-2,6-dicarboxamide, 9-[2,3-bis[(benzoyloxy)methyl]cyclobutyl]-2-methyl-9H-Purine-6-carboxamide, 9-benzyl-2-methyl-9H-purine-6-carboxamide, 9-(2-hydroxyethyl)-2-methyl-9H-purine-6-carboxamide, 9-(2-hydroxyethyl)-2-(trifluoromethyl)-9H-purine-6-carboxamide, 9-(2-hydroxyethyl)-2-(prop-1-enyl)-9H-purine-6-carboxamide, 9-(2-hydroxyethyl)-2-phenyl-9H-purine-6-carboxamide, 9-(3-hydroxypropyl)-2-methyl-9H-purine-6-carboxamide, 9-(3-hydroxypropyl)-2-(trifluoromethyl)-9H-purine-6-carboxamide, 9-phenylmethyl-9H-purine-2,6-dicarboxamide, 2-methyl-9-phenylmethyl-9H-purine-6-carboxamide or 2-methyl-9-β-D-ribofuranosyl-9H-purine-6-carboxamide.

In another embodiment, the TOR kinase inhibitors of formula (IIb) do not include compounds wherein R² is substituted cyclobutyl when

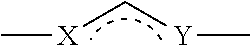

### is —N(R²)—CH═N—.

In another embodiment, the TOR kinase inhibitors of formula (IIb) do not include compounds wherein R² is a substituted furanoside when

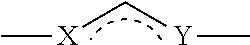

### is —N(R²)—CH═N—.

In another embodiment, the TOR kinase inhibitors of formula (IIb) do not include compounds wherein R² is substituted pyrimidine when

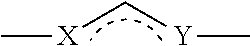

### is —C(R²)═CH—NH—.

In another embodiment, the TOR kinase inhibitors of formula (IIb) do not include compounds wherein R² is substituted oxetane when

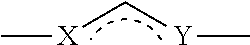

### is —N(R²)—CH═N—.

In another embodiment, the TOR kinase inhibitors of formula (IIb) do not include compounds wherein R² is substituted cyclopentyl or a heterocyclopentyl when

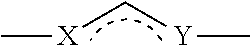

### is —N(R²)—CH═N—.

In one embodiment, the TOR kinase inhibitors include compounds having the following formula (IIc):

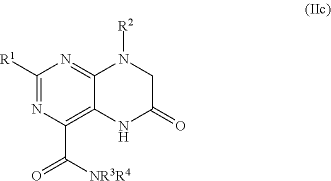

and pharmaceutically acceptable salts, clathrates, solvates, stereoisomers, tautomers, and prodrugs thereof, wherein:

R¹ is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl;

R² is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl; and

R³ and R⁴ are independently H or C₁₋₈alkyl.

In one embodiment, the TOR kinase inhibitors of formula (IIc) are those wherein R¹ is substituted aryl, such as substituted phenyl.

In another embodiment, the TOR kinase inhibitors of formula (IIc) are those wherein R¹ is substituted or unsubstituted heteroaryl, such as substituted or unsubstituted pyridine, substituted or unsubstituted indole or substituted or unsubstituted quinoline.

In another embodiment, the TOR kinase inhibitors of formula (IIc) are those wherein R¹ is substituted or unsubstituted cycloalkyl, such as substituted or unsubstituted cyclopentyl.

In another embodiment, the TOR kinase inhibitors of formula (IIc) are those wherein R² is substituted C₁₋₈alkyl, such as —CH₂C₆H₅.

In another embodiment, the TOR kinase inhibitors of formula (IIc) are those wherein R² is unsubstituted C₁₋₈alkyl, such as unsubstituted methyl.

In another embodiment, the TOR kinase inhibitors of formula (IIc) are those wherein R² is substituted or unsubstituted aryl, such as substituted or unsubstituted phenyl.

In another embodiment, the TOR kinase inhibitors of formula (IIc) are those wherein R² is substituted aryl, such as halo, haloalkyl or alkoxy substituted phenyl.

In another embodiment, the TOR kinase inhibitors of formula (IIc) are those wherein R² is substituted or unsubstituted cycloalkyl, such as substituted or unsubstituted cyclohexyl or substituted or unsubstituted cycloheptyl.

In another embodiment, the TOR kinase inhibitors of formula (IIc) are those wherein R² is substituted heterocyclylalkyl, such as substituted piperidine.

In another embodiment, the TOR kinase inhibitors of formula (IIc) are those wherein R³ and R⁴ are H.

In one embodiment, the TOR kinase inhibitors include compounds having the following formula (IId):

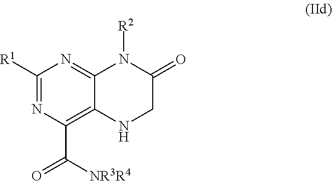

and pharmaceutically acceptable salts, clathrates, solvates, stereoisomers, tautomers, and prodrugs thereof, wherein:

R¹ is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl;

R² is substituted or unsubstituted C₁₋₈alkyl, substituted or unsubstituted aryl, substituted or unsubstituted heteroaryl, substituted or unsubstituted cycloalkyl, or substituted or unsubstituted heterocyclylalkyl; and

R³ and R⁴ are independently H or C₁₋₈alkyl.

In one embodiment, the TOR kinase inhibitors of formula (IId) are those wherein R¹ is substituted aryl, such as substituted phenyl.

In another embodiment, the TOR kinase inhibitors of formula (IId) are those wherein R¹ is substituted or unsubstituted heteroaryl, such as substituted or unsubstituted pyridine, substituted or unsubstituted indole or substituted or unsubstituted quinoline.

In another embodiment, the TOR kinase inhibitors of formula (IId) are those wherein R¹ is substituted or unsubstituted cycloalkyl, such as substituted or unsubstituted cyclopentyl.

In another embodiment, the TOR kinase inhibitors of formula (IId) are those wherein R² is substituted C₁₋₈alkyl, such as —CH₂C₆H₅.

In another embodiment, the TOR kinase inhibitors of formula (IId) are those wherein R² is unsubstituted C₁₋₈alkyl, such as unsubstituted methyl.

In another embodiment, the TOR kinase inhibitors of formula (IId) are those wherein R² is substituted or unsubstituted aryl, such as substituted or unsubstituted phenyl.

In another embodiment, the TOR kinase inhibitors of formula (IId) are those wherein R² is substituted aryl, such as halo, haloalkyl or alkoxy substituted phenyl.

In another embodiment, the TOR kinase inhibitors of formula (IId) are those wherein R² is substituted or unsubstituted cycloalkyl, such as substituted or unsubstituted cyclohexyl or substituted or unsubstituted cycloheptyl.

In another embodiment, the TOR kinase inhibitors of formula (IId) are those wherein R² is substituted heterocyclylalkyl, such as substituted piperidine.

In another embodiment, the TOR kinase inhibitors of formula (IId) are those wherein R³ and R⁴ are H.

Representative TOR kinase inhibitors of formula (II) include:
9-benzyl-8-oxo-2-(pyridin-3-yl)-8,9-dihydro-7H-purine-6-carboxamide;
N-methyl-8-oxo-9-phenyl-2-(pyridin-3-yl)-8,9-dihydro-7H-purine-6-carboxamide;
8-oxo-9-phenyl-2-(pyridin-2-yl)-8,9-dihydro-7H-purine-6-carboxamide;
2-(2-chloropyridin-3-yl)-8-oxo-9-phenyl-8,9-dihydro-7H-purine-6-carboxamide;
2-(2-methoxypyridin-3-yl)-8-oxo-9-phenyl-8,9-dihydro-7H-purine-6-carboxamide;
N,N-dimethyl-8-oxo-9-phenyl-2-(pyridin-3-yl)-8,9-dihydro-7H-purine-6-carboxamide;
9-methyl-8-oxo-2-(pyridin-3-yl)-8,9-dihydro-7H-purine-6-carboxamide;
2-(4-hydroxyphenyl)-9-(2-methoxyphenyl)-8-oxo-8,9-dihydro-7H-purine-6-carboxamide;
2-(3-hydroxyphenyl)-8-oxo-9-o-tolyl-8,9-dihydro-7H-purine-6-carboxamide;
2-(1H-indol-4-yl)-9-(2-methoxyphenyl)-8-oxo-8,9-dihydro-7H-purine-6-carboxamide;
2-(1H-indol-6-yl)-9-(2-methoxyphenyl)-8-oxo-8,9-dihydro-7H-purine-6-carboxamide;
2-(3-hydroxyphenyl)-9-(4-methoxyphenyl)-8-oxo-8,9-dihydro-7H-purine-6-carboxamide;
2-(2-hydroxypyridin-4-yl)-9-(2-methoxyphenyl)-8-oxo-8,9-dihydro-7H-purine-6-carboxamide;
9-(2-chlorophenyl)-2-(3-hydroxyphenyl)-8-oxo-8,9-dihydro-7H-purine-6-carboxamide;
9-(2-fluorophenyl)-2-(3-hydroxyphenyl)-8-oxo-8,9-dihydro-7H-purine-6-carboxamide;
9-(2,6-difluorophenyl)-2-(3-hydroxyphenyl)-8-oxo-8,9-dihydro-7H-purine-6-carboxamide;
9-cycloheptyl-8-oxo-2-(pyridin-3-yl)-8,9-dihydro-7H-purine-6-carboxamide;
9-(2-methoxyphenyl)-8-oxo-2-(quinolin-5-yl)-8,9-dihydro-7H-purine-6-carboxamide;
2-cyclopentyl-9-(2-methoxyphenyl)-8-oxo-8,9-dihydro-7H-purine-6-carboxamide;
9-(2-methoxyphenyl)-8-oxo-2-(3-(trifluoromethyl)phenyl)-8,9-dihydro-7H-purine-6-carboxamide;
9-(2-methoxyphenyl)-2-(6-methoxypyridin-3-yl)-8-oxo-8,9-dihydro-7H-purine-6-carboxamide;
2-(3-hydroxyphenyl)-8-oxo-9-(4-(trifluoromethyl)phenyl)-8,9-dihydro-7H-purine-6-carboxamide;
9-benzyl-2-(3-hydroxyphenyl)-8-oxo-8,9-dihydro-7H-purine-6-carboxamide;
2-(3-hydroxyphenyl)-8-oxo-9-(2-(trifluoromethoxy)phenyl)-8,9-dihydro-7H-purine-6-carboxamide;
9-(2,4-dichlorophenyl)-2-(3-hydroxyphenyl)-8-oxo-8,9-dihydro-7H-purine-6-carboxamide;
9-(2-methoxyphenyl)-2-(3-nitrophenyl)-8-oxo-8,9-dihydro-7H-purine-6-carboxamide;
2-(3-cyanophenyl)-8-oxo-9-phenyl-8,9-dihydro-7H-purine-6-carboxamide;
9-(3-fluorophenyl)-2-(3-hydroxyphenyl)-8-oxo-8,9-dihydro-7H-purine-6-carboxamide;
9-(2-methoxyphenyl)-8-oxo-2-(2-(trifluoromethyl)phenyl)-8,9-dihydro-7H-purine-6-carboxamide;
2-(5-fluoropyridin-3-yl)-9-(2-methoxyphenyl)-8-oxo-8,9-dihydro-7H-purine-6-carboxamide;
2-(1-benzylpiperidin-4-yl)-9-(2-methoxyphenyl)-8-oxo-8,9-dihydro-7H-purine-6-carboxamide; benzyl 4-(6-carbamoyl-8-oxo-2-(pyridin-3-yl)-7H-purin-9(8H)-yl)piperidine-1-carboxylate;
9-cyclohexyl-2-(3-hydroxyphenyl)-8-oxo-8,9-dihydro-7H-purine-6-carboxamide;
9-(2-methoxyphenyl)-8-oxo-2-(3-(trifluoromethoxy)phenyl)-8,9-dihydro-7H-purine-6-carboxamide;
9-phenyl-2-(pyridin-3-yl)-9H-purine-6-carboxamide;
6-oxo-8-phenyl-2-(pyridin-3-yl)-5,6,7,8-tetrahydropteridine-4-carboxamide;
6-oxo-8-phenyl-2-(pyridin-4-yl)-5,6,7,8-tetrahydropteridine-4-carboxamide;
2-(3-aminophenyl)-9-(2-methoxyphenyl)-8-oxo-8,9-dihydro-7H-purine-6-carboxamide;
2-(3-hydroxyphenyl)-9-(2-methoxyphenyl)-9H-purine-6-carboxamide;
9-Cyclopentyl-2-(3-hydroxyphenyl)-8-oxo-8,9-dihydro-7H-purine-6-carboxamide;
9-tert-Butyl-2-(3-hydroxy-phenyl)-8-oxo-8,9-dihydro-7H-purine-6-carboxamide;
[2-(3-Hydroxyphenyl)-9-(2-methoxyphenyl)-8-oxo(7-hydropurin-6-yl)]-N-methylcarbox-amide;
2-phenyl-5H-pyrrolo[3,2-d]pyrimidine-4-carboxamide;
[2-(3-Hydroxyphenyl)-9-(2-methoxyphenyl)-8-oxo(7-hydropurin-6-yl)]-N,N-dimethyl carboxamide;
2-(3-Hydroxyphenylamino)-9-(2-methoxyphenyl)-8-oxo-8,9-dihydro-7H-purine-6-carboxamide;
2-(4-Hydroxyphenylamino)-9-(2-methoxyphenyl)-8-oxo-8,9-dihydro-7H-purine-6-carboxamide;
9-(trans-4-Hydroxycyclohexyl)-2-(3-hydroxyphenyl)-8-oxo-8,9-dihydro-7H-purine-6-carboxamide;
9-(trans-4-Hydroxycyclohexyl)-8-oxo-2-(pyridin-3-yl)-8,9-dihydro-7H-purine-6-carboxamide;
9-(trans-4-Hydroxycyclohexyl)-2-(3-hydroxyphenyl)-8-oxo-8,9-dihydro-7H-purine-6-carboxamide;
9-(trans-4-Hydroxycyclohexyl)-8-oxo-2-(pyridin-3-yl)-8,9-dihydro-7H-purine-6-carboxamide;
2-(3-Hydroxyphenylamino)-9-(2-methoxyphenyl)-9H-purine-6-carboxamide;
9-Isopropyl-2-(3-hydroxy-phenyl)-8-oxo-8,9-dihydro-7H-purine-6-carboxamide;
Methyl 4-(6-carbamoyl-9-(2-methoxyphenyl)-8-oxo-8,9-dihydro-7H-purin-2-yl)benzoate;
2-(2-Chloro-3-hydroxyphenyl)-9-(2-methoxyphenyl)-8-oxo-7-hydropurine-6-carbox amide;
2-(3-Cyanophenyl)-9-(2-methoxyphenyl)-8-oxo-8,9-dihydro-7H-purine-6-carboxamide;
2-(2-Hydroxyphenylamino)-9-(2-methoxyphenyl)-8-oxo-8,9-dihydro-7H-purine-6-carboxamide;
2-(3-Hydroxyphenyl)-9-(4-methoxy-2-methylphenyl)-8-oxo-8,9-dihydro-7H-purine-6-carboxamide;
2-(3-Hydroxyphenyl)-8-oxo-9-(2-(trifluoromethyl)phenyl)-8,9-dihydro-7H-purine-6-carboxamide;
2-(4-Cyano-phenyl)-9-(2-methoxy-phenyl)-8-oxo-8,9-dihydro-7H-purine-6-carboxamide;
4-[6-Carbamoyl-9-(2-methoxy-phenyl)-8-oxo-8,9-dihydro-7H-purin-2-yl]-benzoic acid;
Methyl 3-(6-carbamoyl-9-(2-methoxyphenyl)-8-oxo-8,9-dihydro-7H-purin-2-yl)benzoate;
3-(6-Carbamoyl-9-(2-methoxyphenyl)-8-oxo-8,9-dihydro-7H-purin-2-yl)benzoic acid;
2-(3-Hydroxyphenyl)-9-(2-isopropylphenyl)-8-oxo-8,9-dihydro-7H-purine-6-carboxamide;
2-(1H-Indazol-6-yl)-9-(2-methoxyphenyl)-8-oxo-7-hydropurine-6-carboxamide;
2-(4-Carbamoylphenyl)-9-(2-methoxyphenyl)-8-oxo-8,9-dihydro-7H-purine-6-carboxamide;
9-(2-Ethylphenyl)-2-(3-hydroxyphenyl)-8-oxo-8,9-dihydro-7H-purine-6-carboxamide;
9-(2,5-Dichlorophenyl)-2-(3-hydroxyphenyl)-8-oxo-7-hydropurine-6-carboxamide;
2-(3-Carbamoylphenyl)-9-(2-methoxyphenyl)-8-oxo-8,9-dihydro-7H-purine-6-carbox amide;
9-(2,6-Dichlorophenyl)-2-(3-hydroxyphenyl)-8-oxo-7-hydropurine-6-carboxamide;
2-(2-Hydroxyphenyl)-9-(2-methoxyphenyl)purine-6-carboxamide;
2-(1H-Indazol-5-yl)-9-(2-methoxyphenyl)-8-oxo-7-hydropurine-6-carboxamide;
9-(2,3-Dichlorophenyl)-2-(3-hydroxyphenyl)-8-oxo-7-hydropurine-6-carboxamide;
2-[4-(Hydroxymethyl)phenyl]-9-(2-methoxyphenyl)-8-oxo-7-hydropurine-6-carbox-amide;
2-[3-(Hydroxymethyl)phenyl]-9-(2-methoxyphenyl)-8-oxo-7-hydropurine-6-carbox-amide;
9-(2-Methoxyphenyl)-8-oxo-2-(pyridin-4-yl)-8,9-dihydro-7H-purine-6-carboxamide;
2-(4-Fluoro-3-hydroxyphenyl)-9-(2-methoxyphenyl)-8-oxo-7-hydropurine-6-carbox-amide;
2-(2-Fluoro-3-hydroxyphenyl)-9-(2-methoxyphenyl)-8-oxo-7-hydropurine-6-carbox-amide;
2-[4-(1-Hydroxy-isopropyl)phenyl]-9-(2-methoxyphenyl)-8-oxo-7-hydropurine-6-carboxamide;
2-[3-(1-Hydroxy-isopropyl)phenyl]-9-(2-methoxyphenyl)-8-oxo-7-hydropurine-6-carboxamide;
9-(2-Methoxyphenyl)-2-(2-nitrophenyl)-8-oxo-7-hydropurine-6-carboxamide;
9-(2-Methoxyphenyl)-2-(4-nitrophenyl)-8-oxo-7-hydropurine-6-carboxamide;
9-(2-Methoxyphenyl)-2-(2-nitrophenyl)-8-oxo-7-hydropurine-6-carboxamide;
9-(2,4-Difluorophenyl)-2-(3-hydroxyphenyl)-8-oxo-7-hydropurine-6-carboxamide;
9-(2-Methoxyphenyl)-2-{3-[(methylsulfonyl)amino]phenyl}-8-oxo-7-hydropurine-6-carboxamide;
9-(4-Chloro-2-fluorophenyl)-2-(3-hydroxyphenyl)-8-oxo-7-hydropurine-6-carboxamide;
9-(2-Chlorophenyl)-8-oxo-2-(3-pyridyl)-7-hydropurine-6-carboxamide;
8-oxo-2-(3-pyridyl)-9-[2-(trifluoromethyl)phenyl]-7-hydropurine-6-carboxamide;
9-(3-Chloro-2-fluorophenyl)-2-(3-hydroxyphenyl)-8-oxo-7-hydropurine-6-carboxamide;
9-(2-Fluoro-3-trifluoromethylphenyl)-2-(3-hydroxyphenyl)-8-oxo-7-hydropurine-6-carboxamide;
9-(2, 3, 4-Trifluorophenyl)-2-(3-hydroxyphenyl)-8-oxo-7-hydropurine-6-carboxamide;
2-(1H-Benzo[d]imidazol-6-yl)-9-(2-methoxyphenyl)-8-oxo-8,9-dihydro-7H-purine-6-carboxamide;
2-[3-(Acetylamino)phenyl]-9-(2-methoxyphenyl)-8-oxo-7-hydropurine-6-carboxamide;
2-(3-hydroxyphenyl)-8-(2-methoxyphenyl)-6-oxo-5,6,7,8-tetrahydropteridine-4-carbox-amide;
9-(2-Methoxyphenyl)-8-oxo-2-pyrazol-4-yl-7-hydropurine-6-carboxamide;
9-(2-Methoxyphenyl)-8-oxo-2-pyrazol-3-yl-7-hydropurine-6-carboxamide;
9-(4-Aminocyclohexyl)-2-(3-hydroxyphenyl)-8-oxo-7-hydropurine-6-carboxamide;
2-[3-(Difluoromethyl)phenyl]-9-(2-methoxyphenyl)-8-oxo-7-hydropurine-6-carbox-amide;
2-[5-(Difluoromethyl)-2-fluorophenyl]-9-(2-methoxyphenyl)-8-oxo-7-hydropurine-6-carboxamide;
2-(1H-benzo[d]imidazol-4-yl)-9-(2-methoxyphenyl)-8-oxo-8,9-dihydro-7H-purine-6-carboxamide;
2-(6-Hydroxypyridin-3-yl)-8-oxo-9-(2-(trifluoromethyl)phenyl)-8,9-dihydro-7H-purine-6-carboxamide;
2-(1H-benzo[d]imidazol-6-yl)-9-(2-fluorophenyl)-8-oxo-8,9-dihydro-7H-purine-6-carboxamide;
2-Benzimidazol-6-yl-8-oxo-9-[2-(trifluoromethyl)phenyl]-7-hydropurine-6-carboxamide;
2-(5-Chloropyridin-3-yl)-8-oxo-9-(2-(trifluoromethyl)phenyl)-8,9-dihydro-7H-purine-6-carboxamide;
trans-4-(6-Carbamoyl-9-(2-methoxyphenyl)-8-oxo-8,9-dihydro-7H-purin-2-ylamino) cyclohexyl carbamate;
(R)-9-(2-Methoxyphenyl)-8-oxo-2-(pyrrolidin-3-ylamino)-8,9-dihydro-7H-purine-6-carboxamide;
(S)-9-(2-Methoxyphenyl)-8-oxo-2-(pyrrolidin-3-ylamino)-8,9-dihydro-7H-purine-6-carboxamide;
(cis)-4-(6-Carbamoyl-9-(2-methoxyphenyl)-8-oxo-8,9-dihydro-7H-purin-2-ylamino) cyclohexyl carbamate;
2-(trans-4-Hydroxycyclohexylamino)-9-(2-methoxyphenyl)-8-oxo-8,9-dihydro-7H-purine-6-carboxamide;
2-(4-Chloropyridin-3-yl)-8-oxo-9-(2-(trifluoromethyl)phenyl)-8,9-dihydro-7H-purine-6-carboxamide;
2-(cis-4-Hydroxycyclohexylamino)-9-(2-methoxyphenyl)-8-oxo-8,9-dihydro-7H-purine-6-carboxamide;
2-(4-((1H-Imidazol-1-yl)methyl)phenylamino)-9-(2-methoxyphenyl)-8-oxo-8,9-dihydro-7H-purine-6-carboxamide;
2-(4-Hydroxypyridin-3-yl)-8-oxo-9-(2-(trifluoromethyl)phenyl)-8,9-dihydro-7H-purine-6-carboxamide;
(R)-9-(2-Methoxyphenyl)-8-oxo-2-(pyrrolidin-2-ylmethylamino)-8,9-dihydro-7H-purine-6-carboxamide;
(S)-9-(2-Methoxyphenyl)-8-oxo-2-(pyrrolidin-2-ylmethylamino)-8,9-dihydro-7H-purine-6-carboxamide;
2-(4-(1H-1,2,4-Triazol-3-yl)phenyl)-9-(2-methoxyphenyl)-8-oxo-7-hydropurine-6-carboxamide;
2-(2-Hydroxyethylamino)-9-(2-methoxyphenyl)-8-oxo-8,9-dihydro-7H-purine-6-carboxamide;
9-(2-Methoxyphenyl)-8-oxo-2-(2-(trifluoromethyl)-1H-benzo[d]imidazol-6-yl)-8,9-dihydro-7H-purine-6-carboxamide;
2-(3-(1H-1,2,4-Triazol-3-yl)phenyl)-9-(2-methoxyphenyl)-8-oxo-7-hydropurine-6-carboxamide;
9-(Biphenyl-2-yl)-2-(3-hydroxyphenyl)-8-oxo-8,9-dihydro-7H-purine-6-carboxamide;
2-(4-(1H-1,2,4-Triazol-3-yl)phenyl)-9-(2-fluorophenyl)-8-oxo-7-hydropurine-6-carboxamide;
2-(4-(1H-1,2,4-Triazol-3-yl)phenyl)-9-(2-isopropylphenyl)-8-oxo-8,9-dihydro-7H-purine-6-carboxamide;
9-(2-Methoxyphenyl)-2-(2-methyl-1H-benzo[d]imidazol-6-yl)-8-oxo-8,9-dihydro-7H-purine-6-carboxamide;
2-(3-(Hydroxymethyl)phenylamino)-9-(2-methoxyphenyl)-8-oxo-8,9-dihydro-7H-purine-6-carboxamide;
2-(2-(Hydroxymethyl)phenylamino)-9-(2-methoxyphenyl)-8-oxo-8,9-dihydro-7H-purine-6-carboxamide;
9-(2-tert-Butylphenyl)-2-(3-hydroxyphenyl)-8-oxo-8,9-dihydro-7H-purine-6-carboxamide;
2-(3-Hydroxyphenyl)-8-oxo-9-(2-phenoxyphenyl)-8,9-dihydro-7H-purine-6-carboxamide;
2-(1H-Benzo[d]imidazol-6-yl)-9-(2-isopropylphenyl)-8-oxo-8,9-dihydro-7H-purine-6-carboxamide;
2-(1H-Indazol-4-yl)-9-(2-methoxyphenyl)-8-oxo-8,9-dihydro-7H-purine-6-carboxamide;
2-(2-Hydroxypyridin-3-yl)-8-oxo-9-(2-(trifluoromethyl)phenyl)-8,9-dihydro-7H-purine-6-carboxamide;
2-(1H-Imidazo[4,5-b]pyridin-6-yl)-9-(2-methoxyphenyl)-8-oxo-8,9-dihydro-7H-purine-6-carboxamide;
2-(4-(1H-Imidazol-1-yl)phenyl)-9-(2-isopropylphenyl)-8-oxo-8,9-dihydro-7H-purine-6-carboxamide;
9-(2-Cyclohexylphenyl)-2-(3-hydroxyphenyl)-8-oxo-8,9-dihydro-7H-purine-6-carboxamide;
2-(4-(1H-Imidazol-2-yl)phenyl)-9-(2-isopropylphenyl)-8-oxo-8,9-dihydro-7H-purine-6-carboxamide;
2-(1H-Benzo[d]imidazol-1-yl)-9-(2-methoxyphenyl)-8-oxo-8,9-dihydro-7H-purine-6-carboxamide;
2-(1H-Imidazo[4,5-b]pyridin-6-yl)-9-(2-isopropylphenyl)-8-oxo-8,9-dihydro-7H-purine-6-carboxamide;
9-(2-Isopropylphenyl)-8-oxo-2-(1H-pyrrolo[2,3-b]pyridin-5-yl)-8,9-dihydro-7H-purine-6-carboxamide;
2-(1H-Imidazo[4,5-b]pyridin-6-yl)-8-oxo-9-(2-(trifluoromethyl)phenyl)-8,9-dihydro-7H-purine-6-carboxamide;
9-(2-Methoxyphenyl)-2-(2-(methylthio)-1H-benzo[d]imidazol-5-yl)-8-oxo-8,9-dihydro-7H-purine-6-carboxamide;
2-(1H-Indol-5-yl)-9-(2-isopropylphenyl)-8-oxo-8,9-dihydro-7H-purine-6-carboxamide;
9-(Cyclohexylmethyl)-2-(3-hydroxyphenyl)-8-oxo-8,9-dihydro-7H-purine-6-carboxamide;
9-(2,3-Dihydro-1H-inden-1-yl)-2-(3-hydroxyphenyl)-8-oxo-8,9-dihydro-7H-purine-6-carboxamide;
2-(3-Hydroxyphenyl)-9-isobutyl-8-oxo-8,9-dihydro-7H-purine-6-carboxamide;
9-(trans-4-Methoxycyclohexyl)-2-(3-hydroxyphenyl)-8-oxo-8,9-dihydro-7H-purine-6-carboxamide;
9-(cis-4-Methoxycyclohexyl)-2-(3-hydroxyphenyl)-8-oxo-8,9-dihydro-7H-purine-6-carboxamide;
2-(3-Hydroxyphenyl)-8-oxo-9-(5,6,7,8-tetrahydronaphthalen-1-yl)-8,9-dihydro-7H-purine-6-carboxamide;
2-(4-(1H-1,2,4-Triazol-3-yl)phenyl)-9-cyclohexyl-8-oxo-8,9-dihydro-7H-purine-6-carboxamide;
2-(3-Hydroxyphenyl)-9-(1H-indol-4-yl)-8-oxo-8,9-dihydro-7H-purine-6-carboxamide;
9-(2-Fluoro-3-methoxyphenyl)-2-(3-hydroxyphenyl)-8-oxo-8,9-dihydro-7H-purine-6-carboxamide;
9-(2-Fluoro-5-methoxyphenyl)-2-(3-hydroxyphenyl)-8-oxo-8,9-dihydro-7H-purine-6-carboxamide;
9-Cyclohexyl-2-(1H-imidazo[4,5-b]pyridin-6-yl)-8-oxo-8,9-dihydro-7H-purine-6-carboxamide;
2-(3-Hydroxyphenyl)-8-oxo-9-(tetrahydro-2H-pyran-4-yl)-8,9-dihydro-7H-purine-6-carboxamide;
2-(3-Hydroxyphenyl)-8-oxo-9-((tetrahydro-2H-pyran-4-yl)methyl)-8,9-dihydro-7H-purine-6-carboxamide;
9-(2-Cyclopentylphenyl)-2-(3-hydroxyphenyl)-8-oxo-8,9-dihydro-7H-purine-6-carboxamide;
2-(3-Hydroxyphenyl)-8-oxo-9-(piperidin-4-yl)-8,9-dihydro-7H-purine-6-carboxamide;
9-(2-Fluoro-4-methoxyphenyl)-2-(3-hydroxyphenyl)-8-oxo-8,9-dihydro-7H-purine-6-carboxamide;
2-(1H-benzo[d]imidazol-6-yl)-9-cyclohexyl-8-oxo-8,9-dihydro-7H-purine-6-carboxamide;
2-Benzimidazol-6-yl-9-(trans-4-methoxycyclohexyl)-8-oxo-7-hydropurine-6-carboxamide;
2-(4-(Aminomethyl)phenyl)-9-(2-methoxyphenyl)-8-oxo-8,9-dihydro-7H-purine-6-carboxamide;
2-(3-Hydroxyphenyl)-9-(cis-4-(methoxymethyl)cyclohexyl)-8-oxo-8,9-dihydro-7H-purine-6-carboxamide;
9-(trans-4-Aminocyclohexyl)-2-(3-hydroxyphenyl)-8-oxo-8,9-dihydro-7H-purine-6-carboxamide;
2-(3-Hydroxyphenyl)-9-(2-isobutylphenyl)-8-oxo-8,9-dihydro-7H-purine-6-carboxamide;
(R)-2-(3-Hydroxyphenyl)-8-oxo-9-(tetrahydrofuran-3-yl)-8,9-dihydro-7H-purine-6-carboxamide;
(S)-2-(3-Hydroxyphenyl)-8-oxo-9-(tetrahydrofuran-3-yl)-8,9-dihydro-7H-purine-6-carboxamide;
2-(3-(Aminomethyl)phenyl)-9-(2-methoxyphenyl)-8-oxo-8,9-dihydro-7H-purine-6-carboxamide;
2-(4-(1H-1,2,3-Triazol-5-yl)phenyl)-9-(2-isopropylphenyl)-8-oxo-8,9-dihydro-7H-purine-6-carboxamide;
2-(4-(1H-1,2,4-Triazol-3-yl)phenyl)-9-(cis-4-methoxycyclohexyl)-8-oxo-8,9-dihydro-7H-purine-6-carboxamide;
2-(1H-Benzo[d]imidazol-6-yl)-9-(cis-4-methoxycyclohexyl)-8-oxo-8,9-dihydro-7H-purine-6-carboxamide;
2-(1H-Imidazo[4,5-b]pyridin-6-yl)-9-(cis-4-methoxycyclohexyl)-8-oxo-8,9-dihydro-7H-purine-6-carboxamide;
2-(3-Hydroxyphenyl)-9-((1r,4r)-4-(methoxymethyl)cyclohexyl)-8-oxo-8,9-dihydro-7H-purine-6-carboxamide; and
9-(2-Isopropylphenyl)-2-(4-(5-methyl-4H-1,2,4-triazol-3-yl)phenyl)-8-oxo-8,9-dihydro-7H-purine-6-carboxamide,
   and pharmaceutically acceptable salts, clathrates, solvates, stereoisomers, tautomers, and prodrugs thereof.

In one embodiment, the TOR kinase inhibitors include compounds having the following formula (III):

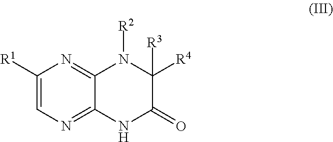

and pharmaceutically acceptable salts, clathrates, solvates, stereoisomers, tautomers, and prodrugs thereof, wherein:

R¹ is substituted or unsubstituted C₁₋₈ alkyl, substituted or unsubstituted aryl, substituted or unsubstituted cycloalkyl, substituted or unsubstituted heterocyclyl, or substituted or unsubstituted heterocyclylalkyl;

R² is H, substituted or unsubstituted C₁₋₈ alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl;

R³ and R⁴ are each independently H, substituted or unsubstituted C₁₋₈ alkyl, substituted or unsubstituted aryl, substituted or unsubstituted cycloalkyl, substituted or unsubstituted heterocyclyl, substituted or unsubstituted heterocyclylalkyl, substituted or unsubstituted aralkyl, substituted or unsubstituted cycloalkylalkyl, or R³ and R⁴, together with the atoms to which they are attached, form a substituted or unsubstituted cycloalkyl or substituted or unsubstituted heterocyclyl;

or R² and one of R³ and R⁴, together with the atoms to which they are attached, form a substituted or unsubstituted heterocyclyl,

wherein in certain embodiments, the TOR kinase inhibitors do not include the compounds depicted below, namely:

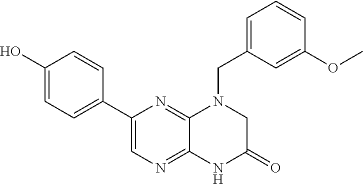 [SMILES: COc1cccc(CN2CC(=O)Nc3ncc(-c4ccc(O)cc4)nc32)c1]

6-(4-hydroxyphenyl)-4-(3-methoxybenzyl)-3,4-dihydropyrazino[2,3-b]pyrazin-2(1H)-one;

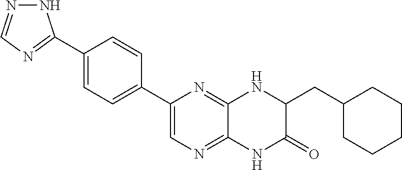 [SMILES: O=C1Nc2ncc(-c3ccc(-c4ncn[nH]4)cc3)nc2NC1CC1CCCCC1]

6-(4-hydroxyphenyl)-4-(3-methoxybenzyl)-3,4-dihydropyrazino[2,3-b]pyrazin-2(1H)-one;
   or,

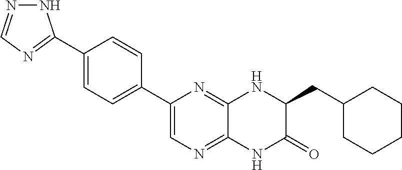 [SMILES: O=C1Nc2ncc(-c3ccc(-c4ncn[nH]4)cc3)nc2N[C@H]1CC1CCCCC1]

(R)-6-(4-(1H-1,2,4-triazol-5-yl)phenyl)-3-(cyclohexylmethyl)-3,4-dihydropyrazino[2,3-b]pyrazin-2(1H)-one.

In some embodiments of compounds of formula (III), R¹ is substituted or unsubstituted aryl or substituted or unsubstituted heteroaryl. In one embodiment, R¹ is phenyl, pyridyl, pyrimidyl, benzimidazolyl, indolyl, indazolyl, 1H-pyrrolo[2,3-b]pyridyl, 1H-imidazo[4,5-b]pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl (for example, methyl), substituted or unsubstituted heterocyclyl (for example, substituted or unsubstituted triazolyl or pyrazolyl), halogen (for example, fluorine), aminocarbonyl, cyano, hydroxyalkyl (for example, hydroxypropyl), and hydroxy. In other embodiments, R¹ is pyridyl substituted with one or more substituents independently selected from the group consisting of substituted or unsubstituted C₁₋₈ alkyl, substituted or unsubstituted heterocyclyl (for example, substituted or unsubstituted triazolyl), halogen, aminocarbonyl, cyano, hydroxyalkyl, —OR, and —NR₂, wherein each R is independently H, or a substituted or unsubstituted C₁₋₄ alkyl. In yet other embodiments, R¹ is 1H-pyrrolo[2,3-b]pyridyl or benzimidazolyl, each optionally substituted with one or more substituents independently selected from the group consisting of substituted or unsubstituted C₁₋₈ alkyl, and —NR₂, wherein each R is independently H, or a substituted or unsubstituted C₁₋₄ alkyl.

In some embodiments of compounds of formula (III), R¹ is

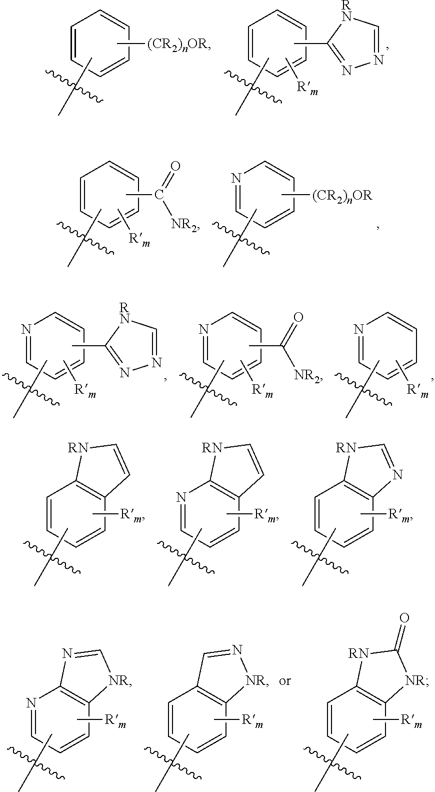

wherein R is at each occurrence independently H, or a substituted or unsubstituted C₁₋₄ alkyl (for example, methyl); R′ is at each occurrence independently a substituted or unsubstituted C₁₋₄ alkyl, halogen (for example, fluorine), cyano, —OR, or —NR₂; m is 0-3; and n is 0-3. It will be understood by those skilled in the art that any of the substitutents R′ may be attached to any suitable atom of any of the rings in the fused ring systems. It will also be understood by those skilled in the art that the connecting bond of R¹ (designated by the bisecting wavy line) may be attached to any of the atoms in any of the rings in the fused ring systems.

In some embodiments of compounds of formula (III), R¹ is

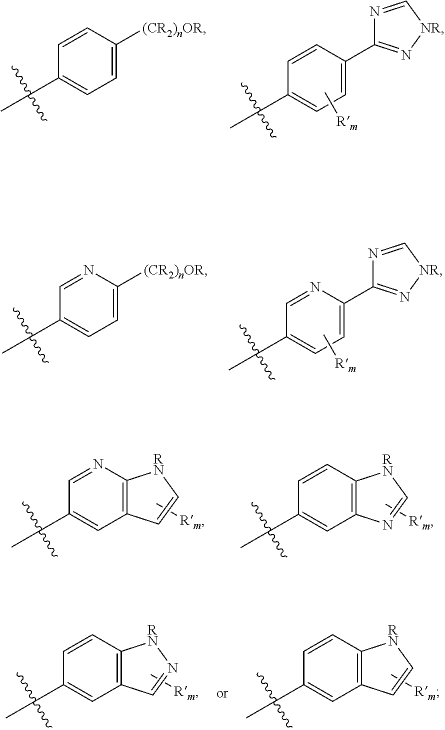

wherein R is at each occurrence independently H, or a substituted or unsubstituted C₁₋₄ alkyl; R′ is at each occurrence independently a substituted or unsubstituted C₁₋₄ alkyl, halogen, cyano, —OR, or —NR₂; m is 0-3; and n is 0-3.

In some embodiments of compounds of formula (III), R² is H, substituted or unsubstituted C₁₋₈ alkyl, substituted or unsubstituted cycloalkyl, substituted or unsubstituted heterocyclyl, substituted or unsubstituted C₁₋₄ alkyl-heterocyclyl, substituted or unsubstituted C₁₋₄ alkyl-aryl, or substituted or unsubstituted C₁₋₄ alkyl-cycloalkyl. For example, R² is H, methyl, ethyl, n-propyl, isopropyl, n-butyl, sec-butyl, isobutyl, tert-butyl, n-pentyl, isopentyl, cyclopentyl, cyclohexyl, tetrahydrofuranyl, tetrahydropyranyl, (C₁₋₄ alkyl)-phenyl, (C₁₋₄ alkyl)-cyclopropyl, (C₁₋₄ alkyl)-cyclobutyl, (C₁₋₄ alkyl)-cyclopentyl, (C₁₋₄ alkyl)-cyclohexyl, (C₁₋₄ alkyl)-pyrrolidyl, (C₁₋₄ alkyl)-piperidyl, (C₁₋₄ alkyl)-piperazinyl, (C₁₋₄ alkyl)-morpholinyl, (C₁₋₄ alkyl)-tetrahydrofuranyl, or (C₁₋₄ alkyl)-tetrahydropyranyl, each optionally substituted.

In other embodiments, R² is H, C₁₋₄ alkyl, (C₁₋₄alkyl)(OR),

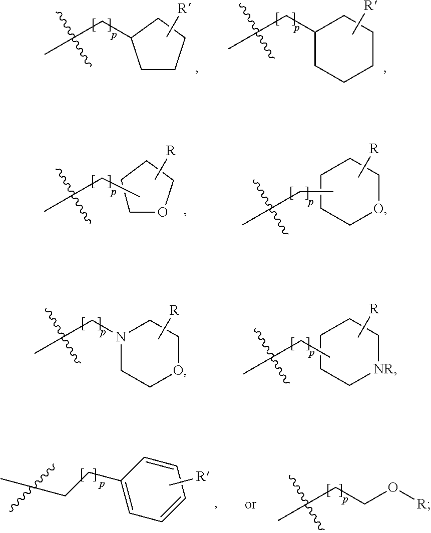

wherein R is at each occurrence independently H, or a substituted or unsubstituted C₁₋₄ alkyl (for example, methyl); R′ is at each occurrence independently H, —OR, cyano, or a substituted or unsubstituted C₁₋₄ alkyl (for example, methyl); and p is 0-3.

In some such embodiments, R² is H, C₁₋₄ alkyl, (C₁₋₄ alkyl)(OR),

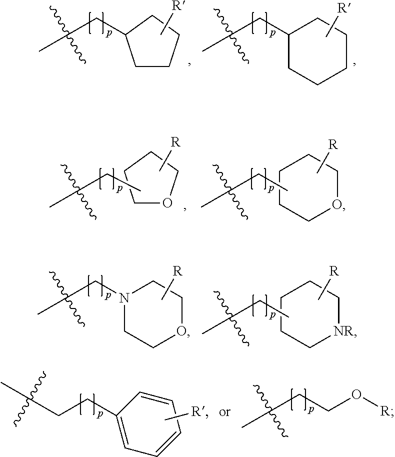

wherein R is at each occurrence independently H, or a substituted or unsubstituted C₁₋₂ alkyl; R′ is at each occurrence independently H, —OR, cyano, or a substituted or unsubstituted C₁₋₂ alkyl; and p is 0-1.

In some other embodiments of compounds of formula (III), R² and one of R³ and R⁴ together with the atoms to which they are attached form a substituted or unsubstituted heterocyclyl. For example, in some embodiments, the compound of formula (III) is

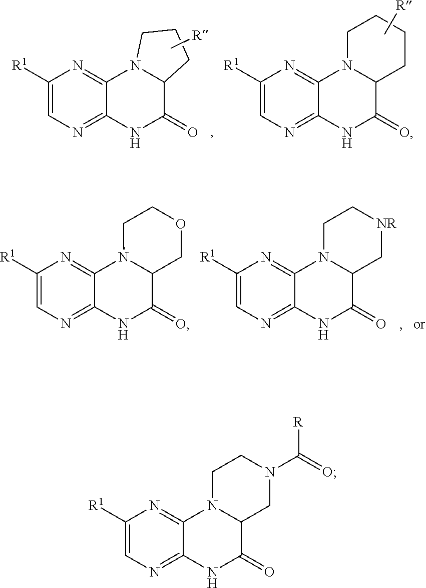

wherein R is at each occurrence independently H, or a substituted or unsubstituted C₁₋₄ alkyl; R″ is H, OR, or a substituted or unsubstituted C₁₋₄ alkyl; and R¹ is as defined herein.

In some embodiments of compounds of formula (III), R³ and R⁴ are both H. In others, one of R³ and R⁴ is H and the other is other than H. In still others, one of R³ and R⁴ is C₁₋₄ alkyl (for example, methyl) and the other is H. In still others, both of R³ and R⁴ are C₁₋₄ alkyl (for example, methyl).

In some such embodiments described above, R¹ is substituted or unsubstituted aryl, or substituted or unsubstituted heteroaryl. For example, R¹ is phenyl, pyridyl, pyrimidyl, benzimidazolyl, indolyl, indazolyl, 1H-pyrrolo[2,3-b]pyridyl, 1H-imidazo[4,5-b]pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl, substituted or unsubstituted heterocyclyl, halogen, aminocarbonyl, cyano, hydroxyalkyl and hydroxy. In others, R¹ is pyridyl substituted with one or more substituents independently selected from the group consisting of cyano, substituted or unsubstituted C₁₋₈ alkyl, substituted or unsubstituted heterocyclyl, hydroxyalkyl, halogen, aminocarbonyl, —OR, and —NR₂, wherein each R is independently H, or a substituted or unsubstituted C₁₋₄ alkyl. In others, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In certain embodiments, the compounds of formula (III) have an R¹ group set forth herein and an R² group set forth herein.

In some embodiments of compounds of formula (III), the compound at a concentration of 10 μM inhibits mTOR, DNA-PK, or PI3K or a combination thereof, by at least about 50%. Compounds of formula (III) may be shown to be inhibitors of the kinases above in any suitable assay system.

Representative TOR kinase inhibitors of formula (III) include:
6-(1H-pyrrolo[2,3-b]pyridin-3-yl)-4-(2-(tetrahydro-2H-pyran-4-yl)ethyl)-3,4-dihydropyrazino[2,3-b]pyrazin-2(1H)-one;
6-(4-methyl-6-(1H-1,2,4-triazol-3-yl)pyridin-3-yl)-4-((tetrahydro-2H-pyran-4-yl)methyl)-3,4-dihydropyrazino[2,3-b]pyrazin-2(1H)-one;
6-(5-fluoro-2-methyl-4-(1H-1,2,4-triazol-3-yl)phenyl)-4-((trans-4-methoxycyclohexyl)methyl)-3,4-dihydropyrazino[2,3-b]pyrazin-2(1H)-one;
6-(5-fluoro-2-methyl-4-(1H-1,2,4-triazol-3-yl)phenyl)-4-((cis-4-methoxycyclohexyl)methyl)-3,4-dihydropyrazino[2,3-b]pyrazin-2(1H)-one;
6-(6-(1H-1,2,4-triazol-3-yl)pyridin-3-yl)-4-((trans-4-methoxycyclohexyl)methyl)-3,4-dihydropyrazino[2,3-b]pyrazin-2(1H)-one;
6-(5-fluoro-2-methyl-4-(1H-1,2,4-triazol-3-yl)phenyl)-4-((trans-4-hydroxycyclohexyl)methyl)-3,4-dihydropyrazino[2,3-b]pyrazin-2(1H)-one;
6-(6-(1H-1,2,4-triazol-3-yl)pyridin-3-yl)-4-((cis-4-methoxycyclohexyl)methyl)-3,4-dihydropyrazino [2,3-b]pyrazin-2(1H)-one;
6-(6-(1H-1,2,4-triazol-3-yl)pyridin-3-yl)-4-((trans-4-hydroxycyclohexyl)methyl)-3,4-dihydropyrazino [2,3-b]pyrazin-2(1H)-one;
6-(6-(1H-1,2,4-triazol-3-yl)pyridin-3-yl)-4-(cis-4-hydroxycyclohexyl)-3,4-dihydropyrazino[2,3-b]pyrazin-2(1H)-one;
6-(6-(1H-1,2,4-triazol-3-yl)pyridin-3-yl)-4-((cis-4-hydroxycyclohexyl)methyl)-3,4-dihydropyrazino [2,3-b]pyrazin-2(1H)-one;
6-(5-fluoro-2-methyl-4-(1H-1,2,4-triazol-3-yl)phenyl)-4-(trans-4-methoxycyclohexyl)-3,4-dihydropyrazino [2,3-b]pyrazin-2(1H)-one;
6-(6-(1H-1,2,4-triazol-3-yl)pyridin-3-yl)-4-(trans-4-methoxycyclohexyl)-3,4-dihydropyrazino [2,3-b]pyrazin-2(1H)-one;
6-(6-(1H-1,2,4-triazol-3-yl)pyridin-3-yl)-4-(trans-4-hydroxycyclohexyl)-3,4-dihydropyrazino [2,3-b]pyrazin-2(1H)-one;
6-(5-fluoro-2-methyl-4-(1H-1,2,4-triazol-3-yl)phenyl)-4-((cis-4-hydroxycyclohexyl)methyl)-3,4-dihydropyrazino [2,3-b]pyrazin-2(1H)-one;
6-(6-(1H-1,2,4-triazol-3-yl)pyridin-3-yl)-4-(cis-4-methoxycyclohexyl)-3,4-dihydropyrazino[2,3-b]pyrazin-2(1H)-one;
6-(6-(1H-1,2,4-triazol-3-yl)pyridin-3-yl)-4-(2-methoxyethyl)-3,4-dihydropyrazino[2,3-b]pyrazin-2(1H)-one;
6-(6-(1H-1,2,4-triazol-3-yl)pyridin-3-yl)-4-isopropyl-3,4-dihydropyrazino[2,3-b]pyrazin-2(1H)-one;
6-(5-fluoro-2-methyl-4-(1H-1,2,4-triazol-3-yl)phenyl)-4-(cis-4-hydroxycyclohexyl)-3,4-dihydropyrazino [2,3-b]pyrazin-2(1H)-one;
6-(5-fluoro-2-methyl-4-(1H-1,2,4-triazol-3-yl)phenyl)-4-(cis-4-methoxycyclohexyl)-3,4-dihydropyrazino [2,3-b]pyrazin-2(1H)-one;
6-(5-fluoro-2-methyl-4-(1H-1,2,4-triazol-3-yl)phenyl)-4-(2-methoxyethyl)-3,4-dihydropyrazino [2,3-b]pyrazin-2(1H)-one;
6-(6-(1H-1,2,4-triazol-3-yl)pyridin-3-yl)-4-(tetrahydro-2H-pyran-4-yl)-3,4-dihydropyrazino[2,3-b]pyrazin-2(1H)-one;
6-(6-(1H-1,2,4-triazol-3-yl)pyridin-3-yl)-4-ethyl-3,4-dihydropyrazino[2,3-b]pyrazin-2(1H)-one;
6-(5-fluoro-2-methyl-4-(1H-1,2,4-triazol-3-yl)phenyl)-4-(trans-4-hydroxycyclohexyl)-3,4-dihydropyrazino [2,3-b]pyrazin-2(1H)-one;
6-(5-fluoro-2-methyl-4-(1H-1,2,4-triazol-3-yl)phenyl)-4-(tetrahydro-2H-pyran-4-yl)-3,4-dihydropyrazino [2,3-b]pyrazin-2(1H)-one;
6-(5-fluoro-2-methyl-4-(1H-1,2,4-triazol-3-yl)phenyl)-4-isopropyl-3,4-dihydropyrazino[2,3-b]pyrazin-2(1H)-one;
4-ethyl-6-(5-fluoro-2-methyl-4-(1H-1,2,4-triazol-3-yl)phenyl)-3,4-dihydropyrazino[2,3-b]pyrazin-2(1H)-one;
6-(3-fluoro-2-methyl-4-(1H-1,2,4-triazol-3-yl)phenyl)-4-(tetrahydro-2H-pyran-4-yl)-3,4-dihydropyrazino [2,3-b]pyrazin-2(1H)-one;
6-(3-fluoro-2-methyl-4-(1H-1,2,4-triazol-3-yl)phenyl)-4-(cis-4-methoxycyclohexyl)-3,4-dihydropyrazino [2,3-b]pyrazin-2(1H)-one;
6-(3-fluoro-2-methyl-4-(1H-1,2,4-triazol-3-yl)phenyl)-4-(trans-4-methoxycyclohexyl)-3,4-dihydropyrazino [2,3-b]pyrazin-2(1H)-one;
4-(2-methoxyethyl)-6-(4-methyl-6(1H-1,2,4-triazol-3-yl)pyridin-3-yl)-3,4-dihydropyrazino[2,3-b]pyrazin-2(1H)-one;
6-(3-(1H-1,2,4-triazol-5-yl)phenyl)-4-(2-(tetrahydro-2H-pyran-4-yl)ethyl)-3,4-dihydropyrazino [2,3-b]pyrazin-2(1H)-one;
5-(8-(2-methoxyethyl)-6-oxo-5,6,7,8-tetrahydropyrazino[2,3-b]pyrazin-2-yl)-4-methylpicolinamide;
3-(6-oxo-8-(2-(tetrahydro-2H-pyran-4-yl)ethyl)-5,6,7,8-tetrahydropyrazino[2,3-b]pyrazin-2-yl)benzamide;
3-(6-oxo-8-(2-(tetrahydro-2H-pyran-4-yl)ethyl)-5,6,7,8-tetrahydropyrazino[2,3-b]pyrazin-2-yl)benzonitrile;
5-(8-(trans-4-methoxycyclohexyl)-6-oxo-5,6,7,8-tetrahydropyrazino[2,3-b]pyrazin-2-yl)-4-methylpicolinamide;
6-(1H-imidazo[4,5-b]pyridin-6-yl)-4-(2-(tetrahydro-2H-pyran-4-yl)ethyl)-3,4-dihydropyrazino[2,3-b]pyrazin-2(1H)-one;
6-(1H-indazol-6-yl)-4-(2-(tetrahydro-2H-pyran-4-yl)ethyl)-3,4-dihydropyrazino[2,3-b]pyrazin-2(1H)-one;
4-((1R,3S)-3-methoxycyclopentyl)-6-(2-methyl-6-(4H-1,2,4-triazol-3-yl)pyridin-3-yl)-3,4-dihydropyrazino[2,3-b]pyrazin-2(1H)-one;
4-((1S,3R)-3-methoxycyclopentyl)-6-(2-methyl-6-(4H-1,2,4-triazol-3-yl)pyridin-3-yl)-3,4-dihydropyrazino[2,3-b]pyrazin-2(1H)-one;
4-((1R,3R)-3-methoxycyclopentyl)-6-(2-methyl-6-(4H-1,2,4-triazol-3-yl)pyridin-3-yl)-3,4-dihydropyrazino[2,3-b]pyrazin-2(1H)-one;
4-((1S,3S)-3-methoxycyclopentyl)-6-(2-methyl-6-(4H-1,2,4-triazol-3-yl)pyridin-3-yl)-3,4-dihydropyrazino[2,3-b]pyrazin-2(1H)-one;
4-ethyl-6-(2-methyl-6-(4H-1,2,4-triazol-3-yl)pyridin-3-yl)-3,4-dihydropyrazino[2,3-b]pyrazin-2(1H)-one;
6-(1H-pyrrolo[2,3-b]pyridin-5-yl)-4-(2-(tetrahydro-2H-pyran-4-yl)ethyl)-3,4-dihydropyrazino[2,3-b]pyrazin-2(1H)-one;
6-(1H-indol-6-yl)-4-(2-(tetrahydro-2H-pyran-4-yl)ethyl)-3,4-dihydropyrazino[2,3-b]pyrazin-2(1H)-one;
6-(1H-indol-5-yl)-4-(2-(tetrahydro-2H-pyran-4-yl)ethyl)-3,4-dihydropyrazino[2,3-b]pyrazin-2(1H)-one;
4-(((1R,3S)-3-methoxycyclopentypmethyl)-6-(2-methyl-6-(4H-1,2,4-triazol-3-yl)pyridin-3-yl)-3,4-dihydropyrazino[2,3-b]pyrazin-2(1H)-one;
4-(((1S,3R)-3-methoxycyclopentyl)methyl)-6-(2-methyl-6-(4H-1,2,4-triazol-3-yl)pyridin-3-yl)-3,4-dihydropyrazino[2,3-b]pyrazin-2(1H)-one;
6-(3-fluoro-2-methyl-4-(4H-1,2,4-triazol-3-yl)phenyl)-4-(2-(tetrahydro-2H-pyran-4-yl)ethyl)-3,4-dihydropyrazino[2,3-b]pyrazin-2(1H)-one;
6-(3-fluoro-2-methyl-4-(4H-1,2,4-triazol-3-yl)phenyl)-4-(2-methoxyethyl)-3,4-dihydropyrazino[2,3-b]pyrazin-2(1H)-one;
3,3-dimethyl-6-(4-methyl-6-(4H-1,2,4-triazol-3-yl)pyridin-3-yl)-4-((tetrahydro-2H-pyran-4-yl)methyl)-3,4-dihydropyrazino[2,3-b]pyrazin-2(1H)-one;
6-(6-(2-hydroxypropan-2-yl)pyridin-3-yl)-4-((1R,3S)-3-methoxycyclopentyl)-3,4-dihydropyrazino[2,3-b]pyrazin-2(1H)-one;
6-(6-(2-hydroxypropan-2-yl)pyridin-3-yl)-4-((1S,3R)-3-methoxycyclopentyl)-3,4-dihydropyrazino[2,3-b]pyrazin-2(1H)-one;
6-(6-(2-hydroxypropan-2-yl)pyridin-3-yl)-4-((1S,3S)-3-methoxycyclopentyl)methyl)-3,4-dihydropyrazino[2,3-b]pyrazin-2(1H)-one;
6-(6-(2-hydroxypropan-2-yl)pyridin-3-yl)-4-((1R,3R)-3-methoxycyclopentyl)methyl)-3,4-dihydropyrazino[2,3-b]pyrazin-2(1H)-one;
6-(6-(2-hydroxypropan-2-yl)pyridin-3-yl)-4-((1S,3S)-3-methoxycyclopentyl)-3,4-dihydropyrazino[2,3-b]pyrazin-2(1H)-one;
6-(6-(2-hydroxypropan-2-yl)pyridin-3-yl)-4-((1R,3R)-3-methoxycyclopentyl)-3,4-dihydropyrazino[2,3-b]pyrazin-2(1H)-one;
6-(6-(2-hydroxypropan-2-yl)pyridin-3-yl)-4-((1R,3S)-3-methoxycyclopentyl)methyl)-3,4-dihydropyrazino[2,3-b]pyrazin-2(1H)-one;
6-(6-(2-hydroxypropan-2-yl)pyridin-3-yl)-4-((1S,3R)-3-methoxycyclopentyl)methyl)-3,4-dihydropyrazino[2,3-b]pyrazin-2(1H)-one;
6-(3-fluoro-4-(4H-1,2,4-triazol-3-yl)phenyl)-4-(2-methoxyethyl)-3,4-dihydropyrazino[2,3-b]pyrazin-2(1H)-one;
6-(3-fluoro-4-(4H-1,2,4-triazol-3-yl)phenyl)-4-(2-(tetrahydro-2H-pyran-4-yl)ethyl)-3,4-dihydropyrazino[2,3-b]pyrazin-2(1H)-one;
7′-(2-methyl-4-(4H-1,2,4-triazol-3-yl)phenyl)-1′-((tetrahydro-2H-pyran-4-yl)methyl)-1′H-spiro[cyclopentane-1,2′-pyrazino[2,3-b]pyrazin]-3′(4′H)-one;
7′-(2-methyl-4-(4H-1,2,4-triazol-3-yl)phenyl)-1′-((tetrahydro-2H-pyran-4-yl)methyl)-1′H-spiro[cyclobutane-1,2′-pyrazino[2,3-b]pyrazin]-3′(4′H)-one;
4-(cyclopropylmethyl)-6-(6-(2-hydroxypropan-2-yl)pyridin-3-yl)-3,4-dihydropyrazino[2,3-b]pyrazin-2(1H)-one;
7′-(2-methyl-4-(4H-1,2,4-triazol-3-yl)phenyl)-1′H-spiro[cyclopentane-1,2′-pyrazino[2,3-b]pyrazin]-3′(4′H)-one;
7′-(2-methyl-4-(4H-1,2,4-triazol-3-yl)phenyl)-1′H-spiro[cyclobutane-1,2′-pyrazino[2,3-b]pyrazin]-3′(4′H)-one;
7′-(2-methyl-4-(4H-1,2,4-triazol-3-yl)phenyl)-1′H-spiro[cyclopropane-1,2′-pyrazino[2,3-b]pyrazin]-3′(4′H)-one;
(R)-6-(4-(4H-1,2,4-triazol-3-yl)phenyl)-4-((tetrahydrofuran-2-yl)methyl)-3,4-dihydropyrazino[2,3-b]pyrazin-2(1H)-one;
(S)-6-(4-(4H-1,2,4-triazol-3-yl)phenyl)-4-((tetrahydrofuran-2-yl)methyl)-3,4-dihydropyrazino[2,3-b]pyrazin-2(1H)-one;
6-(1H-indazol-5-yl)-4-(2-(tetrahydro-2H-pyran-4-yl)ethyl)-3,4-dihydropyrazino[2,3-b]pyrazin-2(1H)-one;
4-(6-oxo-8-(2-(tetrahydro-2H-pyran-4-yl)ethyl)-5,6,7,8-tetrahydropyrazino[2,3-b]pyrazin-2-yl)benzamide;
4-(2-methoxyethyl)-3,3-dimethyl-6-(2-methyl-4-(4H-1,2,4-triazol-3-yl)phenyl)-3,4-dihydropyrazino[2,3-b]pyrazin-2(1H)-one;
4-ethyl-3,3-dimethyl-6-(2-methyl-4-(4H-1,2,4-triazol-3-yl)phenyl)-3,4-dihydropyrazino[2,3-b]pyrazin-2(1H)-one;
6-(2-methyl-4-(4H-1,2,4-triazol-3-yl)phenyl)-3,4-dihydropyrazino[2,3-b]pyrazin-2(1H)-one;
3,3-dimethyl-6-(2-methyl-6-(4H-1,2,4-triazol-3-yl)pyridin-3-yl)-4-((tetrahydro-2H-pyran-4-yl)methyl)-3,4-dihydropyrazino[2,3-b]pyrazin-2(1H)-one;
(R)-6-(6-(1-hydroxyethyl)pyridin-3-yl)-4-(2-(tetrahydro-2H-pyran-4-yl)ethyl)-3,4-dihydropyrazino[2,3-b]pyrazin-2(1H)-one;
3,3-dimethyl-6-(2-methyl-4-(4H-1,2,4-triazol-3-yl)phenyl)-4-((tetrahydro-2H-pyran-4-yl)methyl)-3,4-dihydropyrazino[2,3-b]pyrazin-2(1H)-one;
6-(6-(2-hydroxypropan-2-yl)-4-methylpyridin-3-yl)-4-(trans-4-methoxycyclohexyl)-3,4-dihydropyrazino[2,3-b]pyrazin-2(1H)-one;
6-(6-(2-hydroxypropan-2-yl)-4-methylpyridin-3-yl)-4-((tetrahydro-2H-pyran-4-yl)methyl)-3,4-dihydropyrazino[2,3-b]pyrazin-2(1H)-one;
3,3-dimethyl-6-(2-methyl-4-(4H-1,2,4-triazol-3-yl)phenyl)-3,4-dihydropyrazino[2,3-b]pyrazin-2(1H)-one;
3,3-dimethyl-6-(2-methyl-6-(4H-1,2,4-triazol-3-yl)pyridin-3-yl)-4-(2-(tetrahydro-2H-pyran-4-yl)ethyl)-3,4-dihydropyrazino[2,3-b]pyrazin-2(1H)-one;
6-(6-(2-hydroxypropan-2-yl)-2-methylpyridin-3-yl)-4-((tetrahydro-2H-pyran-4-yl)methyl)-3,4-dihydropyrazino[2,3-b]pyrazin-2(1H)-one;
6-(6-(2-hydroxypropan-2-yl)-2-methylpyridin-3-yl)-4-(trans-4-methoxycyclohexyl)-3,4-dihydropyrazino[2,3-b]pyrazin-2(1H)-one;
(S)-6-(6-(1-hydroxyethyl)pyridin-3-yl)-4-(2-(tetrahydro-2H-pyran-4-yl)ethyl)-3,4-dihydropyrazino[2,3-b]pyrazin-2(1H)-one;
3,3-dimethyl-6-(2-methyl-4-(4H-1,2,4-triazol-3-yl)phenyl)-4-(2-(tetrahydro-2H-pyran-4-yl)ethyl)-3,4-dihydropyrazino[2,3-b]pyrazin-2(1H)-one;
6-(6-(2-hydroxypropan-2-yl)pyridin-3-yl)-3,3-dimethyl-4-(2-(tetrahydro-2H-pyran-4-yl)ethyl)-3,4-dihydropyrazino[2,3-b]pyrazin-2(1H)-one;
6-(4-(2-hydroxypropan-2-yl)phenyl)-4-(trans-4-methoxycyclohexyl)-3,4-dihydropyrazino[2,3-b]pyrazin-2(1H)-one;
6-(4-(2-hydroxypropan-2-yl)phenyl)-4-((trans-4-methoxycyclohexyl)methyl)-3,4-dihydropyrazino [2,3-b]pyrazin-2(1H)-one;
4-(cis-4-methoxycyclohexyl)-6-(2-methyl-6-(4H-1,2,4-triazol-3-yl)pyridin-3-yl)-3,4-dihydropyrazino [2,3-b]pyrazin-2(1H)-one;
4-(trans-4-methoxycyclohexyl)-6-(2-methyl-6-(4H-1,2,4-triazol-3-yl)pyridin-3-yl)-3,4-dihydropyrazino [2,3-b]pyrazin-2(1H)-one;
6-(4-(2-hydroxypropan-2-yl)phenyl)-4-((tetrahydro-2H-pyran-4-yl)methyl)-3,4-dihydropyrazino [2,3-b]pyrazin-2(1H)-one;
4-(2-methoxyethyl)-6-(2-methyl-6-(4H-1,2,4-triazol-3-yl)pyridin-3-yl)-3,4-dihydropyrazino[2,3-b]pyrazin-2(1H)-one;
9-(6-(4H-1,2,4-triazol-3-yl)-3-pyridyl)-6,11,4a-trihydromorpholino[4,3-e]pyrazino[2,3-b]pyrazin-5-one;
6-(2-methyl-6-(4H-1,2,4-triazol-3-yl)pyridin-3-yl)-4-((tetrahydro-2H-pyran-4-yl)methyl)-3,4-dihydropyrazino [2,3-b]pyrazin-2(1H)-one;
5-(8-(cis-4-methoxycyclohexyl)-6-oxo-5,6,7,8-tetrahydropyrazino[2,3-b]pyrazin-2-yl)-6-methylpicolinonitrile;
6-(6-(4H-1,2,4-triazol-3-yl)pyridin-3-yl)-4-(2-(tetrahydro-2H-pyran-4-yl)ethyl)-3,4-dihydropyrazino [2,3-b]pyrazin-2(1H)-one;
9-(4-(4H-1,2,4-triazol-3-yl)-2-methylphenyl)-3-(2-methoxyacetyl)-6,11,4a-trihydropiperazino [1,2-e]pyrazino[2,3-b]pyrazin-5-one;
9-(4-(4H-1,2,4-triazol-3-yl)-2-methylphenyl)-6,11,4a-trihydropiperazino[1,2-e]pyrazino[2,3-b]pyrazin-5-one;
9-(4-(4H-1,2,4-triazol-3-yl)-2-methylphenyl)-3-(2-methoxyethyl)-6,11,4a-trihydropiperazino [1,2-e]pyrazino[2,3-b]pyrazin-5-one;
4-(cyclopentylmethyl)-6-(2-methyl-6-(4H-1,2,4-triazol-3-yl)pyridin-3-yl)-3,4-dihydropyrazino [2,3-b]pyrazin-2(1H)-one;
9-(6-(4H-1,2,4-triazol-3-yl)-2-methyl-3-pyridyl)-6,11,4a-trihydromorpholino[4,3-e]pyrazino[2,3-b]pyrazin-5-one;
4-(trans-4-hydroxycyclohexyl)-6-(6-(2-hydroxypropan-2-yl)pyridin-3-yl)-3,4-dihydropyrazino [2,3-b]pyrazin-2(1H)-one;
4-(cis-4-hydroxycyclohexyl)-6-(6-(2-hydroxypropan-2-yl)pyridin-3-yl)-3,4-dihydropyrazino[2,3-b]pyrazin-2(1H)-one;
6-(6-(2-hydroxypropan-2-yl)pyridin-3-yl)-4-((tetrahydrofuran-3-yl)methyl)-3,4-dihydropyrazino [2,3-b]pyrazin-2(1H)-one;
4-(cyclopentylmethyl)-6-(6-(2-hydroxypropan-2-yl)pyridin-3-yl)-3,4-dihydropyrazino[2,3-b]pyrazin-2(1H)-one;
6-(6-(2-hydroxypropan-2-yl)pyridin-3-yl)-4-neopentyl-3,4-dihydropyrazino[2,3-b]pyrazin-2(1H)-one;
6-(6-(2-hydroxypropan-2-yl)pyridin-3-yl)-4-isobutyl-3,4-dihydropyrazino[2,3-b]pyrazin-2(1H)-one;
3-methyl-6-(2-methyl-4-(4H-1,2,4-triazol-3-yl)phenyl)-4-(2-(tetrahydro-2H-pyran-4-yl)ethyl)-3,4-dihydropyrazino[2,3-b]pyrazin-2(1H)-one;
6-(6-(2-hydroxypropan-2-yl)pyridin-3-yl)-4-(piperidin-4-yl)-3,4-dihydropyrazino[2,3-b]pyrazin-2(1H)-one;
6-(6-(2-hydroxypropan-2-yl)pyridin-3-yl)-4-(2-(tetrahydro-2H-pyran-3-yl)ethyl)-3,4-dihydropyrazino [2,3-b]pyrazin-2(1H)-one;
8-(4-(4H-1,2,4-triazol-3-yl)-2-methylphenyl)(3aS,2R)-2-methoxy-5,10,3a-trihydropyrazino[2,3-b]pyrrolidino[1,2-e]pyrazin-4-one;
8-(4-(4H-1,2,4-triazol-3-yl)-2-methylphenyl)(2R,3aR)-2-methoxy-5,10,3a-trihydropyrazino[2,3-b]pyrrolidino[1,2-e]pyrazin-4-one;
8-(4-(4H-1,2,4-triazol-3-yl)-2-methylphenyl)(2S,3aR)-2-methoxy-5,10,3a-trihydropyrazino[2,3-b]pyrrolidino[1,2-e]pyrazin-4-one;
8-(4-(4H-1,2,4-triazol-3-yl)-2-methylphenyl)(2S,3aS)-2-methoxy-5,10,3a-trihydropyrazino[2,3-b]pyrrolidino[1,2-e]pyrazin-4-one;
6-(6-(2-hydroxypropan-2-yl)pyridin-3-yl)-4-(3-methoxypropyl)-3,4-dihydropyrazino[2,3-b]pyrazin-2(1H)-one;
(S)-6-(6-(2-hydroxypropan-2-yl)pyridin-3-yl)-4-((tetrahydrofuran-2-yl)methyl)-3,4-dihydropyrazino [2,3-b]pyrazin-2(1H)-one;
(R)-6-(6-(2-hydroxypropan-2-yl)pyridin-3-yl)-4-((tetrahydrofuran-2-yl)methyl)-3,4-dihydropyrazino [2,3-b]pyrazin-2(1H)-one;
6-(2-methyl-6-(4H-1,2,4-triazol-3-yl)pyridin-3-yl)-4-(2-(tetrahydro-2H-pyran-4-yl)ethyl)-3,4-dihydropyrazino [2,3-b]pyrazin-2(1H)-one;
9-(4-(4H-1,2,4-triazol-3-yl)-2-methylphenyl)-3-methyl-6,11,4a-trihydropiperazino[1,2-e]pyrazino[2,3-b]pyrazin-5-one;
9-(4-(4H-1,2,4-triazol-3-yl)phenyl)-6,11,4a-trihydromorpholino[4,3-e]pyrazino[2,3-b]pyrazin-5-one;
9-(4-(4H-1,2,4-triazol-3-yl)-2-methylphenyl)-6,11,4a-trihydropiperidino[1,2-e]pyrazino[2,3-b]pyrazin-5-one;
6-(6-(2-hydroxypropan-2-yl)pyridin-3-yl)-4-(trans-4-methoxycyclohexyl)-3,4-dihydropyrazino [2,3-b]pyrazin-2(1H)-one;
6-(6-(2-hydroxypropan-2-yl)pyridin-3-yl)-4-(cis-4-methoxycyclohexyl)-3,4-dihydropyrazino [2,3-b]pyrazin-2(1H)-one;
6-(6-(2-hydroxypropan-2-yl)pyridin-3-yl)-4-(2-morpholinoethyl)-3,4-dihydropyrazino[2,3-b]pyrazin-2(1H)-one;
6-(6-(2-hydroxypropan-2-yl)pyridin-3-yl)-4-phenethyl-3,4-dihydropyrazino[2,3-b]pyrazin-2(1H)-one;
6-(6-(2-hydroxypropan-2-yl)pyridin-3-yl)-4-(tetrahydro-2H-pyran-4-yl)-3,4-dihydropyrazino [2,3-b]pyrazin-2(1H)-one;
4-(cyclohexylmethyl)-6-(6-(2-hydroxypropan-2-yl)pyridin-3-yl)-3,4-dihydropyrazino[2,3-b]pyrazin-2(1H)-one;
6-(6-(2-hydroxypropan-2-yl)pyridin-3-yl)-4-((trans-4-methoxycyclohexyl)methyl)-3,4-dihydropyrazino [2,3-b]pyrazin-2(1H)-one;
6-(6-(2-hydroxypropan-2-yl)pyridin-3-yl)-4-((cis-4-methoxycyclohexyl)methyl)-3,4-dihydropyrazino [2,3-b]pyrazin-2(1H)-one;
(R)-6-(6-(2-hydroxypropan-2-yl)pyridin-3-yl)-4-(tetrahydrofuran-3-yl)-3,4-dihydropyrazino[2,3-b]pyrazin-2(1H)-one;
(S)-6-(6-(2-hydroxypropan-2-yl)pyridin-3-yl)-4-(tetrahydrofuran-3-yl)-3,4-dihydropyrazino[2,3-b]pyrazin-2(1H)-one;
6-(6-(2-hydroxypropan-2-yl)pyridin-3-yl)-4-phenyl-3,4-dihydropyrazino[2,3-b]pyrazin-2(1H)-one;
(S)-6-(6-(2-hydroxypropan-2-yl)pyridin-3-yl)-3-methyl-4-(2-(tetrahydro-2H-pyran-4-yl)ethyl)-3,4-dihydropyrazino[2,3-b]pyrazin-2(1H)-one;
9-[6-(1-hydroxy-isopropyl)-3-pyridyl]-6,11,4a-trihydromorpholino[4,3-e]pyrazino[2,3-b]pyrazin-5-one;
6-(6-(2-hydroxypropan-2-yl)pyridin-3-yl)-4-((tetrahydro-2H-pyran-4-yl)methyl)-3,4-dihydropyrazino [2,3-b]pyrazin-2(1H)-one;
6-(6-(2-hydroxypropan-2-yl)pyridin-3-yl)-4-(2-methoxyethyl)-3,4-dihydropyrazino[2,3-b]pyrazin-2(1H)-one;
6-(2-amino-7-methyl-1H-benzo[d]imidazol-5-yl)-4-(3-(trifluoromethyl)benzyl)-3,4-dihydropyrazino [2,3-b]pyrazin-2(1H)-one;
6-(6-(2-hydroxypropan-2-yl)pyridin-3-yl)-4-(3-(trifluoromethyl)benzyl)-3,4-dihydropyrazino [2,3-b]pyrazin-2(1H)-one;
9-(4-(4H-1,2,4-triazol-3-yl)-2-methylphenyl)-6,11,4a-trihydromorpholino[4,3-e]pyrazino[2,3-b]pyrazin-5-one;
6-(4-methyl-2-(methylamino)-1H-benzo[d]imidazol-6-yl)-4-(2-(tetrahydro-2H-pyran-4-yl)ethyl)-3,4-dihydropyrazino[2,3-b]pyrazin-2(1H)-one;
8-(4-(4H-1,2,4-triazol-3-yl)-2-methylphenyl)-5,10,3a-trihydropyrazino[2,3-b]pyrrolidino[1,2-e]pyrazin-4-one;
6-(4-(4H-1,2,4-triazol-3-yl)phenyl)-4-ethyl-3,4-dihydropyrazino[2,3-b]pyrazin-2(1H)-one;
6-(4-(4H-1,2,4-triazol-3-yl)phenyl)-4-((tetrahydro-2H-pyran-4-yl)methyl)-3,4-dihydropyrazino [2,3-b]pyrazin-2(1H)-one;
6-(6-(2-hydroxypropan-2-yl)pyridin-3-yl)-4-(2-(tetrahydro-2H-pyran-4-yl)ethyl)-3,4-dihydropyrazino [2,3-b]pyrazin-2(1H)-one;
6-(4-(4H-1,2,4-triazol-3-yl)phenyl)-4-(2-methoxyethyl)-3,4-dihydropyrazino[2,3-b]pyrazin-2(1H)-one;
6-(4-(4H-1,2,4-triazol-3-yl)phenyl)-4-(3-(trifluoromethyl)benzyl)-3,4-dihydropyrazino[2,3-b]pyrazin-2(1H)-one;
6-(2-methyl-4-(4H-1,2,4-triazol-3-yl)phenyl)-4-(2-(tetrahydro-2H-pyran-4-yl)ethyl)-3,4-dihydropyrazino [2,3-b]pyrazin-2(1H)-one;
6-(4-methyl-1H-benzo[d]imidazol-6-yl)-4-(2-(tetrahydro-2H-pyran-4-yl)ethyl)-3,4-dihydropyrazino [2,3-b]pyrazin-2(1H)-one;
6-(4-(2-hydroxypropan-2-yl)phenyl)-4-(2-(tetrahydro-2H-pyran-4-yl)ethyl)-3,4-dihydropyrazino [2,3-b]pyrazin-2(1H)-one; and
6-(4-(1H-1,2,4-triazol-5-yl)phenyl)-4-(2-(tetrahydro-2H-pyran-4-yl)ethyl)-3,4-dihydropyrazino [2,3-b]pyrazin-2(1H)-one,
   and pharmaceutically acceptable salts, clathrates, solvates, stereoisomers, tautomers, and prodrugs thereof.

In one embodiment, the TOR kinase inhibitors include compounds having the following formula (IV):

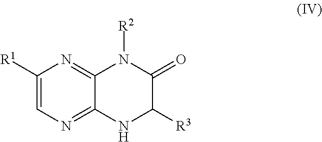

and pharmaceutically acceptable salts, clathrates, solvates, stereoisomers, tautomers, and prodrugs thereof, wherein:

R¹ is substituted or unsubstituted C₁₋₈ alkyl, substituted or unsubstituted aryl, substituted or unsubstituted cycloalkyl, substituted or unsubstituted heterocyclyl, or substituted or unsubstituted heterocyclylalkyl;

R² is H, substituted or unsubstituted C₁₋₈ alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl;

R³ is H, or a substituted or unsubstituted C₁₋₈ alkyl,

wherein in certain embodiments, the TOR kinase inhibitors do not include 7-(4-hydroxyphenyl)-1-(3-methoxybenzyl)-3,4-dihydropyrazino[2,3-b]pyrazin-2(1H)-one, depicted below:

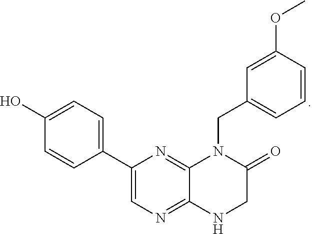 [SMILES: COc1cccc(CN2C(=O)CNc3ncc(-c4ccc(O)cc4)nc32)c1]

In some embodiments of compounds of formula (IV), R¹ is substituted or unsubstituted aryl or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl or pyrazolyl), aminocarbonyl, halogen (for example, fluorine), cyano, hydroxyalkyl and hydroxy. In other embodiments, R¹ is pyridyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl), halogen, aminocarbonyl, cyano, hydroxyalkyl (for example, hydroxypropyl), —OR, and —NR₂, wherein each R is independently H, or a substituted or unsubstituted C₁₋₄ alkyl. In some embodiments, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In some embodiments, R¹ is

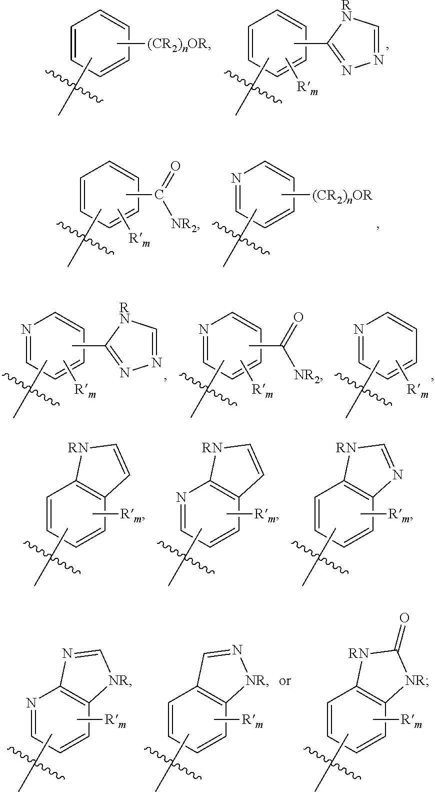

wherein R is at each occurrence independently H, or a substituted or unsubstituted C₁₋₄ alkyl (for example, methyl); R′ is at each occurrence independently a substituted or unsubstituted C₁₋₄ alkyl (for example, methyl), halogen (for example, fluoro), cyano, —OR, or —NR₂; m is 0-3; and n is 0-3. It will be understood by those skilled in the art that any of the substitutuents R′ may be attached to any suitable atom of any of the rings in the fused ring systems.

In some embodiments of compounds of formula (IV), R¹ is

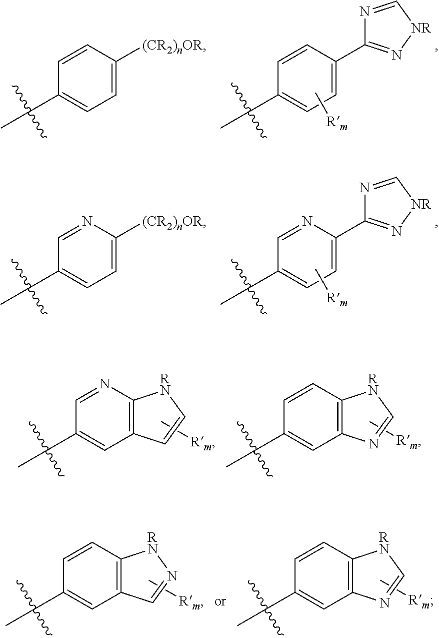

wherein R is at each occurrence independently H, or a substituted or unsubstituted C₁₋₄ alkyl; R′ is at each occurrence independently a substituted or unsubstituted C₁₋₄ alkyl, halogen, cyano, —OR or —NR₂; m is 0-3; and n is 0-3.

In some embodiments of compounds of formula (IV), R² is H, substituted or unsubstituted C₁₋₈ alkyl, substituted or unsubstituted cycloalkyl, substituted or unsubstituted heterocyclyl, substituted or unsubstituted C₁₋₄ alkyl-heterocyclyl, substituted or unsubstituted C₁₋₄ alkyl-aryl, or substituted or unsubstituted C₁₋₄ alkyl-cycloalkyl. For example, R² is H, methyl, ethyl, n-propyl, isopropyl, n-butyl, sec-butyl, isobutyl, tert-butyl, n-pentyl, isopentyl, cyclopentyl, cyclohexyl, tetrahydrofuranyl, tetrahydropyranyl, (C₁₋₄ alkyl)-phenyl, (C₁₋₄ alkyl)-cyclopropyl, (C₁₋₄ alkyl)-cyclobutyl, (C₁₋₄ alkyl)-cyclopentyl, (C₁₋₄ alkyl)-cyclohexyl, (C₁₋₄ alkyl)-pyrrolidyl, (C₁₋₄ alkyl)-piperidyl, (C₁₋₄ alkyl)-piperazinyl, (C₁₋₄ alkyl)-morpholinyl, (C₁₋₄ alkyl)-tetrahydrofuranyl, or (C₁₋₄ alkyl)-tetrahydropyranyl, each optionally substituted.

In other embodiments, R² is H, C₁₋₄ alkyl, (C₁₋₄alkyl)(OR),

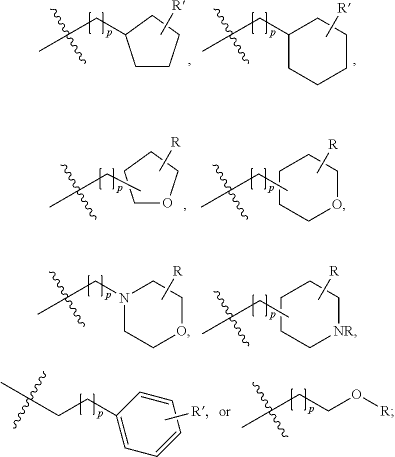

wherein R is at each occurrence independently H, or a substituted or unsubstituted C₁₋₄ alkyl (for example, methyl); R′ is at each occurrence independently H, —OR, cyano, or a substituted or unsubstituted C₁₋₄ alkyl (for example, methyl); and p is 0-3.

In other embodiments of compounds of formula (IV), R² is H, C₁₋₄ alkyl, (C₁₋₄alkyl)(OR),

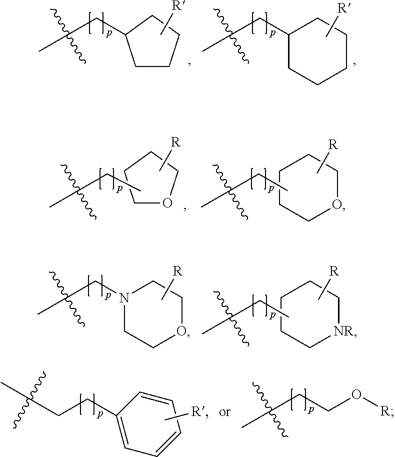

wherein R is at each occurrence independently H, or a substituted or unsubstituted C₁₋₂ alkyl; R′ is at each occurrence independently H, —OR, cyano, or a substituted or unsubstituted C₁₋₂ alkyl; and p is 0-1.

In other embodiments of compounds of formula (IV), R³ is H.

In some such embodiments described herein, R¹ is substituted or unsubstituted aryl, or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridine, pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl, substituted or unsubstituted heterocyclyl, aminocarbonyl, halogen, cyano, hydroxyalkyl and hydroxy. In others, R¹ is pyridyl substituted with one or more substituents independently selected from the group consisting of C₁₋₈ alkyl, substituted or unsubstituted heterocyclyl, halogen, aminocarbonyl, cyano, hydroxyalkyl, —OR, and —NR₂, wherein each R is independently H, or a substituted or unsubstituted C₁₋₄ alkyl. In still others, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In certain embodiments, the compounds of formula (IV) have an R¹ group set forth herein and an R² group set forth herein.

In some embodiments of compounds of formula (IV), the compound at a concentration of 10 μm inhibits mTOR, DNA-PK, PI3K, or a combination thereof by at least about 50%. Compounds of formula (IV) may be shown to be inhibitors of the kinases above in any suitable assay system.

Representative TOR kinase inhibitors of formula (IV) include:
7-(5-fluoro-2-methyl-4-(1H-1,2,4-triazol-3-yl)phenyl)-1-((trans-4-methoxycyclohexyl)methyl)-3,4-dihydropyrazino[2,3-b]pyrazin-2(1H)-one;
7-(6-(1H-1,2,4-triazol-3-yl)pyridin-3-yl)-1-(cis-4-methoxycyclohexyl)-3,4-dihydropyrazino[2,3-b]pyrazin-2(1H)-one;
7-(1H-pyrrolo[2,3-b]pyridin-3-yl)-1-(2-(tetrahydro-2H-pyran-4-yl)ethyl)-3,4-dihydropyrazino[2,3-b]pyrazin-2(1H)-one;
7-(5-fluoro-2-methyl-4-(1H-1,2,4-triazol-3-yl)phenyl)-1-((cis-4-methoxycyclohexyl)methyl)-3,4-dihydropyrazino[2,3-b]pyrazin-2(1H)-one;
1-ethyl-7-(1H-pyrrolo[3,2-b]pyridin-5-yl)-3,4-dihydropyrazino[2,3-b]pyrazin-2(1H)-one;
7-(6-(1H-1,2,4-triazol-3-yl)pyridin-3-yl)-1-((cis-4-methoxycyclohexyl)methyl)-3,4-dihydropyrazino[2,3-b]pyrazin-2(1H)-one;
7-(1H-benzo[d]imidazol-4-yl)-1-(2-(tetrahydro-2H-pyran-4-yl)ethyl)-3,4-dihydropyrazino[2,3-b]pyrazin-2(1H)-one;
7-(1H-pyrrolo[2,3-b]pyridin-4-yl)-1-(2-(tetrahydro-2H-pyran-4-yl)ethyl)-3,4-dihydropyrazino[2,3-b]pyrazin-2(1H)-one;
7-(6-(1H-1,2,4-triazol-3-yl)pyridin-3-yl)-1-((trans-4-methoxycyclohexyl)methyl)-3,4-dihydropyrazino [2,3-b]pyrazin-2(1H)-one;
7-(6-(1H-1,2,4-triazol-3-yl)pyridin-3-yl)-1-((trans-4-hydroxycyclohexyl)methyl)-3,4-dihydropyrazino [2,3-b]pyrazin-2(1H)-one;
7-(6-(1H-1,2,4-triazol-3-yl)pyridin-3-yl)-1-(cis-4-hydroxycyclohexyl)-3,4-dihydropyrazino[2,3-b]pyrazin-2(1H)-one;
7-(5-fluoro-2-methyl-4-(1H-1,2,4-triazol-3-yl)phenyl)-1-(cis-4-hydroxycyclohexyl)-3,4-dihydropyrazino [2,3-b]pyrazin-2(1H)-one;
7-(6-(1H-1,2,4-triazol-3-yl)pyridin-3-yl)-1-(tetrahydro-2H-pyran-4-yl)-3,4-dihydropyrazino[2,3-b]pyrazin-2(1H)-one;
7-(6-(1H-1,2,4-triazol-3-yl)pyridin-3-yl)-1-(2-methoxyethyl)-3,4-dihydropyrazino[2,3-b]pyrazin-2(1H)-one;
7-(6-(1H-1,2,4-triazol-3-yl)pyridin-3-yl)-1-ethyl-3,4-dihydropyrazino[2,3-b]pyrazin-2(1H)-one;
7-(5-fluoro-2-methyl-4-(1H-1,2,4-triazol-3-yl)phenyl)-1-((cis-4-hydroxycyclohexyl)methyl)-3,4-dihydropyrazino [2,3-b]pyrazin-2(1H)-one;
7-(5-fluoro-2-methyl-4-(1H-1,2,4-triazol-3-yl)phenyl)-1-(tetrahydro-2H-pyran-4-yl)-3,4-dihydropyrazino [2,3-b]pyrazin-2(1H)-one;
7-(1H-indol-4-yl)-1-(2-(tetrahydro-2H-pyran-4-yl)ethyl)-3,4-dihydropyrazino[2,3-b]pyrazin-2(1H)-one;
7-(5-fluoro-2-methyl-4-(1H-1,2,4-triazol-3-yl)phenyl)-1-((trans-4-hydroxycyclohexyl)methyl)-3,4-dihydropyrazino[2,3-b]pyrazin-2(1H)-one;
7-(6-(1H-1,2,4-triazol-3-yl)pyridin-3-yl)-1-((cis-4-hydroxycyclohexyl)methyl)-3,4-dihydropyrazino [2,3-b]pyrazin-2(1H)-one;
7-(6-(1H-1,2,4-triazol-3-yl)pyridin-3-yl)-1-(trans-4-hydroxycyclohexyl)-3,4-dihydropyrazino [2,3-b]pyrazin-2(1H)-one;
7-(6-(1H-1,2,4-triazol-3-yl)pyridin-3-yl)-1-(trans-4-methoxycyclohexyl)-3,4-dihydropyrazino [2,3-b]pyrazin-2(1H)-one;
7-(6-(1H-1,2,4-triazol-3-yl)pyridin-3-yl)-1-isopropyl-3,4-dihydropyrazino[2,3-b]pyrazin-2(1H)-one;
7-(5-fluoro-2-methyl-4-(1H-1,2,4-triazol-3-yl)phenyl)-1-(trans-4-methoxycyclohexyl)-3,4-dihydropyrazino [2,3-b]pyrazin-2(1H)-one;
7-(5-fluoro-2-methyl-4-(1H-1,2,4-triazol-3-yl)phenyl)-1-(trans-4-hydroxycyclohexyl)-3,4-dihydropyrazino [2,3-b]pyrazin-2(1H)-one;
7-(5-fluoro-2-methyl-4-(1H-1,2,4-triazol-3-yl)phenyl)-1-(2-methoxyethyl)-3,4-dihydropyrazino [2,3-b]pyrazin-2(1H)-one;
7-(5-fluoro-2-methyl-4-(1H-1,2,4-triazol-3-yl)phenyl)-1-isopropyl-3,4-dihydropyrazino[2,3-b]pyrazin-2(1H)-one;
1-ethyl-7-(5-fluoro-2-methyl-4-(1H-1,2,4-triazol-3-yl)phenyl)-3,4-dihydropyrazino[2,3-b]pyrazin-2(1H)-one;
7-(2-hydroxypyridin-4-yl)-1-(2-(tetrahydro-2H-pyran-4-yl)ethyl)-3,4-dihydropyrazino[2,3-b]pyrazin-2(1H)-one;
1-isopropyl-7-(4-methyl-6-(1H-1,2,4-triazol-3-yl)pyridin-3-yl)-3,4-dihydropyrazino[2,3-b]pyrazin-2(1H)-one;
5-(8-isopropyl-7-oxo-5,6,7,8-tetrahydropyrazino[2,3-b]pyrazin-2-yl)-4-methylpicolinamide;
7-(1H-indazol-4-yl)-1-(2-(tetrahydro-2H-pyran-4-yl)ethyl)-3,4-dihydropyrazino[2,3-b]pyrazin-2(1H)-one;
7-(2-aminopyrimidin-5-yl)-1-(2-(tetrahydro-2H-pyran-4-yl)ethyl)-3,4-dihydropyrazino[2,3-b]pyrazin-2(1H)-one;
7-(2-aminopyridin-4-yl)-1-(2-(tetrahydro-2H-pyran-4-yl)ethyl)-3,4-dihydropyrazino[2,3-b]pyrazin-2(1H)-one;
7-(6-(methylamino)pyridin-3-yl)-1-(2-(tetrahydro-2H-pyran-4-yl)ethyl)-3,4-dihydropyrazino[2,3-b]pyrazin-2(1H)-one;
7-(6-hydroxypyridin-3-yl)-1-(2-(tetrahydro-2H-pyran-4-yl)ethyl)-3,4-dihydropyrazino[2,3-b]pyrazin-2(1H)-one;
7-(4-(1H-pyrazol-3-yl)phenyl)-1-(2-methoxyethyl)-3,4-dihydropyrazino[2,3-b]pyrazin-2(1H)-one;
7-(pyridin-3-yl)-1-(2-(tetrahydro-2H-pyran-4-yl)ethyl)-3,4-dihydropyrazino[2,3-b]pyrazin-2(1H)-one;
7-(1H-indazol-4-yl)-1-(2-methoxyethyl)-3,4-dihydropyrazino[2,3-b]pyrazin-2(1H)-one;
7-(1H-indazol-6-yl)-1-(2-methoxyethyl)-3,4-dihydropyrazino[2,3-b]pyrazin-2(1H)-one;
7-(pyrimidin-5-yl)-1-(2-(tetrahydro-2H-pyran-4-yl)ethyl)-3,4-dihydropyrazino[2,3-b]pyrazin-2(1H)-one;
7-(6-methoxypyridin-3-yl)-1-(2-(tetrahydro-2H-pyran-4-yl)ethyl)-3,4-dihydropyrazino[2,3-b]pyrazin-2(1H)-one;
1-(2-methoxyethyl)-7-(1H-pyrrolo[2,3-b]pyridin-5-yl)-3,4-dihydropyrazino[2,3-b]pyrazin-2(1H)-one;
1-ethyl-7-(1H-pyrrolo[2,3-b]pyridin-5-yl)-3,4-dihydropyrazino[2,3-b]pyrazin-2(1H)-one;
1-ethyl-7-(1H-indazol-4-yl)-3,4-dihydropyrazino[2,3-b]pyrazin-2(1H)-one;
7-(pyridin-4-yl)-1-(2-(tetrahydro-2H-pyran-4-yl)ethyl)-3,4-dihydropyrazino[2,3-b]pyrazin-2(1H)-one;
7-(6-aminopyridin-3-yl)-1-(2-(tetrahydro-2H-pyran-4-yl)ethyl)-3,4-dihydropyrazino[2,3-b]pyrazin-2(1H)-one;
1-methyl-7-(2-methyl-6-(4H-1,2,4-triazol-3-yl)pyridin-3-yl)-3,4-dihydropyrazino[2,3-b]pyrazin-2(1H)-one;
2-(2-hydroxypropan-2-yl)-5-(8-(trans-4-methoxycyclohexyl)-7-oxo-5,6,7,8-tetrahydropyrazino[2,3-b]pyrazin-2-yl)pyridine 1-oxide;
4-methyl-5-(7-oxo-8-((tetrahydro-2H-pyran-4-yl)methyl)-5,6,7,8-tetrahydropyrazino[2,3-b]pyrazin-2-yl)picolinamide;
5-(8-((cis-4-methoxycyclohexyl)methyl)-7-oxo-5,6,7,8-tetrahydropyrazino[2,3-b]pyrazin-2-yl)-4-methylpicolinamide;
7-(1H-pyrazol-4-yl)-1-(2-(tetrahydro-2H-pyran-4-yl)ethyl)-3,4-dihydropyrazino[2,3-b]pyrazin-2(1H)-one;
1-(trans-4-methoxycyclohexyl)-7-(4-methyl-6-(1H-1,2,4-triazol-3-yl)pyridin-3-yl)-3,4-dihydropyrazino[2,3-b]pyrazin-2(1H)-one;
3-((7-(2-methyl-6-(4H-1,2,4-triazol-3-yl)pyridin-3-yl)-2-oxo-3,4-dihydropyrazino[2,3-b]pyrazin-1(2H)-yl)methyl)benzonitrile;
1-((trans-4-methoxycyclohexyl)methyl)-7-(4-methyl-6-(1H-1,2,4-triazol-3-yl)pyridin-3-yl)-3,4-dihydropyrazino[2,3-b]pyrazin-2(1H)-one;
3-(7-oxo-8-(2-(tetrahydro-2H-pyran-4-yl)ethyl)-5,6,7,8-tetrahydropyrazino[2,3-b]pyrazin-2-yl)benzamide;
5-(8-((trans-4-methoxycyclohexyl)methyl)-7-oxo-5,6,7,8-tetrahydropyrazino[2,3-b]pyrazin-2-yl)-4-methylpicolinamide;
3-((7-(6-(2-hydroxypropan-2-yl)pyridin-3-O-2-oxo-3,4-dihydropyrazino[2,3-b]pyrazin-(2H)-yl)methyl)benzonitrile;
7-(6-(2-hydroxypropan-2-yl)pyridin-3-yl)-1-((1R,3R)-3-methoxycyclopentyl)-3,4-dihydropyrazino[2,3-b]pyrazin-2(1H)-one;
7-(6-(2-hydroxypropan-2-yl)pyridin-3-yl)-1-((1S,3R)-3-methoxycyclopentyl)-3,4-dihydropyrazino[2,3-b]pyrazin-2(1H)-one;
7-(6-(2-hydroxypropan-2-yl)pyridin-3-yl)-1-((1S,3S)-3-methoxycyclopentyl)-3,4-dihydropyrazino[2,3-b]pyrazin-2(1H)-one;
7-(6-(2-hydroxypropan-2-yl)pyridin-3-yl)-1-((1R,3S)-3-methoxycyclopentyl)-3,4-dihydropyrazino[2,3-b]pyrazin-2(1H)-one;
7-(1H-indazol-6-yl)-1-(2-(tetrahydro-2H-pyran-4-yl)ethyl)-3,4-dihydropyrazino[2,3-b]pyrazin-2(1H)-one;
7-(2-methyl-6-(4H-1,2,4-triazol-3-yl)pyridin-3-yl)-1-(2-morpholinoethyl)-3,4-dihydropyrazino[2,3-b]pyrazin-2(1H)-one;
1-(trans-4-hydroxycyclohexyl)-7-(2-methyl-6-(4H-1,2,4-triazol-3-yl)pyridin-3-yl)-3,4-dihydropyrazino[2,3-b]pyrazin-2(1H)-one;
1-(cis-4-hydroxycyclohexyl)-7-(2-methyl-6-(4H-1,2,4-triazol-3-yl)pyridin-3-yl)-3,4-dihydropyrazino[2,3-b]pyrazin-2(1H)-one;
7-(6-(2-hydroxypropan-2-yl)pyridin-3-yl)-1-(2-morpholinoethyl)-3,4-dihydropyrazino[2,3-b]pyrazin-2(1H)-one;
1-isopropyl-7-(2-methyl-6-(4H-1,2,4-triazol-3-yl)pyridin-3-yl)-3,4-dihydropyrazino[2,3-b]pyrazin-2(1H)-one;
7-(1H-imidazo[4,5-b]pyridin-6-yl)-1-(2-(tetrahydro-2H-pyran-4-yl)ethyl)-3,4-dihydropyrazino[2,3-b]pyrazin-2(1H)-one;
1-((cis-4-methoxycyclohexyl)methyl)-7-(2-methyl-6-(1H-1,2,4-triazol-3-yl)pyridin-3-yl)-3,4-dihydropyrazino[2,3-b]pyrazin-2(1H)-one;
1-(trans-4-hydroxycyclohexyl)-7-(6-(2-hydroxypropan-2-yl)pyridin-3-yl)-3,4-dihydropyrazino[2,3-b]pyrazin-2(1H)-one;
1-(cis-4-hydroxycyclohexyl)-7-(6-(2-hydroxypropan-2-yl)pyridin-3-yl)-3,4-dihydropyrazino[2,3-b]pyrazin-2(1H)-one;
4-(7-oxo-8-(2-(tetrahydro-2H-pyran-4-yl)ethyl)-5,6,7,8-tetrahydropyrazino[2,3-b]pyrazin-2-yl)benzamide;
7-(1H-indazol-5-yl)-1-(2-(tetrahydro-2H-pyran-4-yl)ethyl)-3,4-dihydropyrazino[2,3-b]pyrazin-2(1H)-one;
7-(1H-pyrrolo[2,3-b]pyridin-5-yl)-1-(2-(tetrahydro-2H-pyran-4-yl)ethyl)-3,4-dihydropyrazino[2,3-b]pyrazin-2(1H)-one;
7-(2-methyl-6-(4H-1,2,4-triazol-3-yl)pyridin-3-yl)-1-(tetrahydro-2H-pyran-4-yl)-3,4-dihydropyrazino [2,3-b]pyrazin-2(1H)-one;
1-((1S,3R)-3-methoxycyclopentyl)-7-(2-methyl-6-(4H-1,2,4-triazol-3-yl)pyridin-3-yl)-3,4-dihydropyrazino [2,3-b]pyrazin-2(1H)-one;
1-((1R,3R)-3-methoxycyclopentyl)-7-(2-methyl-6-(4H-1,2,4-triazol-3-yl)pyridin-3-yl)-3,4-dihydropyrazino [2,3-b]pyrazin-2(1H)-one;
1-((1R,3 S)-3-methoxycyclopentyl)-7-(2-methyl-6-(4H-1,2,4-triazol-3-yl)pyridin-3-yl)-3,4-dihydropyrazino [2,3-b]pyrazin-2(1H)-one;
1-((1S,3 S)-3-methoxycyclopentyl)-7-(2-methyl-6-(4H-1,2,4-triazol-3-yl)pyridin-3-yl)-3,4-dihydropyrazino [2,3-b]pyrazin-2(1H)-one;
7-(1H-indol-5-yl)-1-(2-(tetrahydro-2H-pyran-4-yl)ethyl)-3,4-dihydropyrazino[2,3-b]pyrazin-2(1H)-one;
1-ethyl-7-(2-methyl-6-(4H-1,2,4-triazol-3-yl)pyridin-3-yl)-3,4-dihydropyrazino[2,3-b]pyrazin-2(1H)-one;
7-(1H-indol-6-yl)-1-(2-(tetrahydro-2H-pyran-4-yl)ethyl)-3,4-dihydropyrazino[2,3-b]pyrazin-2(1H)-one;
7-(4-(2-hydroxypropan-2-yl)phenyl)-1-(trans-4-methoxycyclohexyl)-3,4-dihydropyrazino[2,3-b]pyrazin-2(1H)-one;
7-(6-(2-hydroxypropan-2-yl)pyridin-3-yl)-1-(tetrahydro-2H-pyran-4-yl)-3,4-dihydropyrazino [2,3-b]pyrazin-2(1H)-one;
1-((trans-4-methoxycyclohexyl)methyl)-7-(2-methyl-6-(1H-1,2,4-triazol-3-yl)pyridin-3-yl)-3,4-dihydropyrazino [2,3-b]pyrazin-2(1H)-one;
7-(6-(2-hydroxypropan-2-yl)pyridin-3-yl)-1-((cis-4-methoxycyclohexyl)methyl)-3,4-dihydropyrazino [2,3-b]pyrazin-2(1H)-one;
1-(2-methoxyethyl)-7-(4-methyl-2-(methylamino)-1H-benzo[d]imidazol-6-yl)-3,4-dihydropyrazino [2,3-b]pyrazin-2(1H)-one;
7-(7-methyl-2-oxo-2,3-dihydro-1H-benzo[d]imidazol-5-yl)-1-((tetrahydro-2H-pyran-4-yl)methyl)-3,4-dihydropyrazino[2,3-b]pyrazin-2(1H)-one;
7-(2-methyl-4-(4H-1,2,4-triazol-3-yl)phenyl)-3,4-dihydropyrazino[2,3-b]pyrazin-2(1H)-one;
1-(2-methoxyethyl)-7-(4-methyl-6-(1H-1,2,4-triazol-3-yl)pyridin-3-yl)-3,4-dihydropyrazino[2,3-b]pyrazin-2(1H)-one;
1-benzyl-7-(2-methyl-4-(4H-1,2,4-triazol-3-yl)phenyl)-3,4-dihydropyrazino[2,3-b]pyrazin-2(1H)-one;
7-(3-fluoro-4-(4H-1,2,4-triazol-3-yl)phenyl)-1-(2-methoxyethyl)-3,4-dihydropyrazino[2,3-b]pyrazin-2(1H)-one;
7-(3-fluoro-4-(4H-1,2,4-triazol-3-yl)phenyl)-1-(2-(tetrahydro-2H-pyran-4-yl)ethyl)-3,4-dihydropyrazino[2,3-b]pyrazin-2(1H)-one;
7-(3-fluoro-2-methyl-4-(1H-1,2,4-triazol-3-yl)phenyl)-1-(2-methoxyethyl)-3,4-dihydropyrazino[2,3-b]pyrazin-2(1H)-one;
1-(trans-4-methoxycyclohexyl)-7-(2-methyl-6-(4H-1,2,4-triazol-3-yl)pyridin-3-yl)-3,4-dihydropyrazino[2,3-b]pyrazin-2(1H)-one;
7-(6-(2-hydroxypropan-2-yl)pyridin-3-yl)-1-(trans-4-methoxycyclohexyl)-3,4-dihydropyrazino[2,3-b]pyrazin-2(1H)-one;
7-(5-fluoro-2-methyl-4-(4H-1,2,4-triazol-3-yl)phenyl)-1-(2-(tetrahydro-2H-pyran-4-yl)ethyl)-3,4-dihydropyrazino[2,3-b]pyrazin-2(1H)-one;
7-(3-fluoro-2-methyl-4-(1H-1,2,4-triazol-3-yl)phenyl)-1-(2-(tetrahydro-2H-pyran-4-yl)ethyl)-3,4-dihydropyrazino[2,3-b]pyrazin-2(1H)-one;
1-(2-methoxyethyl)-7-(2-methyl-6-(4H-1,2,4-triazol-3-yl)pyridin-3-yl)-3,4-dihydropyrazino[2,3-b]pyrazin-2(1H)-one;
7-(6-(2-hydroxypropan-2-yl)pyridin-3-yl)-1-((trans-4-methoxycyclohexyl)methyl)-3,4-dihydropyrazino[2,3-b]pyrazin-2(1H)-one;
1-(cyclopentylmethyl)-7-(6-(2-hydroxypropan-2-yl)pyridin-3-yl)-3,4-dihydropyrazino[2,3-b]pyrazin-2(1H)-one;
7-(4-(2-hydroxypropan-2-yl)phenyl)-1-(2-methoxyethyl)-3,4-dihydropyrazino[2,3-b]pyrazin-2(1H)-one;
(S)-7-(6-(1-hydroxyethyl)pyridin-3-yl)-1-(2-(tetrahydro-2H-pyran-4-yl)ethyl)-3,4-dihydropyrazino [2,3-b]pyrazin-2(1H)-one;
(R)-7-(6-(1-hydroxyethyl)pyridin-3-yl)-1-(2-(tetrahydro-2H-pyran-4-yl)ethyl)-3,4-dihydropyrazino [2,3-b]pyrazin-2(1H)-one;
7-(2-methyl-6-(4H-1,2,4-triazol-3-yl)pyridin-3-yl)-1-((tetrahydro-2H-pyran-4-yl)methyl)-3,4-dihydropyrazino [2,3-b]pyrazin-2(1H)-one;
7-(4-(2-hydroxypropan-2-yl)phenyl)-1-(2-(tetrahydro-2H-pyran-4-yl)ethyl)-3,4-dihydropyrazino [2,3-b]pyrazin-2(1H)-one;
7-(6-(2-hydroxypropan-2-yl)pyridin-3-yl)-1-(4-(trifluoromethyl)benzyl)-3,4-dihydropyrazino [2,3-b]pyrazin-2(1H)-one;
7-(6-(2-hydroxypropan-2-yl)pyridin-3-yl)-1-(3-(trifluoromethyl)benzyl)-3,4-dihydropyrazino [2,3-b]pyrazin-2(1H)-one;
7-(6-(2-hydroxypropan-2-yl)pyridin-3-yl)-1-(3-methoxypropyl)-3,4-dihydropyrazino[2,3-b]pyrazin-2(1H)-one;
7-(4-methyl-6-(1H-1,2,4-triazol-3-yl)pyridin-3-yl)-1-(2-(tetrahydro-2H-pyran-4-yl)ethyl)-3,4-dihydropyrazino [2,3-b]pyrazin-2(1H)-one;
7-(6-(2-hydroxypropan-2-yl)pyridin-3-yl)-1-(2-methoxyethyl)-3,4-dihydropyrazino[2,3-b]pyrazin-2(1H)-one;
7-(6-(2-hydroxypropan-2-yl)pyridin-3-yl)-1-((tetrahydro-2H-pyran-4-yl)methyl)-3,4-dihydropyrazino [2,3-b]pyrazin-2(1H)-one;
7-(4-methyl-2-(methylamino)-1H-benzo[d]imidazol-6-yl)-1-((tetrahydro-2H-pyran-4-yl)methyl)-3,4-dihydropyrazino[2,3-b]pyrazin-2(1H)-one;
7-(2-amino-4-methyl-1H-benzo[d]imidazol-6-yl)-1-((tetrahydro-2H-pyran-4-yl)methyl)-3,4-dihydropyrazino [2,3-b]pyrazin-2(1H)-one;
7-(2-methyl-6-(4H-1,2,4-triazol-3-yl)pyridin-3-yl)-1-(2-(tetrahydro-2H-pyran-4-yl)ethyl)-3,4-dihydropyrazino [2,3-b]pyrazin-2(1H)-one;
(R)-7-(6-(2-hydroxypropan-2-yl)pyridin-3-yl)-3-methyl-1-(2-(tetrahydro-2H-pyran-4-yl)ethyl)-3,4-dihydropyrazino[2,3-b]pyrazin-2(1H)-one;
(S)-7-(6-(2-hydroxypropan-2-yl)pyridin-3-yl)-3-methyl-1-(2-(tetrahydro-2H-pyran-4-yl)ethyl)-3,4-dihydropyrazino[2,3-b]pyrazin-2(1H)-one;
7-(6-(2-hydroxypropan-2-yl)pyridin-3-yl)-3,3-dimethyl-1-(2-(tetrahydro-2H-pyran-4-yl)ethyl)-3,4-dihydropyrazino[2,3-b]pyrazin-2(1H)-one;
7-(2-amino-4-methyl-1H-benzo[d]imidazol-6-yl)-1-(2-(tetrahydro-2H-pyran-4-yl)ethyl)-3,4-dihydropyrazino [2,3-b]pyrazin-2(1H)-one;
7-(6-(2-hydroxypropan-2-yl)pyridin-3-yl)-1-(2-(tetrahydro-2H-pyran-4-yl)ethyl)-3,4-dihydropyrazino [2,3-b]pyrazin-2(1H)-one;
7-(2-methyl-4-(1H-1,2,4-triazol-3-yl)phenyl)-1-(2-(tetrahydro-2H-pyran-4-yl)ethyl)-3,4-dihydropyrazino [2,3-b]pyrazin-2(1H)-one;
7-(4-(1H-1,2,4-triazol-5-yl)phenyl)-1-(2-(tetrahydro-2H-pyran-4-yl)ethyl)-3,4-dihydropyrazino [2,3-b]pyrazin-2(1H)-one;
1-(1-hydroxypropan-2-yl)-7-(2-methyl-6-(1H-1,2,4-triazol-3-yl)pyridin-3-yl)-3,4-dihydropyrazino [2,3-b]pyrazin-2(1H)-one; and
1-(2-hydroxyethyl)-7-(2-methyl-6-(1H-1,2,4-triazol-3-yl)pyridin-3-yl)-3,4-dihydropyrazino[2,3-b]pyrazin-2(1H)-one,
   and pharmaceutically acceptable salts, clathrates, solvates, stereoisomers, tautomers, and prodrugs thereof.

In one embodiment, the TOR kinase inhibitor is a compound having the following formula:

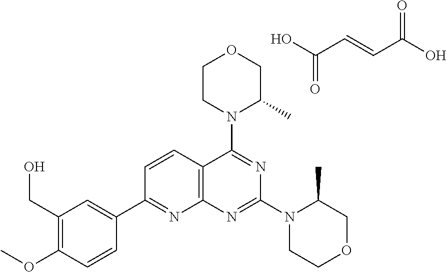

or a pharmaceutically acceptable salt, clathrate, solvate, stereoisomer, tautomer, or prodrug thereof.

In one embodiment, the TOR kinase inhibitor is a compound having the following formula:

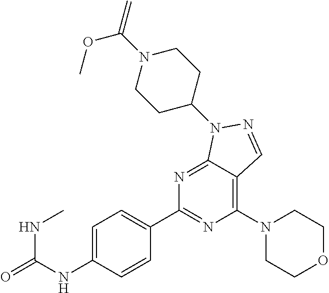 [SMILES: C=C(OC)N1CCC(n2ncc3c(N4CCOCC4)nc(-c4ccc(NC(=O)NC)cc4)nc32)CC1]

or a pharmaceutically acceptable salt, clathrate, solvate, stereoisomer, tautomer, or prodrug thereof.

In one embodiment, the TOR kinase inhibitor is a compound having the following formula:

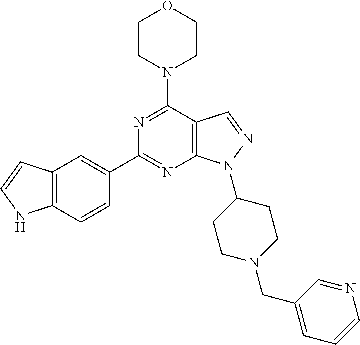 [SMILES: c1cncc(CN2CCC(n3ncc4c(N5CCOCC5)nc(-c5ccc6[nH]ccc6c5)nc43)CC2)c1]

or a pharmaceutically acceptable salt, clathrate, solvate, stereoisomer, tautomer, or prodrug thereof.

In one embodiment, the TOR kinase inhibitor is a compound having the following formula:

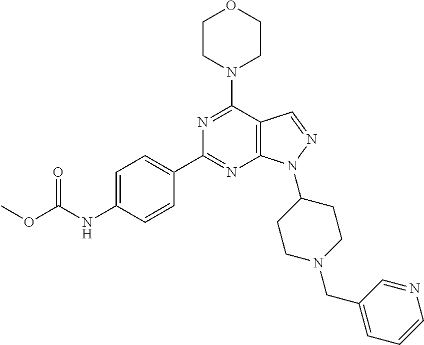 [SMILES: COC(=O)Nc1ccc(-c2nc(N3CCOCC3)c3cnn(C4CCN(Cc5cccnc5)CC4)c3n2)cc1]

or a pharmaceutically acceptable salt, clathrate, solvate, stereoisomer, tautomer, or prodrug thereof.

In one embodiment, the TOR kinase inhibitor is a compound having the following formula:

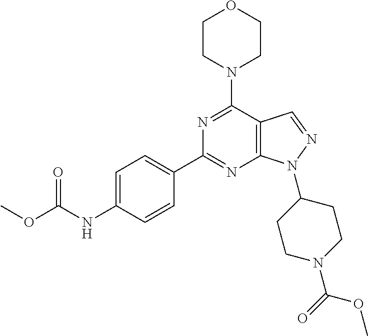

or a pharmaceutically acceptable salt, clathrate, solvate, stereoisomer, tautomer, or prodrug thereof.

In one embodiment, the TOR kinase inhibitor is a compound having the following formula:

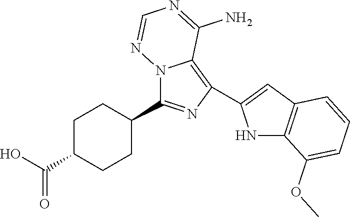 [SMILES: COc1cccc2cc(-c3nc([C@H]4CC[C@H](C(=O)O)CC4)n4ncnc(N)c34)[nH]c12]

or a pharmaceutically acceptable salt, clathrate, solvate, stereoisomer, tautomer, or prodrug thereof.

In one embodiment, the TOR kinase inhibitor is a compound having the following formula:

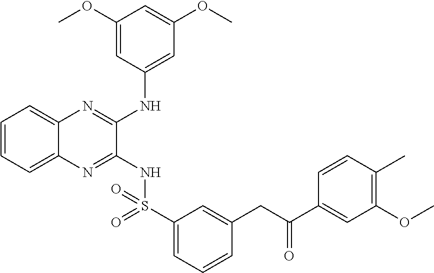 [SMILES: COc1cc(Nc2nc3ccccc3nc2NS(=O)(=O)c2cccc(CC(=O)c3ccc(C)c(OC)c3)c2)cc(OC)c1]

or a pharmaceutically acceptable salt, clathrate, solvate, stereoisomer, tautomer, or prodrug thereof.

In one embodiment, the TOR kinase inhibitor is a compound having the following formula:

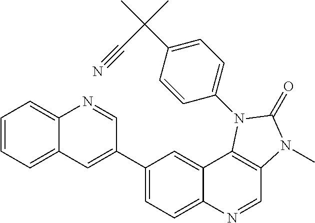 [SMILES: Cn1c(=O)n(-c2ccc(C(C)(C)C#N)cc2)c2c3cc(-c4cnc5ccccc5c4)ccc3ncc21]

or a pharmaceutically acceptable salt, clathrate, solvate, stereoisomer, tautomer, or prodrug thereof.

In one embodiment, the TOR kinase inhibitor is a compound disclosed in WO 2008/023161 (see, e.g., page 5, line 5 to page 11, line 15), WO 2009/007751 (see, e.g., page 9, line 8 to page 26, line 8), WO 2009/007749 (see, e.g., page 9, line 21 to page 29, line 23), WO 2009/007750 (see, e.g., page 9, line 21 to page 32, line 22), WO 2009/007748 (see, e.g., page 9, line 6 to page 42, line 28), WO 2008/032028 (see, e.g., page 11, line 13 to page 21, line 13), WO 2008/032086 (see, e.g., page 10 line 21 to page 15, line 22), WO 2008/032072 (see, e.g., page 11, line 11 to page 16, line 13), WO 2008/032033 (see, e.g., page 11, line 3 to page 16, line 5), WO 2008/032089 (see, e.g., page 11, line 11 to page 16, line 13), WO 2008/032060 (see, e.g., page 11, line 3 to page page 16, line 6), WO 2008/032091 (see, e.g., page 11, line 11 to page 16, line 13), WO 2008/032036 (see, e.g., page 11, line 13 to page 21, line 13), WO 2008/032077 (see, e.g., page 10, line 21 to page 15, line 22), WO 2008/032064 (see, e.g., page 11, line 3 to page 16, line 5), WO 2008/032027 (see, e.g., page 10, line 21 to page 15, line 22), WO 2007/135398 (see, e.g., page 11, line 28 to page 16, line 6), WO 2007/129052 (see, e.g., page 10, line 8 to page 13, line 5), WO 2007/129044 (see, e.g., page 10, line 22 to page 13, line 20), WO 2007/080382 (see, e.g., page 9, line 20 to page 32, line 32), WO 2007/066102 (see, e.g., page 9, line 22 to page 14, line 17), WO 2007/066099 (see, e.g., page 9, line 22 to page 14, line 14), WO 2007/066103 (see, e.g., page 9, line 22 to page 14, line 16), WO 2007/060404 (see, e.g., 5, line 4 to page 7, line 25), WO 2006/090169 (see, e.g., page 4, lines 1-25), WO 2006/090167 (see, e.g., page 3, line 33 to page 6, line 23), WO 2008/115974 (see, e.g., page 4, paragraph [0012] to page 127, paragraph [0257]), WO 2009/052145 (see, e.g., page 5, paragraph [0015] to page 81, paragraph [0082]), WO 2010/006072 (see, e.g., page 28, line 1 to page 34, line 1), WO 2007/044698 (see, e.g., page 3, paragraph [0010] to the bottom of page 7), WO 2007/044813 (see, e.g., page 3, paragraph [0010] to the middle of page 7), WO 2007/044729 (see, e.g., page 3, paragraph [0010] to the bottom of page 10), WO 2007/129161 (see, e.g., page 2, line 10 to page 9, line 19), WO 2006/046031 (see, e.g., page 2, line 15 to page 4, line 12), WO 2003/072557 (see, e.g., page 1, line 4 to page 2, line 27), WO 2004/048365 (see, e.g., page 1, line 4 to page 4, line 17), WO 2004/078754 (see, e.g., page 1, line 4 to page 2, line 21), WO 2004/096797 (see, e.g., page 1, line 4 to page 2, line 34), WO 2005/021519 (see, e.g., page 1, line 4 to page 4, line 17) or US 2007/112005 (see, e.g., page 2, paragraph [0012] to page 22, paragraph [0065]), each of which is incorporated by reference herein in its entirety.

### 5.4 Methods for Making TOR Kinase Inhibitors

The TOR kinase inhibitors can be obtained via standard, well-known synthetic methodology, see e.g., March, J. Advanced Organic Chemistry; Reactions Mechanisms, and Structure, 4th ed., 1992. Starting materials useful for preparing compounds of formula (III) and intermediates therefore, are commercially available or can be prepared from commercially available materials using known synthetic methods and reagents.

Particular methods for preparing compounds of formula (I) are disclosed in U.S. application Ser. No. 11/975,652, filed Oct. 18, 2007, incorporated by reference herein in its entirety. Particular methods for preparing compounds of formula (II) are disclosed in U.S. application Ser. No. 11/975,657, filed Oct. 18, 2007, incorporated by reference herein in its entirety. Particular methods for preparing compounds of formula (III) and (IV) are disclosed in U.S. application Ser. No. 12/605,791, filed Oct. 26, 2009, incorporated by reference herein in its entirety.

### 5.5 Methods of Use

Without being limited by theory, it is believed that LKB1 plays an important role in the nutrient sensing arm of the mTOR pathway. In particular, it is believed that LKB1 is a negative regulator of the mTOR pathway under stress conditions, such as hypoxia and low glucose. LKB1 suppresses mTOR activity via its downsteam kinase, AMP-activated protein kinase (AMPK). In response to energy stress, LKB1 phosphorylates the AMPK catalytic subunit at T172 and this phosphorylation is essential for activation of AMPK. Activated AMPK phosphorylates TSC2 and raptor, and suppresses mTOR activity (Shackelford D B and Shaw J S, Nat. Rev Cancer 9:563 (2009)). Therefore, phosphorylation or activity of AMPK can be used as a marker for LKB1 status. In basal conditions, it is believed that loss of LKB1 and/or AMPK can result in activation of the mTOR pathway. In cancer cells, under stress conditions, it is believed that the LKB1/AMPK pathway may actually play a protective role by causing cells to slow down their proliferation and thus evade apoptosis induced by the stress condition. However, it is believed that in LKB1 mutant cancer cells (e.g., cells harboring a LKB1 gene mutation resulting in a decrease in LKB1 mRNA expression, a decrease in LKB1 protein production or a non-functional LKB1 protein), in the absence of the negative signal to mTOR, the cancer cells continue to proliferate and undergo metabolic catastrophe. Accordingly, without being limited by theory, it is believed that TOR kinase inhibitors by their effects on cell metabolism cause a stress response in cancer cells and in LKB1 mutant cancer cells, and in the absence of a negative signal to slow the growth of the cells, result in cell death. Also without being limited by theory, it is believed that the expression levels of certain genes are characteristic of LKB1 gene or protein mutation or loss, such that measurement of the gene expression levels of a biological sample can be used to predict LKB1 status of the biological sample.

Provided herein are methods for predicting the LKB1 status of a patient or a biological sample, comprising the measurement of a predictive gene expression level. Without being limited by theory, it is believed that certain gene expression levels are characteristic of LKB1 gene and/or protein mutation and/or loss.

Further provided herein are methods for treating or preventing a cancer, for example non-small cell lung carcinoma or cervical cancer, or treating a tumor syndrome, for example Peutz-Jeghers Syndrome, comprising administering an effective amount of a TOR kinase inhibitor to a patient having a cancer or a tumor syndrome characterized by a particular gene expression level, relative to that of wild type.

Further provided herein are methods for treating or preventing a cancer, for example non-small cell lung carcinoma or cervical cancer, comprising screening a patient's cancer for the presence of a particular gene expression level relative to that of wild type and administering an effective amount of a TOR kinase inhibitor to the patient having a cancer characterized by a particular gene expression level.

Further provided herein are methods for predicting LKB1 gene and/or protein loss and/or mutation in a patient's (“test patient”) cancer, for example non-small cell lung carcinoma or cervical cancer, comprising: a) obtaining a biological test sample from the patient's cancer; b) obtaining the gene expression level(s) of one or more genes selected from Table 1 in said biological sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of an LKB1 gene and/or protein loss and/or mutation in the patient's cancer.

Further provided herein are methods for treating non-small cell lung carcinoma, cervical cancer or Peutz-Jeghers Syndrome, comprising administering an effective amount of a TOR kinase inhibitor to a patient having non-small cell lung carcinoma, cervical cancer or Peutz-Jeghers Syndrome, wherein the gene expression level(s) of a biological test sample from said patient is characterized by higher similarity to the gene expression level of a reference sample with LKB1 gene and/or protein loss and/or mutation than the gene expression level of a wild type sample without LKB1 gene and/or protein loss and/or mutation, and wherein the genes are selected from Table 1.

Further provided are methods for treating non-small cell lung carcinoma or cervical cancer, comprising screening a patient's carcinoma or cancer for the presence of LKB1 gene and/or protein loss and/or mutation, relative to wild type, and administering an effective amount of a TOR kinase inhibitor to the patient having non-small cell lung carcinoma or cervical cancer characterized by a gene expression level(s) characterized by higher similarity to the gene expression level of a reference sample with LKB1 gene and/or protein loss and/or mutation than the gene expression level of a wild type sample without LKB1 gene and/or protein loss and/or mutation, and wherein the genes are selected from Table 1.

Further provided herein are methods for predicting response to treatment with a TOR kinase inhibitor in a patient, the method comprising: a) obtaining a biological test sample from the patient's cancer; b) obtaining the gene expression level(s) of one or more genes selected from Table 1 in said biological test sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of response to TOR kinase inhibitor treatment of said patient's cancer.

Further provided herein are methods for predicting therapeutic efficacy of TOR kinase inhibitor treatment of a patient having cancer, for example non-small cell lung carcinoma or cervical cancer, with a TOR kinase inhibitor, the method comprising: a) obtaining a biological test sample from the patient's cancer; b) obtaining the gene expression level(s) of one or more genes selected from Table 1 in said biological test sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of therapeutic efficacy of said TOR kinase inhibitor treatment for said patient.

Further provided herein are methods screening a patient having cancer, for example non-small cell lung carcinoma or cervical cancer, for LKB1 gene and/or protein loss and/or mutation, the method comprising: a) obtaining a biological test sample from the patient's cancer; b) obtaining the gene expression level(s) of one or more genes selected from Table 1 in said biological test sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of LKB1 gene and/or protein loss and/or mutation.

Further provided herein are methods for treating a tumor syndrome, for example Peutz-Jeghers Syndrome, comprising comparing a patient's gene expression level(s) to wild type, and administering an effective amount of a TOR kinase inhibitor to the patient having Peutz-Jeghers Syndrome characterized by a gene expression level characterized by higher similarity to the gene expression level of a reference sample with LKB1 gene and/or protein loss and/or mutation, than the gene expression level of a wild type sample without LKB1 gene and/or protein loss and/or mutation, and wherein the genes are selected from Table 1.

Further provided are methods for treating a tumor syndrome, for example Peutz-Jeghers Syndrome, comprising screening a patient for the presence of LKB1 gene and/or protein loss and/or mutation, relative to wild type, and administering an effective amount of a TOR kinase inhibitor to the patient having a tumor syndrome characterized by a gene expression level characterized by higher similarity to the gene expression level of a reference sample with LKB1 gene and/or protein loss and/or mutation, than the gene expression level of a wild type sample without LKB1 gene and/or protein loss and/or mutation, and wherein the genes are selected from Table 1.

Further provided herein are methods for predicting LKB1 gene and/or protein loss and/or mutation in a patient having a tumor syndrome, for example, Peutz-Jeghers Syndrome, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of an LKB1 gene and/or protein loss and/or mutation in the patient.

Further provided herein are methods for predicting response to TOR kinase inhibitor therapy in a patient having a tumor syndrome, for example, Peutz-Jeghers Syndrome, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of response to TOR kinase inhibitor treatment of said patient's tumor syndrome.

Further provided herein are methods for predicting therapeutic efficacy of treatment of a patient having a tumor syndrome, for example, Peutz-Jeghers Syndrome, with a TOR kinase inhibitor, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood of therapeutic efficacy of said TOR kinase inhibitor treatment for said patient.

Further provided herein are methods screening a patient having a tumor syndrome, for example Peutz-Jeghers Syndrome, for LKB1 gene and/or protein loss and/or mutation, comprising: a) obtaining a biological test sample from the patient; b) obtaining the gene expression level(s) of one or more genes selected from Table 1 in said biological test sample; c) comparing said gene expression level(s) to a set of reference levels that represent the gene expression level of a biological wild-type sample without LKB1 gene and/or protein loss and/or mutation, and the gene expression level of a reference sample with LKB1 gene and/or protein loss and/or mutation; wherein the gene expression level(s) of the biological test sample characterized by higher similarity to the gene expression level of a reference sample with LKB1 gene and/or protein loss and/or mutation, indicates an increased likelihood for LKB1 gene and/or protein loss and/or mutation.

In certain embodiments provided herein, the gene expression level of the biological test sample is obtained using gene mRNA measurement. In certain of the methods and embodiments provided herein, the gene expression level of the biological test sample is obtained using RT-PCR or Affymetrix HGU133plus2. In some embodiments, comparison of gene expression levels is performed using Prediction Analysis of Microarrays for R (“PAMR”) (http://cran.r-project.org/web/packages/pamr/pamr.pdf). In some embodiments, similarity between gene expression level(s) of a biological test sample with wild-type samples and/or reference samples is determined using PAMR.

Further provided herein are kits comprising one or more containers filled with a TOR kinase inhibitor or a pharmaceutical composition thereof, reagents for measuring gene expression levels of a patient's cancer or of a patient having a tumor syndrome and instructions for measuring gene expression levels of a patient's cancer or of a patient having a tumor syndrome. In one embodiment, the measurement comprises measurement of the expression level(s) of one or more genes from Table 1. In one embodiment, the gene expression measurement instructions are RT-PCT or Affymetrix HGU133plus2 instructions. In one embodiment, the kit further comprises instructions for comparing the expression levels to a set of reference levels that represent the gene expression levels of a biological wild-type sample without LKB1 gene and/or protein loss and/or mutation, and the gene expression level of a reference sample with LKB1 gene and/or protein loss and/or mutation. In one embodiment, the instructions for the comparison of expression levels are instructions for using PAMR.

In one embodiment, the LKB1 gene mutation or loss results in a decrease in LKB1 mRNA expression (e.g., relative to wild type). In another embodiment, the LKB1 gene mutation or loss results in a change in LKB1 mRNA structure (e.g., relative to wild type). In another embodiment, the LKB1 gene mutation or loss results in a decrease in LKB1 protein production (e.g., relative to wild type). In another embodiment, the LKB1 gene mutation or loss results in a change in LKB1 protein structure (e.g., relative to wild type). Types of gene mutations contemplated include mutations of the LKB1 DNA sequence in which the number of bases is altered, categorized as insertion or deletion mutations (frameshift mutations), and mutations of the DNA that change one base into another, categorized as missense mutations, which are subdivided into the classes of transitions (one purine to another purine, or one pyrimidine to another pyrimidine) and transversions (a purine to a pyrimidine, or a pyrimidine to a purine) and nonsense mutations, wherein a codon encoding an amino acid is changed to a stop codon, thus resulting in truncated protein.

In certain embodiments, the gene expression level(s), for example, in a biological test sample, as referenced herein is comprised of the expression level(s) of one or more of the genes set forth in Table 1. In a further embodiment, the gene expression level(s) does not include the expression level of IGF1R.

In certain embodiments, the gene expression levels associated with LKB1 gene and/or protein mutation and/or loss, for example in a biological test sample, are characterized by an upregulation of one or more genes indicated in Table 1 as having a negative Fold Change value and/or a downregulation of one or more genes in Table 1 as having a positive Fold Change value.

In a particular embodiment, the gene expression levels associated with LKB1 gene and/or protein mutation and/or loss, for example, in a biological test sample, is characterized by upregulation of one or more of the following genes: scavenger receptor class A, member 5 (putative); fibrinogen gamma chain; fibrinogen alpha chain; insulin-like 4 (placenta); organic solute transporter beta; phosphodiesterase 1A, calmodulin-dependent; carbamoyl-phosphate synthetase 1, mitochondrial; frizzled homolog 10 (Drosophila); mucin SAC, oligomeric mucus/gel-forming; trefoil factor 1; transient receptor potential cation channel, subfamily C, member 6; interleukin 1 receptor, type II; fibrinogen beta chain; chromosome 12 open reading frame 39; hypothetical gene supported by AK090616; R-spondin 3 homolog (Xenopus laevis); and interleukin 1 receptor, type II.

In a particular embodiment, the gene expression levels associated with LKB1 gene and/or protein mutation and/or loss, for example, in a biological test sample, are characterized by downregulation of one or more of the following genes: chitinase 3-like 1(cartilage glycoprotein-39); odz, odd Oz/ten-m homolog 2 (Drosophila); chemokine (C—C motif) ligand 5; bone morphogenetic protein 4; calcyphosine; Uncharacterized protein LOC100131897; and CD74 molecule, major histocompatibility complex, class II invariant chain.

In a particular embodiment, the gene expression levels associated with LKB1 gene and/or protein mutation and/or loss, for example in a biological test sample, are characterized by upregulation of one or more of the following genes: scavenger receptor class A, member 5 (putative); fibrinogen gamma chain; fibrinogen alpha chain; insulin-like 4 (placenta); organic solute transporter beta; phosphodiesterase 1A, calmodulin-dependent; carbamoyl-phosphate synthetase 1, mitochondrial; frizzled homolog 10 (Drosophila); mucin SAC, oligomeric mucus/gel-forming; trefoil factor 1; transient receptor potential cation channel, subfamily C, member 6; interleukin 1 receptor, type II; fibrinogen beta chain; chromosome 12 open reading frame 39; hypothetical gene supported by AK090616; R-spondin 3 homolog (Xenopus laevis); and interleukin 1 receptor, type II, and further characterized by downregulation of one or more of the following genes: chitinase 3-like 1 (cartilage glycoprotein-39); odz, odd Oz/ten-m homolog 2 (Drosophila); chemokine (C—C motif) ligand 5; bone morphogenetic protein 4; calcyphosine; Uncharacterized protein LOC 100131897; and CD74 molecule, major histocompatibility complex, class II invariant chain.

In one embodiment, the gene expression levels associated with LKB1 gene and/or protein mutation and/or loss, for example in a biological test sample, are characterized by upregulation of one or more of the following genes: homogentisate 1,2-dioxygenase (homogentisate oxidase); ATP-binding cassette, sub-family C(CFTR/MRP), member 2; chromosome 12 open reading frame 39; fibrinogen beta chain; fibrinogen gamma chain; R-spondin 3 homolog (Xenopus laevis); kynureninase (L-kynurenine hydrolase); carbamoyl-phosphate synthetase 1, mitochondrial; SPARC related modular calcium binding 1; interleukin 1 receptor, type II; chromosome 6 open reading frame 176; neuronal PAS domain protein 2; chondroitin sulfate N-acetylgalactosaminyltransferase 1; insulin-like 4 (placenta); nitric oxide synthase trafficker; and phosphodiesterase 4D, cAMP-specific (phosphodiesterase E3 dunce homolog, Drosophila). In some embodiments, the gene expression levels associated with LKB1 gene and/or protein mutation and/or loss, for example in a biological test sample, are characterized by downregulation of one or more of the following genes: bone morphogenetic protein 4; and pentraxin-related gene, rapidly induced by IL-1 beta.

In certain embodiments, gene expression is upregulated by a factor of about 2, about 5, about 10, about 20, about 30, about 40, about 50, about 60, about 70, about 80, about 90, about 100, about 110, about 120 or more relative to wild type. In certain embodiments, gene expression is downregulated by a factor of about 2, about 5, about 10, about 20, about 30, about 40, about 50, about 60, about 70, about 80, about 90, about 100, about 110, about 120 or more relative to wild type.

In certain embodiments, the cancer, for example non-small cell lung carcinoma or cervical cancer, or the tumor syndrome, for example Peutz-Jeghers Syndrome, results directly or indirectly from LKB1 gene and/or protein loss and/or mutation, relative to that of wild type.

In one embodiment, the LKB1 gene mutation is a somatic mutation.

In one embodiment, a patient or a patient's cancer is screened for LKB1 gene and/or protein loss and/or mutation by obtaining a biological sample from said patient or said patient's cancer, and measuring the gene expression level(s) of said sample ex vivo. In certain embodiments, the ex vivo analysis is performed using microarray analysis or sequence based techniques, such as serial analysis of gene expression (SAGE or SuperSAGE).

In certain of the methods and embodiments provided herein, the gene expression levels are measured using RT-PCR or Affymetrix HGU133plus2. In some embodiments, the gene expression levels are compared to wild type gene expression levels using the statistical package Prediction Analysis of Microarrays for R (“PAMR”). In some embodiments, similarity between gene expression level(s) of a biological test sample with wild-type samples and/or reference samples is determined using PAMR. In certain embodiments, the gene expression level is comprised of the gene expression levels of one or more of the genes set forth in Table 1.

In certain of the methods and embodiments provided herein, the gene expression level(s) (such as those of Table 1) is correlated with increased likelihood of LKB1 gene and/or protein loss and/or mutation.

A TOR kinase inhibitor can be combined with other pharmacologically active compounds (“second active agents”) in methods and compositions described herein. It is believed that certain combinations may work in the treatment of particular types of diseases or disorders, and conditions and symptoms associated with such diseases or disorders. A TOR kinase inhibitor can also work to alleviate adverse effects associated with certain second active agents, and vice versa.

One or more second active ingredients or agents can be used in the methods and compositions described herein. Second active agents can be large molecules (e.g., proteins) or small molecules (e.g., synthetic inorganic, organometallic, or organic molecules).

Examples of second active agents include, but are not limited to, agents that modulate AMP levels (e.g., an AMP activator), glucose uptake, metabolism or a stress response. In one embodiment, the second active agent is 2-deoxyglucose. In one embodiment, the second active agent is metformin. In one embodiment, the second active agent is phenformin. In another embodiment, the second active agent is pemetrexed (e.g., ALIMTA®).

Administration of a TOR kinase inhibitor and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for a TOR kinase inhibitor is oral. Preferred routes of administration for the second active agents or ingredients of the invention are known to those of ordinary skill in the art. See, e.g., Physicians' Desk Reference, 1755-1760 (56th ed., 2002).

In one embodiment, a second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount(s) of a TOR kinase inhibitor and any optional additional active agents concurrently administered to the patient.

Further provided herein are methods of reducing, treating and/or preventing adverse or undesired effects associated with conventional therapy including, but not limited to, surgery, chemotherapy, radiation therapy, hormonal therapy, biological therapy and immunotherapy. TOR kinase inhibitors and other active ingredients can be administered to a patient prior to, during, or after the occurrence of the adverse effect associated with conventional therapy.

### 5.6 Pharmaceutical Compositions and Routes of Administration

Provided herein are compositions comprising an effective amount of a TOR kinase inhibitor and compositions comprising an effective amount of a TOR kinase inhibitor and a pharmaceutically acceptable carrier or vehicle. In some embodiments, the pharmaceutical composition described herein are suitable for oral, parenteral, mucosal, transdermal or topical administration.

The TOR kinase inhibitors can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the TOR kinase inhibitor in the pharmaceutical composition may be at a level that will exercise the desired effect; for example, about 0.005 mg/kg of a patient's body weight to about 10 mg/kg of a patient's body weight in unit dosage for both oral and parenteral administration.

The dose of a TOR kinase inhibitor to be administered to a patient is rather widely variable and can be patient to the judgment of a health-care practitioner. In general, the TOR kinase inhibitors can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight or about 0.25 mg/kg of a patient's body weight to about 0.5 mg/kg of a patient's body weight. In one embodiment, one dose is given per day. In any given case, the amount of the TOR kinase inhibitor administered will depend on such factors as the solubility of the active component, the formulation used and the route of administration.

In another embodiment, provided herein are methods for the treatment or prevention of a disease or disorder comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a TOR kinase inhibitor to a patient in need thereof.

In another embodiment, provided herein are methods for the treatment or prevention of a disease or disorder comprising the administration of about 1 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of a TOR kinase inhibitor to a patient in need thereof. In a particular embodiment, the methods disclosed herein comprise the administration of 400 mg/day, 600 mg/day or 800 mg/day of a TOR kinase inhibitor to a patient in need thereof.

In another embodiment, provided herein are unit dosage formulations that comprise between about 1 mg and about 2000 mg, about 1 mg and 200 mg, about 35 mg and about 1400 mg, about 125 mg and about 1000 mg, about 250 mg and about 1000 mg, or about 500 mg and about 1000 mg of a TOR kinase inhibitor.

In a particular embodiment, provided herein are unit dosage formulation comprising about 100 mg or 400 mg of a TOR kinase inhibitor.

In another embodiment, provided herein are unit dosage formulations that comprise 1 mg, 2.5 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a TOR kinase inhibitor.

A TOR kinase inhibitor can be administered once, twice, three, four or more times daily.

A TOR kinase inhibitor can be administered orally for reasons of convenience. In one embodiment, when administered orally, a TOR kinase inhibitor is administered with a meal and water. In another embodiment, the TOR kinase inhibitor is dispersed in water or juice (e.g., apple juice or orange juice) and administered orally as a suspension. In another embodiment, when administered orally, a TOR kinase inhibitor is administered in a fasted state.

The TOR kinase inhibitor can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a TOR kinase inhibitor without an additional carrier, excipient or vehicle.

In another embodiment, provided herein are compositions comprising an effective amount of a TOR kinase inhibitor and a pharmaceutically acceptable carrier or vehicle, wherein a pharmaceutically acceptable carrier or vehicle can comprise an excipient, diluent, or a mixture thereof. In a further embodiment, provided herein are compositions comprising an effective amount of a TOR kinase inhibitor, and a pharmaceutically acceptable carrier or vehicle, and one or more agents that modulate AMP levels, glucose uptake, metabolism or a stress response. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a TOR kinase inhibitor with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a TOR kinase inhibitor as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the TOR kinase inhibitor can be delayed or prolonged by proper formulation. For example, a slowly soluble pellet of the TOR kinase inhibitor can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the TOR kinase inhibitor in oily or emulsified vehicles that allow it to disperse slowly in the serum.

### 6. EXAMPLES

### 6.1 Gene Expression

Gene Expression Analysis.

40 NSCLC cell lines were grouped into two groups, namely LKB1 positive and LKB1 negative cell lines based on quantified western measurements, wherein cell lines with LKB1/Act protein ratio larger than 25 were classified as LKB1 positive, and cell lines with LKB1/Act protein ratio less than 25 were classified as LKB1 negative.

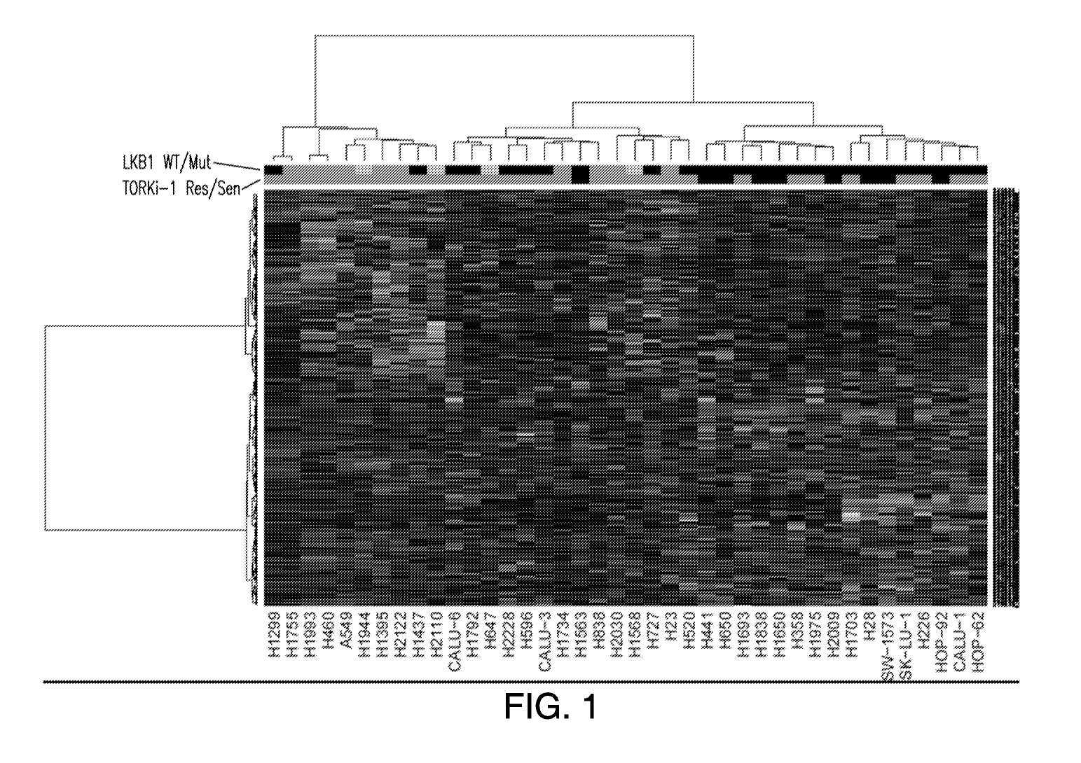

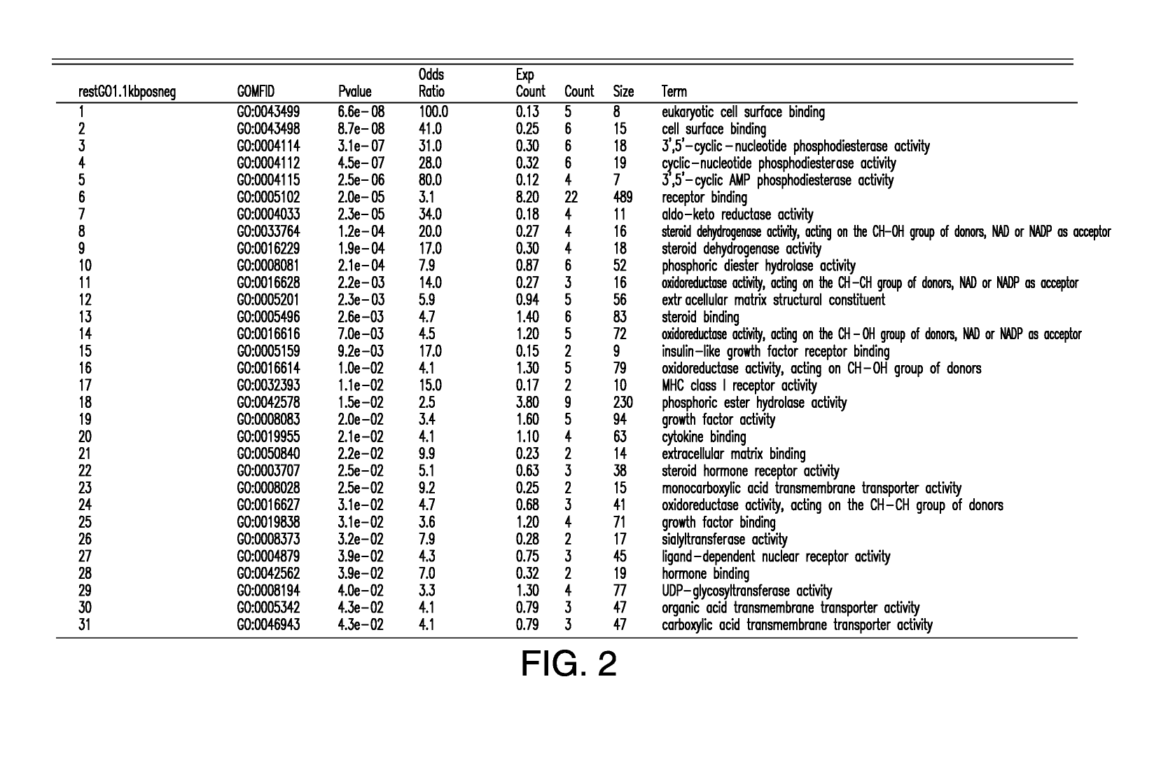

The free software R package PAMR was used, which implements “nearest shrunken centroids” (see: PNAS 99 (10): 6567-6572 (2002)) to identify subsets of genes that distinguish LKB1 positive from LKB1 negative NSCLC cell lines. PAMR selected 463 probes with 10-fold cross validation error at 22% (78% accuracy). After removing probes that had a small fold difference between the two groups (<1.5 fold), a 458-probe signature was obtained. Results are set forth in Table 1 and FIGS. 1-2.

This experiment demonstrates that a particular gene expression level is associated with the loss of LKB1.

A number of references have been cited, the disclosures of which are incorporated herein by reference in their entirety.

What is claimed is:

1. A method for predicting LKB1 gene or protein loss or mutation in a patient's cancer, comprising:
a) obtaining a biological test sample from the patient's cancer;
b) obtaining the gene expression level of one or more genes selected from Table 1 in said biological sample;
c) comparing said gene expression level to a set of reference levels that represent the gene expression level of a biological wild-type sample without LKB1 gene or protein loss or mutation, and the gene expression level of a reference sample with LKB1 gene or protein loss or mutation;
wherein the gene expression level of the biological test sample characterized by higher similarity to the gene expression level of a reference sample with LKB1 gene or protein loss or mutation, indicates an increased likelihood of an LKB1 gene or protein loss or mutation in the patient's cancer.

2. The method of claim 1, wherein the gene expression level of the biological test sample is obtained using gene mRNA measurement.

3. The method of claim 1, wherein the gene expression level of the biological test sample is obtained using RT-PCR or Affymetrix HGU133plus2.

4. The method of claim 1, wherein the comparison of gene expression levels is performed using PAMR.

5. The method of claim 1, wherein the cancer is non-small cell lung carcinoma or cervical cancer.

6. The method of claim 1, wherein the gene expression level of the biological test sample is characterized by an upregulation of one or more genes in Table 1 as having a negative Fold Change value.

7. The method of claim 1, wherein the gene expression level of the biological test sample is characterized by a downregulation of one or more genes in Table 1 as having a positive Fold Change value.

8. The method of claim 1, wherein the gene expression level of the biological test sample is characterized by upregulation of one or more of the following genes: scavenger receptor class A, member 5 (putative); fibrinogen gamma chain; fibrinogen alpha chain; insulin-like 4 (placenta); organic solute transporter beta; phosphodiesterase 1A, calmodulin-dependent; carbamoyl-phosphate synthetase 1, mitochondrial; frizzled homolog 10 (Drosophila); mucin SAC, oligomeric mucus/gel-forming; trefoil factor 1; transient receptor potential cation channel, subfamily C, member 6; interleukin 1 receptor, type II; fibrinogen beta chain; chromosome 12 open reading frame 39; hypothetical gene supported by AK090616; R-spondin 3 homolog (Xenopus laevis); and interleukin 1 receptor, type II.

9. The method of claim 1, wherein the gene expression level is characterized by downregulation of one or more of the following genes: chitinase 3-like 1 (cartilage glycoprotein-39); odz, odd Oz/ten-m homolog 2 (Drosophila); chemokine (C—C motif) ligand 5; bone morphogenetic protein 4; calcyphosine; Uncharacterized protein LOC100131897; and CD74 molecule, major histocompatibility complex, class II invariant chain.

10. A method for treating non-small cell lung carcinoma, cervical cancer or Peutz-Jeghers Syndrome, comprising administering an effective amount of a TOR kinase inhibitor to a patient having non-small cell lung carcinoma, cervical cancer or Peutz-Jeghers Syndrome, wherein the gene expression level of a biological test sample from said patient is characterized by higher similarity to the gene expression level of a reference sample with LKB1 gene or protein loss or mutation than the gene expression level of a wild type sample without LKB1 gene or protein loss or mutation, and wherein the genes are selected from Table 1.

11. The method of claim 10, wherein the gene expression level of the biological test sample is obtained using gene mRNA measurement.

12. The method of claim 10, wherein the gene expression level of the biological test sample is obtained using RT-PCR or Affymetrix HGU133plus2.

13. The method of claim 10, wherein the comparison of expression levels is performed using PAMR.

14. The method of claim 10, wherein the gene expression level of the biological test sample is characterized by an upregulation of one or more genes indicated in Table 1 as having a negative Fold Change value.

15. The method of claim 10, wherein the gene expression level of the biological test sample is characterized by a downregulation of one or more genes in Table 1 as having a positive Fold Change value.

16. The method of claim 10, wherein the gene expression level of the biological test sample is characterized by upregulation of one or more of the following genes: scavenger receptor class A, member 5 (putative); fibrinogen gamma chain; fibrinogen alpha chain; insulin-like 4 (placenta); organic solute transporter beta; phosphodiesterase 1A, calmodulin-dependent; carbamoyl-phosphate synthetase 1, mitochondrial; frizzled homolog 10 (Drosophila); mucin SAC, oligomeric mucus/gel-forming; trefoil factor 1; transient receptor potential cation channel, subfamily C, member 6; interleukin 1 receptor, type II; fibrinogen beta chain; chromosome 12 open reading frame 39; hypothetical gene supported by AK090616; R-spondin 3 homolog (Xenopus laevis); and interleukin 1 receptor, type II.

17. The method of claim 10, wherein the gene expression level of the biological test sample is characterized by downregulation of one or more of the following genes: chitinase 3-like 1 (cartilage glycoprotein-39); odz, odd Oz/ten-m homolog 2 (Drosophila); chemokine (C—C motif) ligand 5; bone morphogenetic protein 4; calcyphosine; Uncharacterized protein LOC100131897; and CD74 molecule, major histocompatibility complex, class II invariant chain.

18. A method for treating non-small cell lung carcinoma or cervical cancer, comprising screening a patient's carcinoma or cancer for the presence of LKB1 gene or protein loss or mutation, relative to wild type, and administering an effective amount of a TOR kinase inhibitor to the patient having non-small cell lung carcinoma or cervical cancer characterized by a gene expression level characterized by higher similarity to the gene expression level of a reference sample with LKB1 gene or protein loss or mutation than the gene expression level of a wild type sample without LKB1 gene or protein loss or mutation, and wherein the genes are selected from Table 1.

19. The method of claim 18, wherein the gene expression level is obtained using gene mRNA measurement.

20. The method of claim 18, wherein the gene expression level is obtained using RT-PCR or Affymetrix HGU133plus2.

21. The method of claim 18, wherein the comparison of expression levels is performed using PAMR.

22. The method of claim 18, wherein the gene expression level of the patient's carcinoma or cancer is characterized by upregulation of one or more of the following genes: scavenger receptor class A, member 5 (putative); fibrinogen gamma chain; fibrinogen alpha chain; insulin-like 4 (placenta); organic solute transporter beta; phosphodiesterase 1A, calmodulin-dependent; carbamoyl-phosphate synthetase 1, mitochondrial; frizzled homolog 10(Drosophila); mucin SAC, oligomeric mucus/gel-forming; trefoil factor 1; transient receptor potential cation channel, subfamily C, member 6; interleukin 1 receptor, type II; fibrinogen beta chain; chromosome 12 open reading frame 39; hypothetical gene supported by AK090616; R-spondin 3 homolog (Xenopus laevis); and interleukin 1 receptor, type II.

23. The method of claim 18, wherein the gene expression level of the patient's carcinoma or cancer is characterized by downregulation of one or more of the following genes: chitinase 3-like 1 (cartilage glycoprotein-39); odz, odd Oz/ten-m homolog 2 (Drosophila); chemokine (C—C motif) ligand 5; bone morphogenetic protein 4; calcyphosine; Uncharacterized protein LOC100131897; and CD74 molecule, major histocompatibility complex, class II invariant chain.

24. A method for treating Peutz-Jeghers Syndrome, comprising comparing a patient's gene expression level to wild type, and administering an effective amount of a TOR kinase inhibitor to the patient having Peutz-Jeghers Syndrome characterized by a gene expression level characterized by higher similarity to the gene expression level of a reference sample with LKB1 gene or protein loss or mutation than the gene expression level of a wild type sample without LKB1 gene or protein loss or mutation, and wherein the genes are selected from Table 1.

25. The method of claim 24, wherein the gene expression level is obtained using gene mRNA measurement.

26. The method of claim 24, wherein the gene expression level is obtained using RT-PCR or Affymetrix HGU133plus2.

27. The method of claim 24, wherein the comparison of gene expression levels is performed using PAMR.

28. The method of claim 24, wherein the patient's gene expression level is characterized by upregulation of one or more of the following genes: scavenger receptor class A, member 5 (putative); fibrinogen gamma chain; fibrinogen alpha chain; insulin-like 4 (placenta); organic solute transporter beta; phosphodiesterase 1A, calmodulin-dependent; carbamoyl-phosphate synthetase 1, mitochondrial; frizzled homolog 10 (Drosophila); mucin SAC, oligomeric mucus/gel-forming; trefoil factor 1; transient receptor potential cation channel, subfamily C, member 6; interleukin 1 receptor, type II; fibrinogen beta chain; chromosome 12 open reading frame 39; hypothetical gene supported by AK090616; R-spondin 3 homolog (Xenopus laevis); and interleukin 1 receptor, type II.

29. The method of claim 24, wherein the patient's gene expression level is characterized by downregulation of one or more of the following genes: chitinase 3-like 1(cartilage glycoprotein-39); odz, odd Oz/ten-m homolog 2 (Drosophila); chemokine (C—C motif) ligand 5; bone morphogenetic protein 4; calcyphosine; Uncharacterized protein LOC 100131897; and CD74 molecule, major histocompatibility complex, class II invariant chain.

30. A method of predicting response to treatment with a TOR kinase inhibitor in a patient having cancer, the method comprising:
a) obtaining a biological test sample from the patient's cancer;
b) obtaining the gene expression level of one or more genes selected from Table 1 in said biological test sample;
c) comparing said gene expression level to a set of reference levels that represent the gene expression level of a biological wild-type sample without LKB1 gene or protein loss or mutation and the gene expression level of a reference sample with LKB1 gene or protein loss or mutation;
wherein the gene expression level of the biological test sample characterized by higher similarity to the gene expression level of a reference sample with LKB1 gene or protein loss or mutation, indicates an increased likelihood of response to TOR kinase inhibitor treatment of said patient's cancer.

31. The method of claim 30, wherein the gene expression level of the biological test sample is obtained using gene mRNA measurement.

32. The method of claim 30, wherein the gene expression level of the biological test sample is obtained using RT-PCR or Affymetrix HGU133plus2.

33. The method of claim 30, wherein the comparison of gene expression levels is performed using PAMR.

34. A kit comprising one or more containers filled with a TOR kinase inhibitor or a pharmaceutical composition thereof, reagents for measuring gene expression levels of a patient's cancer or of a patient having a tumor syndrome and instructions for measuring gene expression levels of a patient's cancer or of a patient having a tumor syndrome.

35. The kit of claim 34, wherein the measurement comprises measurement of the expression level one or more genes from Table 1.

36. The kit of claim 34, wherein the gene expression measurement instructions are RT-PCT or Affymetrix HGU133plus2 instructions.

37. The kit of claim 35, further comprising instructions for comparing the expression levels to a set of reference levels that represent the gene expression levels of a biological wild-type sample without LKB1 gene or protein loss or mutation and the gene expression level of a reference sample with LKB1 gene or protein loss or mutation.

38. The kit of claim 37, wherein the instructions for the comparison of expression levels are instructions for using PAMR.
